(19) United States
(12) Patent Application Publication
(10) Pub. No.: US 2024/0279316 A1
(43) Pub. Date: Aug. 22, 2024

(54) COMPOSITIONS AND METHODS FOR TREATING ALPHA-SYNUCLEINOPATHIES

(71) Applicants: ASTRAZENECA AB, Södertälje (SE); TAKEDA PHARMACEUTICAL COMPANY LIMITED, Osaka (JP)

(72) Inventors: Wei YIN, Belmont, MA (US); Arthur SIMEN, Dover, MA (US); Antonio LAURENZA, Union Mills, NC (US); Stephen ZICHA, Belmont, MA (US); Jaya PADMANABHAN, Cambridge, MA (US); Elena RATTI, Cambridge, MA (US); Michael PERKINTON, Cambridge (GB); Ian GURRELL, Cambridge (GB); Tris VAUGHAN, Cambridge (GB); Keith TAN, Cambridge (GB); Thor OSTENFELD, Cambridge (GB)

(21) Appl. No.: 18/398,531

(22) Filed: Dec. 28, 2023

Related U.S. Application Data
(60) Provisional application No. 63/477,709, filed on Dec. 29, 2022.

(51) Int. Cl. C07K 16/18

(52) U.S. Cl. CPC C07K 16/18; C07K 2317/52; C07K 2317/565; C07K 2317/90

(57) ABSTRACT

The invention provides a method for treating or preventing an a-synucleinopathy in a subject in need thereof, the method comprising administering to the subject a fixed dose of 50-5,000 mg of an anti-α-synuclein antibody, or antigen-binding fragment thereof. Also provided are corresponding compositions and kits.

### BACKGROUND

The present invention relates to α-synuclein antibodies and their use in the prevention or treatment of disease, in particular alpha-synucleinopathies, and more particularly Parkinson's disease (PD) and Multiple System Atrophy (MSA).

Alpha-synucleinopathies (or α-synucleinopathies), also known as Lewy body diseases (LBDs), are a family of neurodegenerative diseases that all have at their core alpha-synuclein as the key pathological hallmark (Jellinger (2003) Mov. Disord. 18 Suppl 6: S2-12; and Spillantini and Goedert (2000) Ann. N. Y. Acad. Sci. 920: 16-27; both of which are incorporated herein by reference). Alpha-synucleinopathies include Parkinson's disease (PD), dementia with Lewy bodies (DLB) and multiple system atrophy (MSA).

PD is a slowly progressive age-related movement disorder affecting greater than 1% of people over 65 years old. The hallmark motor symptoms of PD include resting tremor, postural instability, bradykinesia, and rigidity. However, PD also involves nonmotor symptoms including anosmia, cognitive dysfunction, sleep-awake dysregulation, dysautonomia, depression and constipation. PD is defined by the selective loss of dopaminergic neurons along with the widespread accumulation of α-synuclein-containing aggregates in surviving neurons, termed Lewy bodies. PD is the second most common neurodegenerative condition after Alzheimer's disease.

MSA is a rare but universally fatal progressive neurodegenerative disorder clinically defined by parkinsonian, cerebellar, and dysautonomic features. Isolated autonomic dysfunction with predominant genitourinary dysfunction and orthostatic hypotension and REM sleep behavior disorder are common characteristics of the initial stages of MSA. Like PD, MSA is characterized by widespread accumulation of α-synuclein aggregates. Disease-modifying therapies to halt or slow the progression of MSA are currently unavailable.

A defining hallmark pathology of alpha-synucleinopathies are Lewy bodies and Lewy neurites, which are insoluble inclusions of aggregated proteins found inside neurons of the brain revealed upon post-mortem histopathological examination. The presence of Lewy pathology and neuronal loss in non-motor brain regions such as the basal forebrain, mesopontine system, amygdala, neocortex, dorsal motor nucleus of the vagus nerve, olfactory bulbs, locus coeruleus, and the brainstem, may cause cognitive deficits and dementia, hyposmia, sleep disturbances including rapid eye movement sleep behaviour disorder (RBD), mood disorders including depression and anxiety, autonomic dysfunction including cardiovascular and gastrointestinal problems such as constipation, and fatigue and somnolence. Some of these non-motor symptoms appear to characterise the premotor or prodromal phase of Parkinson's disease (Kalia et al. Lancet (2015), 386(9996): 896-912; incorporated herein by reference).

The presence of Lewy pathology and neuronal loss in motor brain regions, including most notably the death of dopaminergic neurons in the substantia nigra, may cause resting tremor, rigidity, bradykinesia and postural instability, the hallmarks of Parkinson Disease (Spillantini and Goedert, Ann N Y Acad Sci (2000), 920: 16-27; incorporated herein by reference).

Alpha-synuclein (also called “α-synuclein” or “α-syn”) protein is the major structural component of Lewy bodies and Lewy neurites. Alpha-synuclein is a small acidic protein made up of 140 amino acids (14 kDa). Human natural wild-type alpha-synuclein has the amino acid sequence SEQ ID NO: 1 as described under UniProtKB accession number P37840. Unless otherwise apparent from the context, reference to alpha-synuclein or its fragments includes the natural human wild-type amino acid sequence indicated above, and human allelic variants thereof, in particular those associated with Lewy body disease (e.g., E46K, A30P, H50Q, G51D and A53T, where the first letter indicates the amino acid in SEQ ID NO: 1, the number is the codon position in SEQ ID NO: 1, and the second letter is the amino acid in the allelic variant). Such variants can optionally be present individually or in any combination. The induced mutations E83Q, A90V. A76T, which enhance alpha-synuclein aggregation, can also be present individually or in combination with each other and/or with human allelic variants E46K. A30P, H50Q, G51D and A53T. At the structural level, alpha-synuclein contains three distinct regions: an amphipathic N-terminal alpha-helix domain that has lipid and membrane binding properties (residues 1-60), a central hydrophobic amyloid-binding domain that encodes the non-amyloid-beta component (NAC) of plaques (residues 61-95), and an acidic proline-rich C-terminal tail (residues 96-140). Residues 71-82 of the NAC domain are believed to be key to the aggregation/fibrillation properties of alpha-synuclein by enabling the protein to switch from a random coil structure to a beta-sheet structure (Bisaglia et al. (2009) FASEB J. 23(2): 329-40; incorporated herein by reference). Although the C-terminal domain is free of significant secondary structure it contains a key phosphorylation site at residue Ser129 and a number of tyrosine residues that are nitrated in cytosolic alpha-synuclein inclusions. N-terminal and C-terminal truncated forms of alpha-synuclein also exist. Post-translational modifications to the protein can affect alpha-synuclein aggregation and toxicity (Oueslati et al. (2010) Prog. Brain. Res. 183: 115-45; incorporated herein by reference).

Under pathological conditions, aberrant alpha-synuclein aggregation may be key to the pathological changes seen in alpha-synucleinopathies (Lashuel et al. (2002) Nature 418: 291; and Tsigelny et al. (2007) FEBS J. 274: 1862-1877; both of which are incorporated herein by reference). In-vitro studies have shown that alpha-synuclein monomers may form the starting point for the aggregation process. The monomer can aggregate into a variety of small oligomeric species that are then stabilised by beta-sheet interactions, going on to form protofibrils which can polymerise into insoluble fibrillary structures reminiscent of those identified in Lewy bodies (Cremades et al. (2012) Cell (2012), 149(5): 1048-59; incorporated herein by reference). In vitro and in vivo studies have shown that the neurotoxic effects of alpha-synuclein appear to be elicited by small soluble oligomeric conformers or protofibrils (Winner et al. (2011) Proc. Natl. Acad. Sci. USA 108(10): 4194-9; and Danzer et al. (2007) J. Neurosci. 27(34): 9220-32; both of which are incorporated herein by reference). While fibrillar aggregates of alpha-synuclein are characteristic of PD, oligomeric forms of alpha-synuclein are the toxic species (Danzer et al. (2007) J. Neurosci. 27(34): 9220-32; Lashuel et al. (2002) Nature 418: 291; and Winner et al. (2011) Proc. Natl. Acad. Sci. USA 108: 4194-4199; each of which are incorporated herein by reference).

Alpha-synuclein oligomers can be released to the extracellular environment and taken up by neighboring cells in a “propagation” mechanism (Angot and Brundin, (2009) Parkinsonism Relat. Disord. 15 Suppl 3: S143-147; Desplats et al. (2009) Proc. Natl. Acad. Sci. USA 106: 13010-13015; and Lee et al. (2010) J. Biol. Chem. 285: 9262-9272; each of which are incorporated herein by reference). Aggregates of alpha-synuclein can propagate misfolding through a prion-like spreading mechanism (Lee et al. (2010) Nat. Rev. Neurol. 6: 702-706; Luk et al. (2012) J. Exp. Med. 209(5): 975-86; and Luk et al. (2012) Science 338(6109): 949-53; each of which are incorporated herein by reference). Alpha-synuclein can therefore induce neurodegeneration by either oligomer toxicity or propagation and prion-like spreading.

It is now well established and accepted that under conditions of cellular stress, and through this release of alpha-synuclein into the extracellular milieu, pathological transmissible forms of alpha-synuclein may be propagated between neurons (Recasens and Dehay, Front Neuroanat (2014), 8: 159; incorporated herein by reference).

Approaches to treat alpha-synucleinopathies have relied on passive immunotherapy. Passive immunotherapy approaches with antibodies targeting alpha-synuclein have been tested in numerous preclinical alpha-synucleinopathy mouse models (Lawand et al. (2015) Expert Opin. Ther. Targets 19: 1-10; incorporated herein by reference). Specifically, a study using a monoclonal antibody directed against alpha-synuclein (9E4) has shown in vivo clearance of alpha-synuclein aggregates and pathology, behavioural motor improvements, and neuroprotective effects (WO 2014/058924; which is incorporated herein by reference).

Further studies using passive immunisation of alpha-synuclein transgenic mice developed as experimental models of PD/DLB have shown the 9E4 monoclonal antibody to clear the alpha-synuclein pathology, decrease synaptic and axonal deficits, abrogate loss of striatal tyrosine hydroxylase fibres, and significantly reduce memory deficits and motor function impairments (Games et al. (2014) J. Neurosci. 34(28): 9441-54; Bae et al. (2012) J. Neurosci. 32(39): 13454-69; and Masliah et al. (2011) PLOS One 6(4): e19338; each of which are incorporated herein by reference). Further, passive administration of anti-alpha-synuclein monoclonal antibodies in wild-type mice that were injected intrastriatally with synthetic alpha-synuclein preformed fibrils (pffs) led to robust reduction in Lewy pathology, prevention of dopamine neuron loss in the substantia nigra, and a significant improvement in motor impairments that are manifest in the mouse model after pffs treatment (Tran et al. (2014) Cell Rep. 7(6): 2054-65; incorporated herein by reference).

However, no passive immunotherapy approach has yet been approved for PD and other α-synucleinopathies. Current approaches to these diseases are therefore mainly focused on treating the motor-related symptoms of the diseases.

Accordingly, there is a need in the art for a therapy for treating alpha-synucleinopathies, particularly in humans.

### SUMMARY

The present invention relates to an anti-α-synuclein antibody, or an antigen-binding fragment thereof. The invention relates to the treatment or prevention of an α-synucleinopathy, using such an antibody or antibody fragment. In particular, the invention relates to particular dosage regimens that can be used when an anti-α-synuclein antibody, or an antigen-binding fragment thereof, is administered for treating or preventing an α-synucleinopathy. More particularly, the invention relates to the treatment of an α-synucleinopathy comprising administration of a fixed dose of an anti-α-synuclein antibody, or an antigen-binding fragment thereof, to a subject, e.g., a dose that is not dependent on the weight of the subject.

Accordingly, the invention provides a method for treating or preventing an α-synucleinopathy in a subject in need thereof, comprising administering to the subject a fixed dose of 50-5,000 mg of an anti-α-synuclein antibody, or antigen-binding fragment thereof. The invention also provides an anti-α-synuclein antibody, or antigen-binding fragment thereof, for use in a method of treating or preventing an α-synucleinopathy in a subject, the method comprising administering to the subject a fixed dose of 50-5,000 mg of the anti-α-synuclein antibody, or antigen-binding fragment thereof. The invention also provides use of an anti-α-synuclein antibody, or antigen-binding fragment thereof, in the manufacture of a medicament for treating or preventing an α-synucleinopathy in a subject, wherein the medicament comprises a fixed dose of 50-5,000 mg of said antibody or antigen-binding fragment thereof, or wherein said treating or preventing comprises administering a fixed dose of 50-5,000 mg of said antibody or antigen-binding fragment thereof. The invention also provides a composition comprising a fixed dose of 50-5,000 mg of an anti-α-synuclein antibody, or antigen-binding fragment thereof, for use in a method of treating or preventing an α-synucleinopathy. The invention also provides a kit comprising an anti-α-synuclein antibody, or antigen-binding fragment thereof, and instructions for use of the same for treating an α-synucleinopathy at a fixed dose of 50-5,000 mg.

The fixed dose of the anti-α-synuclein antibody, or antigen-binding fragment thereof, may be 70-4,500 mg. The fixed dose of the anti-α-synuclein antibody, or antigen-binding fragment thereof, may be 1,000-4,500 mg. The fixed dose of the anti-α-synuclein antibody, or antigen-binding fragment thereof, may be 1,800-2,600 mg. The fixed dose of the anti-α-synuclein antibody, or antigen-binding fragment thereof, may be 2,000-2,400 mg. The fixed dose of the anti-α-synuclein antibody, or antigen-binding fragment thereof, may be up to 2,600 mg. The fixed dose of the anti-α-synuclein antibody, or antigen-binding fragment thereof, may be up to 2,400 mg. The fixed dose of the anti-α-synuclein antibody, or antigen-binding fragment thereof, may be up to 2,200 mg. The fixed dose of the anti-α-synuclein antibody, or antigen-binding fragment thereof, may be up to 2,000 mg. The fixed dose of the anti-α-synuclein antibody, or antigen-binding fragment thereof, may be 1,400 mg. The fixed dose of the anti-α-synuclein antibody, or antigen-binding fragment thereof, may be 1,600 mg. The fixed dose of the anti-α-synuclein antibody, or antigen-binding fragment thereof, may be 1,700 mg. The fixed dose of the anti-α-synuclein antibody, or antigen-binding fragment thereof, may be 1,800 mg. The fixed dose of the anti-α-synuclein antibody, or antigen-binding fragment thereof, may be 2,000 mg or 2,400 mg. The fixed dose of the anti-α-synuclein antibody, or antigen-binding fragment thereof, may be 2,000 mg. The fixed dose of the anti-α-synuclein antibody, or antigen-binding fragment thereof, may be 2,400 mg. The fixed dose of the anti-α-synuclein antibody, or antigen-binding fragment thereof, may be 4,500 mg.

The method may comprise administering the anti-α-synuclein antibody, or antigen-binding fragment thereof, intravenously, subcutaneously, intradermally, or intramuscularly. Preferably, the method may comprise administering the anti-α-synuclein antibody, or antigen-binding fragment thereof, intravenously.

The anti-α-synuclein antibody, or antigen-binding fragment thereof, may be administered to the subject once every 3-5 weeks. The anti-α-synuclein antibody, or antigen-binding fragment thereof, may be administered to the subject multiple times over a period of at least 3 months, 6 months, 9 months, 1 year, 2 years or 5 years. The anti-α-synuclein antibody, or antigen-binding fragment thereof, may be administered to the subject once every four weeks over a period of at least 3 months, 6 months, 9 months, 1 year, 2 years or 5 years.

The α-synucleinopathy may be selected from Parkinson's disease (PD), dementia with Lewy bodies (DLB), multiple system atrophy (MSA), Alzheimer's Disease, pure autonomic failure, REM behavior disorder, a prodromal synucleinopathy and a neuroaxonal dystrophy. The α-synucleinopathy may be Parkinson's Disease (PD). The α-synucleinopathy may be multiple system atrophy (MSA). The α-synucleinopathy may be dementia with Lewy bodies (DLB). The subject may have a diagnosis of possible or probable MSA. The subject to be treated may have a Unified Multiple System Atrophy Rating Scale (UMSARS) Part I score of 21 or less. The subject to be treated may have a severity score of 2 or less on the swallowing item; a severity score of 2 or less on the ambulation item; and/or a severity score of 2 or less on the falling item as measured using a Unified Multiple System Atrophy Rating Scale (UMSARS) Part I score. The subject to be treated may have a UMSARS Part IV disability score of 3 or less.

The method may reduce progression of the α-synucleinopathy as measured by the subject's score in a modified version of the UMSARS Part I. The method may reduce progression of the α-synucleinopathy as measured by the subject's UMSARS total score. The method may reduce progression of the α-synucleinopathy as measured by the subject's UMSARS Part I score. The method may reduce progression of the α-synucleinopathy as measured by the subject's UMSARS Part II score. The method may reduce progression of the α-synucleinopathy as measured by the subject's 11-item UMSARS score. The method may reduce progression of the α-synucleinopathy as measured by the subject's Clinical Global Impression-Severity (CGI-S) scale score.

The method may reduce progression of the α-synucleinopathy as measured by α-synuclein spreading in the subject. The method may reduce progression of the α-synucleinopathy as measured by free unbound α-synuclein levels in the cerebrospinal fluid (CSF) of the subject.

The method may reduce the subject's score in a modified version of the Unified Multiple System Atrophy Rating Scale (UMSARS) Part I. The method may reduce the subject's Unified Multiple System Atrophy Rating Scale (UMSARS) Part I score. The method may reduce the subject's Unified Multiple System Atrophy Rating Scale (UMSARS) Part II score. The method may reduce the subject's 11-item Unified Multiple System Atrophy Rating Scale (UMSARS) score. The method may reduce the subject's Clinical Global Impression-Severity (CGI-S) scale score.

The method may reduce α-synuclein spreading in the subject. The method may reduce free unbound α-synuclein levels in the cerebrospinal fluid (CSF) of the subject.

The anti-α-synuclein antibody, or antigen-binding fragment thereof, may bind free unbound α-synuclein in the CSF of the subject. The anti-α-synuclein antibody, or antigen-binding fragment thereof, may bind monomeric and/or oligomeric α-synuclein in the CSF of the subject. The anti-α-synuclein antibody, or antigen-binding fragment thereof, may bind α-synuclein in the CSF of the subject in vivo.

The anti-α-synuclein antibody, or antigen-binding fragment thereof, may specifically bind the region comprised between about amino acid 102 and about amino acid 130 within the C-terminal region of human α-synuclein. The anti-α-synuclein antibody, or antigen-binding fragment thereof, may bind monomeric and aggregated forms of α-synuclein. The anti-α-synuclein antibody, or antigen-binding fragment thereof, may bind monomeric and aggregated forms of α-synuclein in vivo. The anti-α-synuclein antibody, or antigen-binding fragment thereof, may sequester both monomeric and aggregated forms of α-synuclein. The anti-α-synuclein antibody, or antigen-binding fragment thereof, may sequester both monomeric and aggregated forms of α-synuclein in vivo.

The anti-α-synuclein antibody, or antigen-binding fragment thereof, may bind monomeric human α-synuclein with a K_{D} of less than 500 pM.

The anti-α-synuclein antibody, or antigen-binding fragment thereof, may reduce α-synuclein spreading in vivo.

The anti-α-synuclein antibody, or antigen-binding fragment thereof, may comprise three heavy chain CDRs having sequences H-CDR1 of SEQ ID NO: 5; H-CDR2 of SEQ ID NO: 15; and H-CDR3 of SEQ ID NO: 16; and three light chain CDRs having sequences L-CDR1 of SEQ ID NO: 20; L-CDR2 of SEQ ID NO: 10; and L-CDR3 of SEQ ID NO: 21.

The anti-α-synuclein antibody, or antigen-binding fragment thereof, may comprise a variable heavy chain region comprising an amino acid sequence that is at least 90% identical to the amino acid sequence of SEQ ID NO: 14. The anti-α-synuclein antibody, or antigen-binding fragment thereof, may comprise a variable heavy chain region comprising the amino acid sequence of SEQ ID NO: 14. The anti-α-synuclein antibody, or antigen-binding fragment thereof, may comprise a variable light chain region comprising an amino acid sequence that is at least 90% identical to the amino acid sequence of SEQ ID NO: 19. The anti-α-synuclein antibody, or antigen-binding fragment thereof, may comprise a variable light chain region comprising an amino acid sequence of SEQ ID NO: 19. The anti-α-synuclein antibody, or antigen-binding fragment thereof, may comprise a variable heavy chain region comprising an amino acid sequence that is at least 95% identical to the amino acid sequence of SEQ ID NO: 14 and a variable light chain region comprising an amino acid sequence that is at least 95% identical to the amino acid sequence of SEQ ID NO: 19. The anti-α-synuclein antibody, or antigen-binding fragment thereof, may comprise a variable heavy chain region comprising the amino acid sequence of SEQ ID NO: 14 and a variable light chain region comprising the amino acid sequence of SEQ ID NO: 19.

The anti-α-synuclein antibody, or antigen-binding fragment thereof, may comprise a heavy chain comprising an amino acid sequence that is at least 90% identical to the amino acid sequence of SEQ ID NO: 12 or SEQ ID NO: 62. The anti-α-synuclein antibody, or antigen-binding fragment thereof, may comprise a heavy chain comprising the amino acid sequence of SEQ ID NO: 12 or SEQ ID NO: 62. The anti-α-synuclein antibody, or antigen-binding fragment thereof, may comprise a light chain comprising an amino acid sequence that is at least 90% identical to the amino acid sequence of SEQ ID NO: 17. The anti-α-synuclein antibody, or antigen-binding fragment thereof, may comprise a light chain comprising the amino acid sequence of SEQ ID NO: 17. The anti-α-synuclein antibody, or antigen-binding fragment thereof, may comprise a heavy chain comprising an amino acid sequence that is at least 95% identical to the amino acid sequence of SEQ ID NO: 12 or SEQ ID NO: 62 and a light chain comprising an amino acid sequence that is at least 95% identical to the amino acid sequence of SEQ ID NO: 17. The anti-α-synuclein antibody, or antigen-binding fragment thereof, may comprise a heavy chain comprising the amino acid sequence of SEQ ID NO: 12 or SEQ ID NO: 62 and a light chain comprising the amino acid sequence of SEQ ID NO: 17.

The anti-α-synuclein antibody, or antigen-binding fragment thereof, may be an antibody.

The anti-α-synuclein antibody, or antigen-binding fragment thereof, may comprise a triple mutation in the Fc region corresponding to L234F/L235E/P331S numbered based on the Kabat numbering.

### BRIEF DESRIPTION OF THE DRAWINGS

These and other features of the invention will become more apparent in the following detailed description in which reference is made to the appended drawings wherein:

FIG. 1 shows a simplified layout of a double-blind, placebo-controlled study of single ascending doses (SAD) of up to 4,500 mg of aslo0452 ngl-3 in healthy subjects aged 18 to 65 years.

FIG. 2 shows the observed aslo0452 ngl-3 pharmacokinetic (PK) concentrations in the single ascending dose trial, stratified by dose level.

FIG. 3 shows a simplified layout of a double-blind, placebo-controlled study of multiple ascending doses (MAD) of up to 4,200 mg of aslo0452 ngl-3 in subjects with Parkinson's Disease aged 40 to 85 years.

FIG. 4 shows the observed aslo0452 ngl-3 pharmacokinetic (PK) concentrations in the multiple ascending dose trial, stratified by dose level.

FIG. 5 shows the dose normalized (DN) observed aslo0452 ngl-3 pharmacokinetic (PK) concentrations in the single-and multiple-ascending dose trials.

Key to SEQ ID NOs:

| Name | Description | SEQ ID NO: |
|---|---|---|
| human alpha synuclein | amino acid sequence | 1 |
| asyn0087 | VH amino acid sequence | 2 |
| | VL amino acid sequence | 3 |
| General sequence | VH amino acid sequence | 4 |
| of an antibody | H-CDR1 amino acid sequence | 5 |
| derived from | H-CDR2 amino acid sequence | 6 |
| asyn0087 | H-CDR3 amino acid sequence | 7 |
| | VL amino acid sequence | 8 |
| | L-CDR1 amino acid sequence | 9 |
| | L-CDR2 amino acid sequence | 10 |
| | L-CDR3 amino acid sequence | 11 |
| aslo0452 | Heavy chain amino acid sequence | 12 or 62 |
| ngl-3 | VH nucleotide sequence | 13 |
| | VH amino acid sequence | 14 |
| | H-CDR1 amino acid sequence | 5 |
| | H-CDR2 amino acid sequence | 15 |
| | H-CDR3 amino acid sequence | 16 |
| | Light chain amino acid sequence | 17 |
| | VL nucleotide sequence | 18 |
| | VL amino acid sequence | 19 |
| | L-CDR1 amino acid sequence | 20 |
| | L-CDR2 amino acid sequence | 10 |
| | L-CDR3 amino acid sequence | 21 |
| aslo0543 | Heavy chain amino acid sequence | 22 |
| | VH nucleotide sequence | 23 |
| | VH amino acid sequence | 24 |
| | H-CDR1 amino acid sequence | 25 |
| | H-CDR2 amino acid sequence | 26 |
| | H-CDR3 amino acid sequence | 27 |
| | Light chain amino acid sequence | 28 |
| | VL nucleotide sequence | 29 |
| | VL amino acid sequence | 30 |
| | L-CDR1 amino acid sequence | 31 |
| | L-CDR2 amino acid sequence | 32 |
| | L-CDR3 amino acid sequence | 33 |
| General BBB | VH amino acid sequence | 34 |
| transporter | VL amino acid sequence | 35 |
| | L-CDR1 amino acid sequence | 36 |
| | L-CDR2 amino acid sequence | 37 |
| | L-CDR3 amino acid sequence | 38 |
| BBBt0626gl | VH amino acid sequence | 39 |
| | H-CDR1 amino acid sequence | 40 |
| | H-CDR2 amino acid sequence | 41 |
| | H-CDR3 amino acid sequence | 42 |
| | VL amino acid sequence | 43 |
| | L-CDR1 amino acid sequence | 44 |
| | L-CDR2 amino acid sequence | 45 |
| | L-CDR3 amino acid sequence | 46 |
| BBBt0626 | VH amino acid sequence | 47 |
| | H-CDR1 amino acid sequence | 40 |
| | H-CDR2 amino acid sequence | 41 |
| | H-CDR3 amino acid sequence | 42 |
| | VL amino acid sequence | 43 |
| | L-CDR1 amino acid sequence | 44 |
| | L-CDR2 amino acid sequence | 45 |
| | L-CDR3 amino acid sequence | 46 |
| BBBt0632gl | VH amino acid sequence | 48 |
| | H-CDR1 amino acid sequence | 49 |
| | H-CDR2 amino acid sequence | 50 |
| | H-CDR3 amino acid sequence | 51 |
| | VL amino acid sequence | 52 |
| | L-CDR1 amino acid sequence | 53 |
| | L-CDR2 amino acid sequence | 54 |
| | L-CDR3 amino acid sequence | 55 |
| Linker | GGGGS linker | 56 |
| Linker | GGGGS linker | 57 |
| Germline | Immunoglobulin heavy | 58 |
| IGHV3-23 | sequence | |
| JH6 | JH6 sequence | 59 |
| Germline | Immunoglobulin light | 60 |
| IGLV5-45 | sequence | |
| JL2 | JL2 sequence | 61 |

### DETAILED DESCRIPTION

### 4.1 Treatment and Prevention of α-synucleinopathies

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular instances only, and is not intended to limit the scope of the present invention, which is defined solely by the claims.

The invention provides a method for treating or preventing an α-synucleinopathy in a subject in need thereof, comprising administering to the subject a fixed dose of 50-5,000 mg of an anti-α-synuclein antibody, or antigen-binding fragment thereof. The invention also provides an anti-α-synuclein antibody, or antigen-binding fragment thereof, for use in a method of treating or preventing an α-synucleinopathy in a subject, the method comprising administering to the subject a fixed dose of 50-5,000 mg of the anti-α-synuclein antibody, or antigen-binding fragment thereof. The invention also provides use of an anti-α-synuclein antibody, or antigen-binding fragment thereof, in the manufacture of a medicament for treating or preventing an α-synucleinopathy in a subject, wherein the medicament comprises a fixed dose of 50-5,000 mg of said antibody or antigen-binding fragment thereof, or wherein said treating or preventing comprises administering a fixed dose of 50-5,000 mg of said antibody or antigen-binding fragment thereof. References herein to methods of the invention are also intended to encompass the equivalent uses of the invention, or antibodies of antigen binding fragments for use in the invention, or compositions comprising such antibodies of antigen binding fragments for use in the invention. Thus, where the invention relates to an anti-α-synuclein antibody, or antigen-binding fragment thereof, for use in a method of treating or preventing an α-synucleinopathy, or use of an anti-α-synuclein antibody or antigen-binding fragment thereof, in the manufacture of a medicament for treating or preventing an α-synucleinopathy, the method of treating or preventing an α-synucleinopathy may be any method for treating or preventing an α-synucleinopathy as described herein. Also provided is a kit comprising: (a) an anti-α-synuclein antibody, or antigen-binding fragment thereof; and (b) instructions for use of the same for treating an α-synucleinopathy at a fixed dose of 50-5,000 mg. The antibody, or antigen-binding fragment thereof, in the methods of the invention, the antibody or antigen-binding fragment thereof, for use in the invention, and the uses, compositions, or kits, of the invention may be any antibody, or antigen-binding fragment thereof, as described herein.

Preferably, the doses as disclosed herein are fixed doses. As used herein, the term “fixed dose” refers to a dose which is used for all subjects, e.g., the dose is an amount that does not vary based on the weight of the subject. A fixed dose may be a specific, unchanging amount, wherein the same dose is used for all subjects, or for all adult subjects, such as a unit dosage form as disclosed herein. A fixed dose is typically expressed in terms of an amount of the active agent, such as the antibody or antigen-binding fragment thereof, rather than an amount that is expressed relative to the weight or mass of the subject to be treated.

As used herein, the term “treating” or “treatment” refers to an amelioration of a disease or disorder, or at least one discernable symptom thereof. “Treatment” or “treating” may refer to an amelioration of at least one measurable physical parameter, not necessarily discernible by the patient. “Treatment” or “treating” may refer to inhibiting, reducing, or delaying the progression of a disease or disorder, either physically, e.g., stabilization of a discernible symptom, physiologically, e.g., stabilization of a physical parameter, or both. For example, “treating” or “treatment” may refer to reducing the progression of an α-synucleinopathy in a subject compared to the progression that would be expected for a subject having that α-synucleinopathy or compared to the progression that would be expected in a subject at that stage of the α-synucleinopathy. “Treatment” or “treating” may refer to delaying the onset of a disease or disorder.

As used herein, the term “preventing” or “prevention” refers to a reduction of the risk of acquiring a given disease or disorder. “Preventing” or “prevention” may refer to preventing or delaying the onset of one or more symptoms of a disease or disorder, e.g., compared to an untreated control or placebo.

“Subject” refers to an animal, such as a mammal, that has been or will be the object of treatment, observation, or experiment. The methods described herein may be useful for both human therapy and veterinary applications. Preferably, the subject is a human. The subject to be treated may be a human adult. The subject may be a human and may be at least 30 years old, at least 40 years old, at least 50 years old, at least 60 years old, at least 70 years old, or at least 75 years old. The subject may be at least 40 years old. The subject may be between 50 and 60 years old.

The subject to be treated may be a human having a BMI of at least 15 kg/m² at baseline. The subject may be a human having a BMI of at least 15, 16, 17, 18, 19, 20, 21, 22, 23, 24, 25, 26, 27, 28, 29, 30, 31, 32, 33, 34, 35, 36, 37, 38, 39 or 40 kg/m² at baseline. The subject may be a human having a BMI of less than or equal to 50 kg/m² at baseline. The subject may be a human having a BMI of less than or equal to 50, 49, 48, 47, 46, 45, 44, 43, 42, 41, or 40 kg/m² at baseline. The subject may be a human having a BMI of 15-40 kg/m² at baseline. Preferably, the subject is a human having a BMI of 18-35 kg/m² at baseline.

The methods, uses, compositions and kits of the invention utilise a dose (such as a fixed dose) of 50-5,000 mg of an anti-α-synuclein antibody, or antigen-binding fragment thereof, for example comprise administering such a (fixed) dose to a subject. The (fixed) dose of 50-5,000 mg may be at least 50 mg, such as at least 70 mg, at least 100 mg, at least 250 mg, at least 500 mg, at least 750 mg, at least 1,000 mg, at least 1,250 mg, at least 1,500 mg, at least 1,750 mg, at least 2,000 mg, at least 2,250 mg, at least 2,500 mg, at least 2,750 mg, at least 3,000 mg, at least 3,250 mg, at least 3,500 mg, at least 3,750 mg, at least 4,000 mg, at least 4,250 mg, at least 4,500 mg, or at least 4,750 mg. The (fixed) dose of 50-5,000 mg may be up to 5,000 mg, such as up to 4,750 mg, up to 4,500 mg, up to 4,250 mg, up to 4,000 mg, up to 3,750 mg, up to 3,500 mg, up to 3,250 mg, up to 3,000 mg, up to 2,750 mg, up to 2,500 mg, up to 2,250 mg, up to 2,000 mg, up to 1,750 mg, up to 1,500 mg, up to 1,250 mg, up to 1,000 mg, up to 750 mg, up to 500 mg, up to 250 mg, up to 100 mg to up to 70 mg. Any of these upper and lower end points may be combined within the range of 50 to 5,000 mg. For example, the anti-α-synuclein antibody, or antigen-binding fragment thereof, may be administered to the subject at a dose, such as a fixed dose, of 70-4,500 mg, 1,000-4,500 mg, 1,500-4,500 mg, 1,800-2,600 mg, 2,000-2,600 mg or 2,000-2,400 mg.

The anti-α-synuclein antibody, or antigen-binding fragment thereof, may be administered to the subject at a dose, such as a fixed dose, of up to 2,600 mg. For example, the anti-α-synuclein antibody, or antigen-binding fragment thereof, may be administered to the subject at a dose, such as a fixed dose, of 70-2,600 mg, 1,000-2,600 mg, 1,500-2,600 mg, 1,800-2,600 mg, or 2,000-2,600 mg.

The anti-α-synuclein antibody, or antigen-binding fragment thereof, may be administered to the subject at a dose, such as a fixed dose, of up to 2,400 mg. For example, the anti-α-synuclein antibody, or antigen-binding fragment thereof, may be administered to the subject at a dose, such as a fixed dose, of 70-2,400 mg, 1,000-2,400 mg, 1,500-2,400 mg, 1,800-2,400 mg, or 2,000-2,400 mg.

The anti-α-synuclein antibody, or antigen-binding fragment thereof, may be administered to the subject at a dose, such as a fixed dose, of up to 2,200 mg. For example, the anti-α-synuclein antibody, or antigen-binding fragment thereof, may be administered to the subject at a dose, such as a fixed dose, of 70-2,200 mg, 1,000-2,200 mg, 1,500-2,200 mg, 1,800-2,200 mg, or 2,000-2,200 mg.

The anti-α-synuclein antibody, or antigen-binding fragment thereof, may be administered to the subject at a dose, such as a fixed dose, of up to 2,000 mg. For example, the anti-α-synuclein antibody, or antigen-binding fragment thereof, may be administered to the subject at a dose, such as a fixed dose, of 70-2,000 mg, 1,000-2,000 mg, 1,500-2,000 mg, or 1,800-2,000 mg.

The anti-α-synuclein antibody, or antigen-binding fragment thereof, may be administered to the subject at a dose, such as a fixed dose, of 50, 100, 150, 200, 250, 300, 400, 500, 600, 700, 800, 900, 1,000, 1,200, 1,400, 1,600, 1,800, 2,000, 2,200, 2,400, 2,600, 2,800, 3,000, 3,200, 3,400, 3,600, 3,800, 4,000, 4,250, 4,500, 4,750 or 5,000 mg. The anti-α-synuclein antibody, or antigen-binding fragment thereof, may be administered to the subject at a dose, such as a fixed dose, of about 50, about 100, about 150, about 200, about 250, about 300, about 400, about 500, about 600, about 700, about 800, about 900, about 1,000, about 1,200, about 1,400, about 1,600, about 1,800, about 2,000, about 2,200, about 2,400, about 2,600, about 2,800, about 3,000, about 3,200, about 3,400, about 3,600, about 3,800, about 4,000, about 4,250, about 4,500, about 4,750 or about 5,000 mg. The anti-α-synuclein antibody, or antigen-binding fragment thereof, may be administered to the subject at a dose, such as a fixed dose, of about 70, 210, 400, 1,200, 2,000, 2,400, 4,200, or 4,500 mg. Preferably, the anti-α-synuclein antibody, or antigen-binding fragment thereof, is administered to the subject at a dose, such as a fixed dose, of about 1,200 mg, such as a dose of 1,200 mg. Preferably, the anti-α-synuclein antibody, or antigen-binding fragment thereof, is administered to the subject at a dose, such as a fixed dose, of about 2,000 mg, such as a dose of 2,000 mg. Preferably, the anti-α-synuclein antibody, or antigen-binding fragment thereof, is administered to the subject at a dose, such as a fixed dose, of about 2,400 mg, such as a dose of 2,400 mg. Preferably, the anti-α-synuclein antibody, or antigen-binding fragment thereof, is administered to the subject at a dose, such as a fixed dose, of about 4,200 mg, such as a dose of 4,200 mg. Preferably, the anti-α-synuclein antibody, or antigen-binding fragment thereof, is administered to the subject at a dose, such as a fixed dose, of about 4,500 mg, such as a dose of 4,500 mg.

The anti-α-synuclein antibody, or antigen-binding fragment thereof, may be administered to the subject at a dose, such as a fixed dose, of about 1,400 mg, such as a dose of 1,400 mg. The anti-α-synuclein antibody, or antigen-binding fragment thereof, may be administered to the subject at a dose, such as a fixed dose, of about 1,600 mg, such as a dose of 1,600 mg. The anti-α-synuclein antibody, or antigen-binding fragment thereof, may be administered to the subject at a dose, such as a fixed dose, of about 1,700 mg, such as a dose of 1,700 mg. The anti-α-synuclein antibody, or antigen-binding fragment thereof, may be administered to the subject at a dose, such as a fixed dose, of about 1,800 mg, such as a dose of 1,800 mg.

Treatment or prevention may be effected by a single administration or by multiple administrations of the anti-α-synuclein antibody or antigen-binding fragment thereof. For example, the treatment or prevention may comprise administering two doses, three doses, four doses, five doses, six doses, seven doses, eight doses, nine doses, ten doses, or more than ten doses of the anti-α-synuclein antibody, or antigen-binding fragment thereof, to the subject. Each dose may be a dose, such as a fixed dose, as disclosed herein. The treatment or prevention may comprise multiple administrations as described herein, such as repeated administrations, of the anti-α-synuclein antibody, or antigen-binding fragment thereof, over a course of treatment.

By a course of treatment is meant a treatment plan comprising several rounds of administration, as part of a method of treating or preventing an α-synucleinopathy in accordance with the invention. A course of treatment may last for one or more weeks, one or more months, such as two, three, four, five, six, seven, eight, nine, ten or eleven months, or for one or more years, such as one year, two years, three years, four years, five years, or longer. A course of treatment may be continued with no fixed end-point, for example a course of treatment may be continued for the lifetime of the subject being treated. Multiple administrations, such as repeated administrations, may be continued at regular or irregular intervals during the course of the treatment as determined to be needed by a medical practitioner.

The anti-α-synuclein antibody, or antigen-binding fragment thereof, may be administered to a subject daily or less frequently, for example, weekly or monthly. The anti-α-synuclein antibody, or antigen-binding fragment thereof, may be administered to a subject once per week, twice per week, three times per week, four times per week, five times per week, six times per week, or seven times per week. The anti-α-synuclein antibody, or antigen-binding fragment thereof, may be administered to a subject once per two weeks, once per three weeks, once per four weeks, once per month, twice per month, three times per month or four times per month. The anti-α-synuclein antibody, or antigen-binding fragment thereof, may be administered to a subject once per year, twice per year, three times per year, four times per year, five times per year, six times per year, seven times per year, eight times per year, nine times per year, ten times per year, eleven times per year, or twelve times per year.

The anti-α-synuclein antibody, or antigen-binding fragment thereof, may be administered to a subject multiple times, at an interval of one week, two weeks, three weeks, four weeks, one month, two months, three months, six months, or one year. The treatment or prevention may comprise administering the anti-α-synuclein antibody, or antigen-binding fragment thereof, to a subject every week, every two weeks, every three weeks, every three to five weeks, every four weeks, every month, every two months, every three months, every six months, or every year.

When the anti-α-synuclein antibody, or antigen-binding fragment, thereof is administered in multiple administrations, such as repeated administrations, each of said administrations may be at a fixed dose as described herein. For example, each administration in a method or use of the invention may comprise a fixed dose of 50-5,000 mg of an anti-α-synuclein antibody, or antigen-binding fragment thereof, as disclosed herein. In such instances, the number of administrations or the frequency of administrations may be any of the options disclosed herein. The multiple administrations, such as repeated administrations, of the anti-α-synuclein antibody, or antigen-binding fragment thereof, may be each at the same dose. Two or more administrations of the anti-α-synuclein antibody, or antigen-binding fragment thereof, may be each at the same dose. The multiple administrations, such as repeated administrations of the anti-α-synuclein antibody, or antigen-binding fragment thereof, may be each at a dose within a range of doses disclosed herein. For example, each administration of the anti-α-synuclein antibody, or antigen-binding fragment thereof, in a method or use of the invention may be at a fixed dose in the range of 70-4,500 mg. Each administration of the anti-α-synuclein antibody, or antigen-binding fragment thereof, in a method or use of the invention may be at a fixed dose in the range of 1,000-4,500 mg. Each administration of the anti-α-synuclein antibody, or antigen-binding fragment thereof, in a method or use of the invention may be at a fixed dose in the range of 1,800-2,600 mg. Each administration of the anti-α-synuclein antibody, or antigen-binding fragment thereof, in a method or use of the invention may be at a fixed dose in the range of 2,000-2,400 mg. Each administration of the anti-α-synuclein antibody, or antigen-binding fragment thereof, in a method or use of the invention may be at a fixed dose of about 2,000 mg. Each administration in a method or use of the invention may be at a fixed dose of about 2,400 mg.

Each administration in a method or use of the invention may comprise a fixed dose of up to 2,600 mg of an anti-α-synuclein antibody, or antigen-binding fragment thereof, as disclosed herein. For example, each administration of the anti-α-synuclein antibody, or antigen-binding fragment thereof, in a method or use of the invention may be at a fixed dose in the range of 70-2,600 mg. Each administration of the anti-α-synuclein antibody, or antigen-binding fragment thereof, in a method or use of the invention may be at a fixed dose in the range of 1,000-2,600 mg. Each administration of the anti-α-synuclein antibody, or antigen-binding fragment thereof, in a method or use of the invention may be at a fixed dose in the range of 1,500-2,600 mg. Each administration of the anti-α-synuclein antibody, or antigen-binding fragment thereof, in a method or use of the invention may be at a fixed dose in the range of 2,000-2,600 mg.

Each administration in a method or use of the invention may comprise a fixed dose of up to 2,400 mg of an anti-α-synuclein antibody, or antigen-binding fragment thereof, as disclosed herein. For example, each administration of the anti-α-synuclein antibody, or antigen-binding fragment thereof, in a method or use of the invention may be at a fixed dose in the range of 70-2,400 mg. Each administration of the anti-α-synuclein antibody, or antigen-binding fragment thereof, in a method or use of the invention may be at a fixed dose in the range of 1,000-2,400 mg. Each administration of the anti-α-synuclein antibody, or antigen-binding fragment thereof, in a method or use of the invention may be at a fixed dose in the range of 1,500-2,400 mg. Each administration of the anti-α-synuclein antibody, or antigen-binding fragment thereof, in a method or use of the invention may be at a fixed dose in the range of 2,000-2,400 mg.

Each administration in a method or use of the invention may comprise a fixed dose of up to 2,200 mg of an anti-α-synuclein antibody, or antigen-binding fragment thereof, as disclosed herein. For example, each administration of the anti-α-synuclein antibody, or antigen-binding fragment thereof, in a method or use of the invention may be at a fixed dose in the range of 70-2,200 mg. Each administration of the anti-α-synuclein antibody, or antigen-binding fragment thereof, in a method or use of the invention may be at a fixed dose in the range of 1,000-2,200 mg. Each administration of the anti-α-synuclein antibody, or antigen-binding fragment thereof, in a method or use of the invention may be at a fixed dose in the range of 1,500-2,200 mg. Each administration of the anti-α-synuclein antibody, or antigen-binding fragment thereof, in a method or use of the invention may be at a fixed dose in the range of 2,000-2,200 mg.

Each administration in a method or use of the invention may comprise a fixed dose of up to 2,000 mg of an anti-α-synuclein antibody, or antigen-binding fragment thereof, as disclosed herein. For example, each administration of the anti-α-synuclein antibody, or antigen-binding fragment thereof, in a method or use of the invention may be at a fixed dose in the range of 70-2,000 mg. Each administration of the anti-α-synuclein antibody, or antigen-binding fragment thereof, in a method or use of the invention may be at a fixed dose in the range of 1,000-2,000 mg. Each administration of the anti-α-synuclein antibody, or antigen-binding fragment thereof, in a method or use of the invention may be at a fixed dose in the range of 1,500-2,000 mg.

Each administration of the anti-α-synuclein antibody, or antigen-binding fragment thereof, in a method or use of the invention may be at a fixed dose of about 1,400 mg. Each administration of the anti-α-synuclein antibody, or antigen-binding fragment thereof, in a method or use of the invention may be at a fixed dose of about 1,600 mg. Each administration of the anti-α-synuclein antibody, or antigen-binding fragment thereof, in a method or use of the invention may be at a fixed dose of about 1,700 mg. Each administration of the anti-α-synuclein antibody, or antigen-binding fragment thereof, in a method or use of the invention may be at a fixed dose of about 1,800 mg.

The anti-α-synuclein antibody, or antigen-binding fragment thereof, may be administered to a subject every four weeks. Accordingly, the method or use of the invention may comprise administering to the subject a fixed dose of 2,000, 2,400 or 4,500 mg of the anti-α-synuclein antibody, or antigen-binding fragment thereof, every four weeks. The method may comprise administering to the subject a fixed dose of 2,000 mg of the anti-α-synuclein antibody, or antigen-binding fragment thereof, every four weeks. The method may comprise administering to the subject a fixed dose of 2,400 mg of the anti-α-synuclein antibody, or antigen-binding fragment thereof, every four weeks. The method may comprise administering to the subject a fixed dose of 4,500 mg of the anti-α-synuclein antibody, or antigen-binding fragment thereof, every four weeks.

The method may comprise administering to the subject a fixed dose of 1,400 mg of the anti-α-synuclein antibody, or antigen-binding fragment thereof, every four weeks. The method may comprise administering to the subject a fixed dose of 1,600 mg of the anti-α-synuclein antibody, or antigen-binding fragment thereof, every four weeks. The method may comprise administering to the subject a fixed dose of 1,700 mg of the anti-α-synuclein antibody, or antigen-binding fragment thereof, every four weeks. The method may comprise administering to the subject a fixed dose of 1,800 mg of the anti-α-synuclein antibody, or antigen-binding fragment thereof, every four weeks.

In accordance with the invention, the anti-α-synuclein antibody, or antigen-binding fragment thereof, may be administered to a subject multiple times over a period of at least 3 months, 6 months, 9 months, 1 year, 2 years or 5 years. The anti-α-synuclein antibody, or antigen-binding fragment thereof, may be administered to a subject twice every month over a period of at least 3 months, 6 months, 9 months, 1 year, 2 years or 5 years. The anti-α-synuclein antibody, or antigen-binding fragment thereof, may be administered to a subject once every month over a period of at least 3 months, 6 months, 9 months, 1 year, 2 years or 5 years. Preferably, the anti-α-synuclein antibody, or antigen-binding fragment thereof, is administered to a subject once every four weeks over a period of at least 3 months, 6 months, 9 months, 1 year, 2 years or 5 years. Particularly preferably, the anti-α-synuclein antibody, or antigen-binding fragment thereof, is administered to a subject once every four weeks over a period of at least 1 year. Accordingly, the method may comprise administering to the subject a fixed dose of 1,200, 2,000, 2,400, 4,200 or 4,500 mg of the anti-α-synuclein antibody, or antigen-binding fragment thereof, once every four weeks over a period of at least 1 year. The method may comprise administering to the subject a fixed dose of 1,200 mg of the anti-α-synuclein antibody, or antigen-binding fragment thereof, once every four weeks over a period of at least 1 year. The method may comprise administering to the subject a fixed dose of 2,000 mg of the anti-α-synuclein antibody, or antigen-binding fragment thereof, once every four weeks over a period of at least 1 year. The method may comprise administering to the subject a fixed dose of 2,400 mg of the anti-α-synuclein antibody, or antigen-binding fragment thereof, once every four weeks over a period of at least 1 year. The method may comprise administering to the subject a fixed dose of 4,200 mg of the anti-α-synuclein antibody, or antigen-binding fragment thereof, once every four weeks over a period of at least 1 year. The method may comprise administering to the subject a fixed dose of 4,500 mg of the anti-α-synuclein antibody, or antigen-binding fragment thereof, once every four weeks over a period of at least 1 year.

The method may comprise administering to the subject a fixed dose of 1,400 mg of the anti-α-synuclein antibody, or antigen-binding fragment thereof, once every four weeks over a period of at least 1 year. The method may comprise administering to the subject a fixed dose of 1,600 mg of the anti-α-synuclein antibody, or antigen-binding fragment thereof, once every four weeks over a period of at least 1 year. The method may comprise administering to the subject a fixed dose of 1,700 mg of the anti-α-synuclein antibody, or antigen-binding fragment thereof, once every four weeks over a period of at least 1 year. The method may comprise administering to the subject a fixed dose of 1,800 mg of the anti-α-synuclein antibody, or antigen-binding fragment thereof, once every four weeks over a period of at least 1 year.

The anti-α-synuclein antibody or antigen-binding fragment thereof may be administered via any suitable route. For example, the anti-α-synuclein antibody, or antigen-binding fragment thereof, may be administered intravenously, intraperitoneally, transdermally, intracavitary, subcutaneously, intradermally or intramuscularly. Preferably, the anti-α-synuclein antibody, or antigen-binding fragment thereof, is administered intravenously.

As used herein, and in accordance with the invention, the term “α-synucleinopathy” or “alpha-synucleinopathy” refers to a neurodegenerative disease characterized by abnormal accumulation of insoluble α-synuclein in neurons and glial cells in a subject. The term “α-synucleinopathy” is discussed further in Mendoza-Velásquez et al. (2019) Front. Neurol. 10: 363), which is hereby incorporated by reference in its entirety. The α-synucleinopathy may be selected from Parkinson's disease (PD), dementia with Lewy bodies (DLB), multiple system atrophy (MSA). The α-synucleinopathy may be selected from Alzheimer's disease, pure autonomic failure and REM behavior disorder. The α-synucleinopathy may be a prodromal synucleinopathy, for example, characterized by an early sign or symptom of an α-synucleinopathy, such as REM behavior disorder or autonomic symptoms such as orthostatic hypotension. Preferably, the α-synucleinopathy is PD or MSA.

The α-synucleinopathy may be Parkinson's disease (PD). The subject to be treated in accordance with the present invention may have PD. The subject may have been diagnosed with PD. The subject may have one or more symptoms of PD, such as one or more of tremor, bradykinesia, postural instability, and rigid muscles. The subject may have a diagnosis of PD based on the Movement Disorder Society (MDS) Clinical Diagnostic Criteria for Parkinson's disease (see, e.g., Postuma et al. (2015) Move.Disord. 30: 1591-1599). The subject may be over 50 years of age, such as over 55, over 60, over 65, over 70, or over 75 years of age. The subject may have early onset PD. The subject may be under 50 years of age. The subject may be at risk of PD. The subject may be predisposed to PD, e.g., has a family history of PD, has a genetic predisposition to PD, and/or has been exposed to environmental factors linked to PD.

Preferably, the subject may have early PD. The subject may have been diagnosed with early PD. The subject may have a diagnosis of early-stage PD. The subject may have one, two, three, four, five, six, seven, eight or more symptoms of early PD. Preferably, the subject has two or more symptoms of early PD. More preferably, the subject has two or more symptoms of early PD selected from tremor, slowing of movement, and muscle stiffness. Symptoms of early PD may include one or more of: a) smaller handwriting; b) tremors; c) muscle stiffness; d) slowing of movement; e) stooped posture; f) lack of facial expression; g) decreased arm swing; and h) soft or low voice.

The α-synucleinopathy may be pure autonomic failure. Pure autonomic failure is a neurodegenerative disorder of the autonomic nervous system clinically characterized by orthostatic hypotension. Pure autonomic failure is described in more detail in Coon et al. (2019) Mayo Clin. Proc. 94(10): 2087-2098.

The α-synucleinopathy may be REM behavior disorder. REM behavior disorder (RBD) involves REM sleep without atonia in conjunction with a recurrent nocturnal dream enactment behavior, with vocalizations such as shouting and screaming, and motor behaviors such as punching and kicking. RBD is described in more detail in Barone and Henchcliffe (2018) Clin. Neurophysiol. 129(8): 1551-1564.

The α-synucleinopathy may be MSA. The subject to be treated in accordance with the present invention may have MSA. The subject may have been diagnosed with MSA. The subject may have one or more symptoms of MSA. The subject may be at risk of MSA. The subject may be predisposed to MSA. The subject may be at least 30 years old, at least 40 years old, at least 50 years old, at least 60 years old or at least 70 years old. The subject may be at least 40 years old. The subject may be between 50 and 60 years old.

Preferably, the subject has early MSA. The subject may have been diagnosed with early MSA. The subject may have a diagnosis of early-stage MSA. The subject may have one, two, three, four or five or more symptoms of early MSA. Preferably, the subject has two or more symptoms of early MSA. More preferably, the subject has two or more symptoms of early PD selected from tremor, slowing of movement, and muscle stiffness. Symptoms of early MSA may include one or more of: a) slowness of movement, tremor, or rigidity (stiffness); b) clumsiness or incoordination; c) impaired speech, a croaky, quivering voice; d) fainting or lightheadedness due to orthostatic hypotension; and e) bladder control problems, such as a sudden urge to urinate or difficulty emptying the bladder.

The subject may have a diagnosis of possible or probable MSA. As used herein, the term “possible or probable MSA” means possible or probable MSA defined using the modified Gilman et al., 2008 diagnostic criteria described in detail in Gilman et al. (2008) Neurol. 71(9): 670-676.

The criteria for a diagnosis of possible MSA are as follows:

(1) A sporadic, progressive, adult (over 30 years old)-onset disease characterized by the following:
a) Parkinsonism (bradykinesia with rigidity, tremor, or postural instability); or a cerebellar syndrome (gait ataxia with cerebellar dysarthia, limb ataxia, or cerebellar oculomotor dysfunction), and
b) At least 1 feature suggesting autonomic dysfunction (otherwise unexplained urinary urgency, frequency or incomplete bladder emptying, erectile dysfunction in male subjects, or significant orthostatic blood pressure decline that does not meet the level required for improbable MSA), and
   (2) At least 1 of the following additional features:
   For possible MSA with predominant parkinsonism (MSA-P) or MSA with predominant cerebellar ataxia (MSA-C):
a) Babinski sign with hyperreflexial; and/or
b) Stridor.
   For possible MSA with predominant parkinsonism (MSA-P):
a) Rapidly progressive parkinsonism;
b) Poor response to levodopa;
c) Postural instability within 3 years of motor onset;
d) Gait ataxia, cerebellar dysarthria, limb ataxia or cerebellar oculomotor dysfunction;
e) Dysphagia within 5 years of motor onset;
f) Atrophy on MRI of the putamen, middle cerebellar peduncle, pons, or cerebellum; and/or
g) Hypometabolism on fluorodeoxyglucose (FDG)-positron emission tomography (PET) in the putamen, brainstem, or cerebellum.
   For possible MSA with predominant cerebellar ataxia (MSA-C):
a) Parkinsonism (bradykinesia and rigidity);
b) Atrophy on MRI of the putamen, middle cerebellar peduncle, or pons;
c) Hypometabolism on FDG-PET in the putamen; and/or
d) Presynaptic nigrostriatal dopaminergic denervation on single-photon emission computerized tomography or PET.

The criteria for a diagnosis of probable MSA are as follows:

A sporadic, progressive, adult (over 30 years old)-onset disease characterized by the following:
a) Autonomic failure involving urinary incontinence (inability to control the release of urine from the bladder, with erectile dysfunction in male subjects) or an orthostatic decrease of blood pressure within 3 minutes of standing by at least 30 mm Hg systolic or 15 mm Hg diastolic; and either
b) Poorly levodopa-responsive parkinsonism (bradykinesia with rigidity, tremor, or postural instability), or
c) A cerebellar syndrome (gait ataxia with cerebellar dysarthria, limb ataxia, or cerebellar oculomotor dysfunction).

The subject may have a diagnosis of “clinically established MSA”, “clinically probable MSA” or “possible prodromal MSA”. As used herein, the terms “clinically established MSA”, “clinically probable MSA” and “possible prodromal MSA” means clinically established MSA, clinically probable MSA and possible prodromal MSA respectively, as defined in Wenning et al. (2022) Mov. Disor. 37(6): 1131-1148.

The criteria for a diagnosis of clinically established MSA are as follows:

(1) a sporadic, progressive adult (over 30 years old) onset disease characterized by the following:
at least one of:
   (a) unexplained voiding difficulties with post-void urinary residual volume ≥100 mL;
   (b) unexplained urinary urge incontinence; and
   (c) neurogenic OH (>20/10 mmHg blood pressure drop) within 3 minutes of standing or head-up tilt test;
and at least one of:
   (a) poorly L-dopa-responsive parkinsonism; and
   (b) cerebellar syndrome (at least two of gait ataxia, limb ataxia, cerebellar dysarthria,
   or oculomotor features; and
      (2) at least two supportive clinical (motor or non-motor) features selected from:
(a) rapid progression within 3 years of motor onset;
(b) moderate to severe postural instability within 3 years of motor onset;
(c) craniocervical dystonia induced or exacerbated by L-dopa in the absence of limb dyskinesia;
(d) severe speech impairment within 3 years of motor onset;
(e) severe dysphagia within 3 years of motor onset;
(f) unexplained Babinski sign;
(g) jerky myoclonic postural or kinetic tremor;
(h) postural deformities;
(i) stridor;
(j) inspiratory sighs;
(k) cold discolored hands and feet; and
(I) pathologic laughter or crying; and
   (3) at least one MRI marker selected from the following (each affected brain region as evidenced by either atrophy or increased diffusivity counts as one MRI marker):

For MSA-P:
(a) atrophy of:
   (i) putamen (and signal decrease on iron-sensitive sequences);
   (ii) middle cerebellar peduncle;
   (iii) pons; or
   (iv) cerebellum;
(b) “hot cross bun” sign; and
(c) increase diffusivity of:
   (i) putamen; or
   (ii) middle cerebellar peduncle;

For MSA-C:
(a) atrophy of:
   (i) putamen (and signal decrease on iron-sensitive sequences); or
   (ii) infratentorial structures (pons and middle cerebellar peduncle);
(b) “hot cross bun” sign; and
(c) increased diffusivity of putamen; and
   (4) absence of all of the following features:
(a) substantial and persistent beneficial response to dopaminergic medications;
(b) unexplained anosmia on olfactory testing;
(c) fluctuating cognition with pronounced variation in attention and alertness and early decline in visuoperceptual abilities;
(d) recurrent visual hallucinations not induced by drugs within 3 years of disease onset;
(e) dementia according to DSM-V within 3 years of disease onset;
(f) downgaze supranuclear palsy or slowing of vertical saccades;
(g) brain MRI findings suggesting of an alternative diagnosis (e.g. PSP, multiple sclerosis, vascular parkinsonism, symptomatic cerebellar disease, etc.); and
(h) documentation of alternative condition (MSA look-alike, including genetic or symptomatic ataxia and parkinsonism) known to produce autonomic failure, ataxia, or parkinsonism and plausible coonected to the patient's symptoms.

The criteria for a diagnosis of clinically probable MSA are as follows:

(1) a sporadic, progressive adult (over 30 years old) onset disease characterized by the following:
at least two of:
   (a) autonomic dysfunction defined as:
      (i) unexplained voiding difficulties with post-void urinary residual volume;
      (ii) unexplained urinary urge incontinence; and
      (iii) neurogenic OH (≥20/10 mmHg blood pressure drop) within 10 minutes of standing or head-up tilt test;
   (b) parkinsonism; and
   (c) cerebellar syndrome (defined as at least one of gait ataxia, limb ataxia, cerebellar dysarthria, and oculomotor features);
      (2) at least one supportive clinical (motor or non-motor) feature selected from:
(a) rapid progression within 3 years of motor onset;
(b) moderate to severe postural instability within 3 years of motor onset;
(c) craniocervical dystonia induced or exacerbated by L-dopa in the absence of limb
dyskinesia;
(d) severe speech impairment within 3 years of motor onset;
(e) severe dysphagia within 3 years of motor onset;
(f) unexplained Babinski sign;
(g) jerky myoclonic postural or kinetic tremor;
(h) postural deformities;
(i) stridor;
(j) inspiratory sighs;
(k) cold discolored hands and feet; and
(l) pathologic laughter or crying; and
   (3) absence of all of the following features:
(a) substantial and persistent beneficial response to dopaminergic medications;
(b) unexplained anosmia on olfactory testing;
(c) fluctuating cognition with pronounced variation in attention and alertness and early decline in visuoperceptual abilities;
(d) recurrent visual hallucinations not induced by drugs within 3 years of disease onset;
(e) dementia according to DSM-V within 3 years of disease onset;
(f) downgaze supranuclear palsy or slowing of vertical saccades;
(g) brain MRI findings suggesting of an alternative diagnosis (e.g. PSP, multiple sclerosis, vascular parkinsonism, symptomatic cerebellar disease, etc.); and
(h) documentation of alternative condition (MSA look-alike, including genetic or symptomatic ataxia and parkinsonism) known to produce autonomic failure, ataxia, or parkinsonism and plausible coonected to the patient's symptoms.

The criteria for a diagnosis of possible prodromal MSA are as follows:

(1) a sporadic, progressive adult (over 30 years old) onset disease characterized by the following:
at least one of:
   (a) RBD (polysomnography proven);
   (b) neurogenic OH (≥20/10 mmHg blood pressure drop) within 10 minutes of standing or head-up tilt test; and (
   c) urogenital failure (erectile dysfunction in males below age of 60 years combined with at least one of unexplained voiding difficulties with post-void urinary residual volume >100 mL and unexplained urinary urge incontinence); and
      (2) at least one of the following:
(a) subtle parkinsonian signs; and
(b) subtle cerebellar signs; and
   (3) absence of all of the following:
(a) at least one of unexplained anosmia on olgactory testing or abnormal cardiac sympathetic imagining (¹²³I-MIBG-scintigraphy);
(b) fluctuating cognition with pronounced variation in attention and alertness and early decline in visuoperceptual abilities;
(c) recurrent visual hallucinations not induced by drugs within 3 years of disease onset;
(d) dementia according to DSM-V within 3 years of disease onset;
(e) downgaze supranuclear gaze palsy or slowing of vertical saccades;
(f) brain MRI findings suggesting of an alternative diagnosis (e.g. PSP, multiple sclerosis, vascular parkinsonism, symptomatic cerebellar disease, etc.); and
(g) documentation of an alternative conditions (MSA look-alike, including genetic or symptomatic ataxia and parkinsonism) known to produce autonomic failure, ataxia, or parkinsonism and plausibly connected to the patient's symptoms.

The subject to be treated in accordance with the present invention may be defined or selected based on scoring in the Unified Multiple System Atrophy Rating Scale. The subject may have a Unified Multiple System Atrophy Rating Scale (UMSARS) Part I score of 40 or less. The subject may have a Unified Multiple System Atrophy Rating Scale (UMSARS) Part I score of 30 or less. Preferably, the subject has a Unified Multiple System Atrophy Rating Scale (UMSARS) Part I score of 24 or less. The UMSARS score (described in more detail in Wenning et al. (2004) Mov. Disord. 19(12): 1391-1402) is a clinical rating scale designed to assess the severity of MSA in a subject with higher scores indicating more severe disease. The UMSARS comprises four subscales: UMSARS Part I (12 questions) rates patient-reported functional disability; UMSARS Part II (14 questions) rates clinician-assessed motor impairment; UMSARS Part III records blood pressure and heart rate measurements in the supine and standing positions; and UMSARS Part IV (1 question) rates chore-based disability. The answers to the questions or items of UMSARS Part I and Part II are scored from 0-4 with a score of 0 indicating normal function and a score of 1 indicating mild disability. The subject may have a severity score of 2 or less on the swallowing item. The subject may have a severity score of 2 or less on the ambulation item. The subject may have a severity score of 2 or less on the falling item. The subject may have a UMSARS Part IV disability score of 3 or less. The subject may have a UMSARS Part I score of 21 or less. Preferably, where these scores are used herein to define the subject that is to be treated in accordance with the invention, they refer to the score of the subject at baseline, i.e., the score of the subject immediately before initiation of treatment with the anti-α-synuclein antibody or antigen-binding fragment thereof.

The subject to be treated (e.g., at baseline) may have a Montreal Cognitive Assessment (MoCA) score of MoCA) ≥18. The MoCA test was developed to detect mild cognitive impairment but is now frequently used as a screening tool for the dementias. The MoCA is scored out of 30 points and is a brief test of cognitive function, taking 10 minutes to administer. It assesses short-term memory, visuospatial function, executive function, attention, concentration and working memory, language and orientation. The MoCA test is described in more detail in Nasreddine et al. (2005) J. Amer. Geriatrics Soc. 53(4): 659-9.

### 4.1.1 Clinical Outcomes

The present invention relates to treating or preventing an α-synucleinopathy. Treatment or prevention may be observed as a reduction in one or more markers and/or symptoms of the α-synucleinopathy, and/or a reduction in the score(s) from one or more relevant scoring systems for the α-synucleinopathy. For example, in a subject treated in accordance with the invention, the subject's condition may improve. In a subject treated in accordance with the invention, one or more symptoms and/or markers and/or scores of the α-synucleinopathy may improve. Treatment or prevention may be observed as a reduction in the progression of the α-synucleinopathy in the subject or a delay in the progression of the α-synucleinopathy in the subject. For example, in a subject treated in accordance with the invention, the subject's condition may stabilise or remain stable. In a subject treated in accordance with the invention, one or more symptoms and/or markers and/or scores of the α-synucleinopathy may remain stable, may stabilise, or may not deteriorate. In a subject treated in accordance with the invention, the α-synucleinopathy may continue to progress, but at a slower rate than would be expected for a subject having that type of α-synucleinopathy or for a subject at that stage of the disease. Progression of an α-synucleinopathy may be measured by assessing one or more markers and/or symptoms and/or scores of the α-synucleinopathy at two or more time points in a subject (e.g., before treatment in accordance with the invention (e.g., at baseline) and during and/or after treatment) and determining whether there has been any change over that period of time. A reduction or delay in progression may be measured by comparing the progression in a subject treated with an antibody, or antigen-binding fragment thereof, as described herein, with the change in the same markers and/or symptoms and/or scores over the same period of time in a subject not treated with such an antibody, or antigen-binding fragment thereof (e.g., a control or placebo-treated subject or a group of controls or group of placebo-treated subjects). Preferably the comparison is with a group of placebo treated subjects, such as a typical or average change in score obtained from a group of placebo-treated subjects. Preferably the control or placebo-treated subject(s) are subjects having the same disease or disorder as the subject being treated. Preferably the control or placebo treated subject(s) have the same or similar level of disease progression as the subject being treated at the start of treatment. Preferably the control or placebo treated subjects(s) have the same scope or a similar score to the subject being treated at the start of treatment. A reduction or delay in progression may be measured by comparing the progression in a subject treated with an antibody, or antigen-binding fragment thereof, as described herein with the change in the same markers and/or symptoms and/or scores that would be expected or predicted over the same period of time in a subject not treated with such an antibody, or antigen-binding fragment thereof, e.g., based on models of disease progression, e.g., the normal level or average level of progression in reference subject(s) having the same disease or disorder or the same α-synucleinopathy as the subject, or a reference subject having the same degree of disease progression as the subject being treated.

Treating or preventing in the present invention may be observed as such a decrease, stabilisation or delayed or reduced progression in (i) α-synuclein spreading in a subject; (ii) α-synuclein levels in the cerebrospinal fluid (CSF) of a subject; and/or (iii) α-synuclein levels in the brain interstitial fluid (ISF) of a subject. Accordingly, the method may reduce progression of the α-synucleinopathy as measured by α-synuclein spreading in the subject; free unbound α-synuclein levels in the CSF of the subject; and/or free unbound α-synuclein levels in the ISF of the subject. The method may reduce progression of the α-synucleinopathy as measured by α-synuclein spreading in the subject and free unbound α-synuclein levels in the CSF of the subject. The method may reduce progression of the α-synucleinopathy as measured by α-synuclein spreading in the subject. Preferably, the method reduces progression of the α-synucleinopathy as measured by free unbound α-synuclein levels in the CSF of the subject. The method may reduce progression of the α-synucleinopathy as measured by free unbound α-synuclein levels in the brain interstitial fluid of the subject. Advantageously, these effects may be obtained by administering the anti-α-synuclein antibody, or antigen-binding fragment thereof, intravenously.

The methods disclosed herein may provide an advantage over other therapeutics by reducing α-synuclein spreading in a subject; reducing α-synuclein levels in the CSF of a subject; and/or by reducing α-synuclein levels in the brain interstitial flid of a subject. Accordingly, the method may reduce α-synuclein spreading in the subject; reduce free unbound α-synuclein levels in the CSF of the subject; and/or reduce free unbound α-synuclein levels in the ISF of the subject. The method may reduce α-synuclein spreading in the subject and reduce free unbound α-synuclein levels in the CSF of the subject. The method may reduce α-synuclein spreading in the subject. Preferably, the method reduces free unbound α-synuclein levels in the cerebrospinal fluid (CSF) of the subject. The method may reduce free unbound α-synuclein levels in the brain interstitial fluid (ISF) of the subject. Advantageously, the anti-α-synuclein antibody, or antigen-binding fragment thereof, may reduce α-synuclein spreading, reduce free unbound α-synuclein levels in the cerebrospinal fluid and/or reduce free unbound α-synuclein levels in the brain interstitial fluid when administered intravenously.

Alpha-synuclein spreading in a subject can be measured by any suitable means. Alpha-synuclein is abundant in the central nervous system (CNS) and brain where it is found both intracellularly in neurons and glia and also extracellularly in cerebrospinal fluid (CSF) (Mollenhauer et al. (2012) J. Neural. Transm. (2012), 119(7): 739-46; incorporated herein by reference) and the interstitial fluid (ISF) that bathes and surrounds the cells of the brain (Emmanouilidou et al. (2011) PLOS One (2011), 6(7): e22225; incorporated herein by reference). Alpha-synuclein is a synaptic protein predominantly expressed in neurons of the neocortex, hippocampus, substantia nigra, thalamus, and cerebellum (Iwai et al. (1995) Neuron (1995), 14: 467-475; incorporated herein by reference). Under physiological conditions, it is located in neuronal synaptic terminals and is specifically up-regulated at presynaptic terminals during acquisition-related synaptic rearrangement (Fortin et al. (2005) J. Neurosci. (2005), 25: 10913-10921; incorporated herein by reference). A variety of different experimental models have demonstrated cell-to-cell transmission of alpha-synuclein in cultured cells, or in vivo spreading and propagation of alpha-synuclein pathologies. For example, preformed recombinant alpha-synuclein fibrils and alpha-synuclein oligomers can be internalised by cultured cells and neurons, and the direct transfer of alpha-synuclein from donor to recipient cells with the formation of alpha-synuclein inclusions similar to Lewy pathology has been demonstrated (Danzer et al. (2007) J. Neurosci. (2007), 27(34): 9220-32; Volpicelli-Daley et al. (2011) Neuron (2011), 72(1): 57-71; and Luk et al. (2009) Proc. Natl. Acad. Sci. U S A (2009), 106(47): 20051-6; each of which are incorporated herein by reference). In addition, transfer and transmission of both monomeric and oligomeric alpha-synuclein from the olfactory bulb to interconnected brain structures has been demonstrated in mice (Rey et al. (2013) Acta Neuropathol. (2013), 126(4): 555-73; incorporated herein by reference).

As used herein, the term “α-synuclein spreading” refers to an expansion of the area in which α-synuclein is found. This may relate to an expansion of the area in which misfolded α-synuclein, or aggregated α-synuclein, such as Lewy bodies or Lewy body like aggregates, is found. α-synuclein spreading may involve the spreading of α-synuclein to a wider area of the brain or to additional areas of the brain. α-synuclein spreading may involve the spreading of α-synuclein to a wider area of the periphery or to additional areas of the periphery. α-synuclein spreading may involve the movement of α-synuclein via cell-to-cell transmission. For example, α-synuclein oligomers can be released to the extracellular environment and taken up by neighboring cells in a “propagation” mechanism (Angot and Brundin (2009) Parkinsonism Relat. Disord. 15 Suppl 3: S143-147; Desplats et al. (2009) Proc. Natl. Acad. Sci. USA 106: 13010-13015; and Lee et al. (2010) J. Biol. Chem. 285: 9262-9272). α-synuclein spreading may involve the spreading of misfolded α-synuclein, such as α-synuclein oligomers or polymers or other forms of aggregated α-synuclein, such as the formation or spreading of Lewy bodies or Lewy body like aggregates, to a wider area of the brain or to additional areas of the brain. For example, this may occur by the propagation of α-synuclein misfolding through a prion-like spreading mechanism (Lee et al. (2010) Nat. Rev. Neurol. 6: 702-706; Luk et al. (2012) J. Exp. Med. 209(5): 975-86; and Luk et al. (2012) Science 338(6109): 949-54). The α-synuclein spreading may occur within a single area of the brain and/or may involve spreading to a different area of the brain, such as to an interconnected or a neighboring brain region. Preferably, the invention leads to a reduction in α-synuclein spreading in the subject. A reduction in α-synuclein spreading may comprise a reduction in the extent of spreading in the subject compared to that seen in a control subject that is not administered the anti-α-synuclein antibody, or antigen-binding fragment thereof, in accordance with the invention. A reduction in α-synuclein spreading may comprise a reduction in the extent of spreading compared to a reference level of spreading. A reference level of α-synuclein spreading may be based on models of disease progression, e.g., on the normal level or average level of α-synuclein spreading in reference subject(s) having the same disease or disorder or the same α-synucleinopathy as the subject, or a reference subject having the same degree of disease progression as the subject being treated. A reference level of α-synuclein spreading may be based on the amount of spreading seen in one or more control subjects over a similar period of time, such as a population of control subjects not administered an anti-α-synuclein antibody, or antigen-binding fragment thereof, in accordance with the invention, such as one or more placebo-treated subjects. A reduction in α-synuclein spreading may be a reduction in the amount of α-synuclein in the areas of spreading compared to the reference level, such as a lower amount of α-synuclein in the areas of spread. A reduction in α-synuclein spreading may be a reduction in the extent of spreading compared to the reference level, such as a reduction in the number of areas to which α-synuclein spreads or a reduction in the area or volume of spread. A reduction in α-synuclein spreading may be a reduction or delay in the progression of α-synuclein spreading compared to a reference level such as a reduction or delay in the spreading of α-synuclein spreading within an area or to different areas.

Free unbound α-synuclein levels in the CSF of a subject may be measured by any suitable means. As used herein, the term “free unbound α-synuclein” refers to α-synuclein that is not bound to an anti-α-synuclein antibody or antigen-binding fragment thereof. Said free unbound α-synuclein can apply to α-synuclein in its monomeric or oligomeric form, or in aggregated form. These terms generally apply to any pathologic form of α-synuclein. Accordingly, the level of free, unbound, α-synuclein in a sample may be assessed by a method that comprises separating α-synuclein that is bound to the antibody or fragment from α-synuclein that is not bound to the antibody or fragment. Free unbound α-synuclein levels in the CSF of a subject may be measured by a method that comprises removing α-synuclein that is bound to the antibody, or antigen-binding fragment thereof, from a sample of CSF, and then assessing the amount of α-synuclein that remains in the sample. For example, CSF samples may be subjected to immunoprecipitation (IP) to remove α-synuclein bound to the antibody, or antigen-binding fragment thereof, such that unbound “free” α-synuclein remains in the supernatant. Free levels of α-synuclein that remain in the CSF sample (e.g., supernatant) may then be determined by any suitable method, such as by ELISA. For example, levels of free α-synuclein in the CSF supernatant may be assessed using a commercial ELISA kit, e.g., a Mesoscale Discovery ELISA kit (See Example 1) or Sensolyte™ Quantitative ELISA kit, human/mouse/rat, AnaSpec, US, AS-55550. As used herein, “sample” may refer to an in vitro or ex vivo sample that has been obtained from a suitable subject. CSF samples may be taken from a subject by lumbar puncture. A change or reduction in free unbound α-synuclein levels in the CSF may be determined by comparing the levels of free, unbound, α-synuclein in CSF samples taken from a subject at different time points (e.g., before treatment in accordance with the invention (e.g., at baseline) and during and/or after treatment) and determining whether there has been any change over that period of time. A CSF sample may be obtained from the subject before the start of a method of the present invention, or before an administration forming part of a method of the invention, and may be compared with one or more CSF samples obtained from the subject during and/or after a method of the invention, such as CSF samples taken after one or more administrations of an antibody, or antigen binding fragment thereof, in accordance with the invention. A reduction in free, unbound α-synuclein in the CSF may comprise a reduction in the level of α-synuclein in the CSF of the subject during a method of the invention, e.g., that the level is lower after the method of the invention compared to the level in the CSF of the subject before the method of the invention (e.g., at baseline). A reduction in free, unbound α-synuclein in the CSF may be a reduction or delay in the progression of levels of free, unbound α-synuclein levels in the CSF compared to that seen in a control subject that is not administered the anti-α-synuclein antibody, or antigen-binding fragment thereof, in accordance with the invention. A reduction in α-synuclein spreading may comprise a reduction or delay in the progression of levels of free, unbound α-synuclein levels in the CSF compared to a reference level of free, unbound α-synuclein. A reference level of free, unbound α-synuclein may be based on models of disease progression, e.g., on the normal level or average level of free, unbound α-synuclein in the CSF in reference subject(s) having the same disease or disorder or the same α-synucleinopathy as the subject, or a reference subject having the same degree of disease progression as the subject being treated. A reference level of free, unbound α-synuclein may be based on the amount of free, unbound α-synuclein seen in the CSF of one or more control subjects, such as a population of control subjects not administered an anti-α-synuclein antibody, or antigen-binding fragment thereof, in accordance with the invention, such as one or more placebo-treated subjects. One or more CSF samples obtained from the subject during and/or after a method of the invention, such as CSF samples taken after one or more administrations of an antibody or antigen-binding fragment thereof in accordance with the invention may be compared with one or more CSF samples obtained from one or more placebo-treated subjects. The level of free, unbound α-synuclein may be assessed in the CSF samples, or the relative levels of free, unbound α-synuclein may be compared between the CSF samples. The amounts (or relative amounts) of free, unbound α-synuclein may then be compared between the CSF samples taken at different time points in relation to the method of the invention, in order to determine whether the method of the invention, or the administration of one or more doses of the antibody or antigen binding fragment thereof, has reduced the amount of free, unbound α-synuclein in the CSF. A lower level of free, unbound α-synuclein in the CSF during or after a method of the invention, compared to the level of free, unbound α-synuclein in the CSF of the subject before the method of the invention, may indicate that the method of the invention leads to a reduction in free, unbound α-synuclein in the CSF. A lower level of free, unbound α-synuclein in the CSF of the subject during or after a method of the invention compared to the level of free, unbound α-synuclein in the control or placebo treated subject(s) or compared to a reference level of free, unbound α-synuclein, may indicate that the method of the invention leads to a reduction in free, unbound α-synuclein in the CSF. A delayed or reduced increase in the level of free, unbound α-synuclein in the CSF of the subject (based on a comparison between the level before the method of the invention (e.g., at baseline) and the level during or after the method of the invention), when compared to the change in the during or after a method of the invention compared to the level of free, unbound α-synuclein in the level of free, unbound α-synuclein in the CSF over the same period of time in control or placebo treated subject(s) or compared to a reference level of change of free, unbound α-synuclein, indicates that the method of the invention leads to a reduction in free, unbound α-synuclein in the CSF.

Free unbound α-synuclein levels in the brain ISF of a subject may be measured by any suitable means. For example, ISF samples may be taken by microdialysis, for example as described in Herukka et al. (2015) J. Alzheimers Dis. 46(1): 261-9. The level of free, unbound, α-synuclein in an ISF sample may be assessed by a method as discussed above in relation to the measurement of free, unbound α-synuclein levels in the CSF, such as by a method that comprises separating α-synuclein that is bound to the antibody or fragment from α-synuclein that is not bound to the antibody or fragment. For example, ISF samples may be subjected to immunoprecipitation to remove α-synuclein bound to the antibody or antigen-binding fragment thereof. Free levels of α-synuclein in the remaining supernatant may be determined, e.g., by ELISA. A change in the level of free unbound α-synuclein levels in the ISF may be determined by comparing the levels of α-synuclein in ISF samples taken from a subject at different time points, in the same way discussed above in relation to assessing a reduction in CSF samples. A change or reduction in free unbound α-synuclein levels in the ISF may be determined by comparing the levels of free, unbound, α-synuclein in ISF samples taken from a subject at different time points (e.g., before treatment in accordance with the invention (e.g., at baseline) and during and/or after treatment) and determining whether there has been any change over that period of time. An ISF sample obtained from the subject before the start of a method of the present invention, or before an administration forming part of a method of the invention, may be compared with one or more ISF samples obtained from the subject during and/or after a method of the invention, such as ISF samples taken after one or more administrations of an antibody or antigen binding fragment thereof in accordance with the invention. Alternatively, one or more ISF samples obtained from the subject during and/or after a method of the invention, such as ISF samples taken after one or more administrations of an antibody or antigen-binding fragment thereof in accordance with the invention may be compared with one or more ISF samples obtained from a placebo-treated subject. The level of free, unbound α-synuclein may be assessed in the ISF samples, or the relative levels of free, unbound α-synuclein may be compared between the ISF samples. A reduction in free, unbound α-synuclein in the ISF may comprise a reduction in the level of α-synuclein in the ISF of the subject during a method of the invention, e.g., that the level is lower after the method of the invention compared to the level in the ISF of the subject before the method of the invention (e.g., at baseline). A reduction in free, unbound α-synuclein in the ISF may be a reduction or delay in the progression of levels of free, unbound α-synuclein levels in the ISF compared to that seen in a control subject that is not administered the anti-α-synuclein antibody, or antigen-binding fragment thereof, in accordance with the invention. A reduction in α-synuclein spreading may comprise a reduction or delay in the progression of levels of free, unbound α-synuclein levels in the ISF compared to a reference level of free, unbound α-synuclein. A reference level of free, unbound α-synuclein may be based on models of disease progression, e.g., on the normal level or average level of free, unbound α-synuclein in the ISF in reference subject(s) having the same disease or disorder or the same α-synucleinopathy as the subject, or a reference subject having the same degree of disease progression as the subject being treated. A reference level of free, unbound α-synuclein may be based on the amount of free, unbound α-synuclein seen in the ISF of one or more control subjects, such as a population of control subjects not administered an anti-α-synuclein antibody, or antigen-binding fragment thereof, in accordance with the invention, such as one or more placebo-treated subjects. One or more ISF samples obtained from the subject during and/or after a method of the invention, such as ISF samples taken after one or more administrations of an antibody or antigen-binding fragment thereof in accordance with the invention may be compared with one or more ISF samples obtained from one or more placebo-treated subjects. The level of free, unbound α-synuclein may be assessed in the ISF samples, or the relative levels of free, unbound α-synuclein may be compared between the ISF samples. The amounts (or relative amounts) of free, unbound α-synuclein may then be compared between the ISF samples taken at different time points in relation to the method of the invention, in order to determine whether the method of the invention, or the administration of one or more doses of the antibody or antigen binding fragment thereof, has reduced the amount of free, unbound α-synuclein in the ISF. A lower level of free, unbound α-synuclein in the ISF during or after a method of the invention, compared to the level of free, unbound α-synuclein in the ISF of the subject before the method of the invention, may indicate that the method of the invention leads to a reduction in free, unbound α-synuclein in the ISF. A lower level of free, unbound α-synuclein in the ISF of the subject during or after a method of the invention compared to the level of free, unbound α-synuclein in the control or placebo treated subject(s) or compared to a reference level of free, unbound α-synuclein, may indicate that the method of the invention leads to a reduction in free, unbound α-synuclein in the ISF. A delayed or reduced increase in the level of free, unbound α-synuclein in the ISF of the subject (based on a comparison between the level before the method of the invention (e.g., at baseline) and the level during or after the method of the invention), when compared to the change in the during or after a method of the invention compared to the level of free, unbound α-synuclein in the level of free, unbound α-synuclein in the ISF over the same period of time in control or placebo treated subject(s) or compared to a reference level of change of free, unbound α-synuclein, may indicate that the method of the invention leads to a reduction in free, unbound α-synuclein in the ISF.

Treatment with an anti-α-synuclein antibody or antigen-binding fragment thereof in accordance with the present invention may alter (e.g., improve, inhibit, reduce, stabilize, or delay or reduce an increase in) one or more symptoms or characteristics of an α-synucleinopathy in the subject based on scoring or measurement of one or more clinical measures, outcomes or symptoms in the subject, e.g., based on a clinical scoring system. A variety of well-known clinical scoring systems exist for the assessment of subjects having α-synucleinopathies. Treatment with an anti-α-synuclein antibody or antigen-binding fragment thereof in accordance with the present invention may alter (e.g., improve, inhibit, reduce, stabilize, or delay or reduce an increase in) the score of the subject in any such scoring system, such an UMSARS, CGI-S, SCOPA AUT, or MDS-UPDRS score of a subject, as described herein. Preferably reduction in any of the scores or levels described herein is a reduction as compared to the score of a subject not treated with such an anti-α-synuclein antibody, or antigen-binding fragment thereof (e.g., a control-or placebo-treated subject, or group of control-or placebo-treated subjects). Reduction in the scores or levels described herein compared to such a control are therefore indicative of reduction in progression of the α-synucleinopathy. For example, the method of the invention may reduce a score in the subject, e.g., based on a comparison between the score of the subject before the method of the invention (e.g., at baseline) and the score of the subject during or after the method of the invention. The method of the invention may delay or reduce an increase in a score in the subject. This may reflect a delay or reduction in the progression of the α-synucleinopathy in the subject. For example, the score may be stabilized, e.g., the score may not increase, e.g., based on a comparison between the score of the subject before the method of the invention (e.g., at baseline) and the score of the subject during or after the method of the invention. The change in the score (e.g., based on a comparison between the score of the subject before the method of the invention (e.g., at baseline) and the score of the subject during or after the method of the invention) may be less than would be expected based on a control, such as a control subject that is not administered the anti-α-synuclein antibody, or antigen-binding fragment thereof, in accordance with the invention. For example, the change in the score in the subject may be reduced or delayed compared to a reference value representing the expected change in score for such a subject over that period of time. This may reflect a delay or reduction in the progression of the α-synucleinopathy in the subject. A reference value may be based on models of disease progression, e.g., on the normal level or average scores or change in scores seen in reference subject(s) having the same disease or disorder or the same α-synucleinopathy as the subject, or reference subject(s) having the same degree of disease progression as the subject being treated. A reference value may be based on the scores of one or more control subjects, such as a population of control subjects not administered an anti-α-synuclein antibody, or antigen-binding fragment thereof, in accordance with the invention, such as one or more placebo-treated subjects. A reference value may be based on the change in scores of one or more control subjects over the same period of time, such as a population of control subjects not administered an anti-α-synuclein antibody, or antigen-binding fragment thereof, in accordance with the invention, such as one or more placebo-treated subjects.

The score may be the UMSARS score. The score may be a modified version of the UMSARS Part I. The method may reduce progression of an α-synucleinopathy as measured by a modified version of the UMSARS Part I. Accordingly, treatment with the anti-α-synuclein antibody or antigen-binding fragment thereof may reduce the subject's score in a modified version of the UMSARS Part I. Preferably, the method reduces progression of an α-synucleinopathy as measured by a modified version of the UMSARS Part I. The UMSARS Part I may be modified to comprise fewer than 12 questions. The UMSARS Part I may be modified so that answers to the questions are scored from 0-3 or 1-4.

Treatment with the anti-α-synuclein antibody or antigen-binding fragment thereof may reduce the subject's UMSARS total score. The method may reduce progression of an α-synucleinopathy as measured by the UMSARS total score. The UMSARS total score may comprise all items from UMSARS Parts I, II, III and IV. The UMSARS total score may comprise all items from a modified UMSARS Part I and all items from UMSARS Parts II, III and IV.

Treatment with the anti-α-synuclein antibody or antigen-binding fragment thereof may reduce the subject's UMSARS Part I score. The method may reduce progression of an α-synucleinopathy as measured by the UMSARS Part I score.

Treatment with the anti-α-synuclein antibody or antigen-binding fragment thereof may reduce the subject's UMSARS Part II score. The method may reduce progression of an α-synucleinopathy as measured by the UMSARS Part II score.

Treatment with the anti-α-synuclein antibody or antigen-binding fragment thereof may reduce the subject's 11-item UMSARS score. The method may reduce progression of an α-synucleinopathy as measured by the 11-item UMSARS score. The 11-item UMSARS score is described in Palma et al. (2021) Clinical Auton. Res. 31: 157-164 and consists of UMSARS Part I questions 2, 3, 6, 7 and 11 and UMSARS Part II questions 1, 2, 9, 11, 12 and 14. These 11 items demonstrated the highest cumulative standardized effect in an analysis of longitudinal natural history from the Natural History Study of the Synucleinopathies.

Treatment with the anti-α-synuclein antibody or antigen-binding fragment thereof may reduce the subject's Clinical Global Impression-Severity (CGI-S) scale score. The method may reduce progression of an α-synucleinopathy as measured by the CGI-S scale score. The CGI-S (described in more detail in Busner and Targum (2007) Psychiatry (Edgmont) 4(7): 28-37) is used to assess the clinician's impression of the subject's clinical condition. The clinician scores the severity of the subject's illness on a 7-point scale ranging from 1 for normal, not at all ill to 7 for among the most extremely ill patient. The rating is based on observed and reported symptoms, behaviour, and function in the past 7 days and should reflect the average severity level across the 7 days.

Treatment with the anti-α-synuclein antibody or antigen-binding fragment thereof may reduce the subject's score in the Scales for Outcomes in Parkinson's Disease—Autonomic Dysfunction (SCOPA-AUT) test. The method may reduce progression of an α-synucleinopathy as measured by the SCOPA-AUT test. The SCOPA-AUT (described in more detail in Visser et al. (2004) Mov. Disord. 19(11): 1306-12) is used to evaluate autonomic symptoms in patients with Parkinson's disease. The scale is self-completed by patients and consists of 25 items assessing gastrointestinal, urinary, cardiovascular, thermoregulatory, pupillomotor, and sexual symptoms.

Treatment with the anti-α-synuclein antibody or antigen-binding fragment thereof (e.g., at a fixed dose every 4 weeks for at least 1 year)may improve survival compared to placebo.

Treatment with the anti-α-synuclein antibody or antigen-binding fragment thereof may reduce the subject's score in the Movement Disorder Society-Sponsored Revision of the Unified Parkinson's Disease Rating Scale (MDS-UPDRS). The method may reduce progression of an α-synucleinopathy as measured by the MDS-UPDRS. For example, the method may increase time to meaningful progression on motor signs of the disease, as assessed by an at least 5-point increase in MDS-UPDRS Part III score from baseline. The MDS-UPDRS (described in more detail in Goetz et al. (2008) Mov. Disord. 23(15): 21129-2170) is used to evaluate various aspects of Parkinson's disease including non-motor and motor experiences of daily living and motor complications.

Treatment with the anti-α-synuclein antibody or antigen-binding fragment thereof may improve the subject's cognition. For example, treatment with the anti-α-synuclein antibody or antigen-binding fragment thereof may increase the subject's score in the MoCA. The method may reduce progression of an α-synucleinopathy as measured by the subject's score in the MoCA.

Treatment with the anti-α-synuclein antibody or antigen-binding fragment thereof (e.g., at a fixed dose every 4 weeks for at least 1 year) may improve gait, balance and/or Parkinsonian signs compared to a control or reference value, e.g., compared to progression in placebo-treated subject(s). The method may reduce progression of an α-synucleinopathy as measured by gait, balance and/or Parkinsonian signs.

Treatment with the anti-α-synuclein antibody or antigen-binding fragment thereof (e.g. at a fixed dose every 4 weeks for at least 1 year) may improve blood pressure and/or heart rate, for example as measured using the orthostatic hypotension questionnaire (OHQ). The method may reduce progression of an α-synucleinopathy as measured using the OHQ. The OHQ (described in more detail in Kaufmann et al. (2011) Clin. Auton. Res. 22(2): 79-90) is used to assess burden of symptoms associated with orthostatic hypotension and comprises two components: a six-item symptom assessment scale; and a four-item daily activity scale.

Treatment with the anti-α-synuclein antibody or antigen-binding fragment thereof (e.g. at a fixed dose every 4 weeks for at least 1 year) may improve one or more symptoms of REM behavior disorder, for example as measured using polysomnography with or without video; Innsbruck REM sleep behavior disorder questionnaire (described in more detail in Frauscher et al. (2012) Mov. Disord. 27(13): 1673-1678); REM behavior disorder questionnaire Hong Kong (described in more detail in Shen et al. (2014) Sleep Med. 15(8): 952-958); CGI (e.g. CGI-C); PGI (e.g. PGI-I or PGI-C; described in more detail in Guy (1976) ECDEU assessment manual for psychopharmacology, U.S. Department of Health, Education, and Welfare); and/or CIRUS-RBD questionnaire (described in more detail in Gilat et al. (2020) Mov. Disord. 35(2): 344-349. The method may reduce progression of one or more symptoms of REM behavior disorder, for example as measured using polysomnography with or without video; Innsbruck REM sleep behavior questionnaire; REM behavior disorder questionnaire Hong Kong; CGI-C; PGI-I/C; and/or CIRUS-RBD questionnaire.

Treatment with the anti-α-synuclein antibody or antigen-binding fragment thereof (e.g., at a fixed dose every 4 weeks for at least 1 year) may reduce plasma neurofilament light chain levels, reduce total α-synuclein levels, reduces free unbound CSF α-synuclein levels, and/or reduce CSF neurofilament light chain levels. The method may reduce progression of an α-synucleinopathy as measured by plasma neurofilament light chain levels, total α-synuclein levels, free unbound CSF α-synuclein levels, and/or CSF neurofilament light chain levels.

Reduction in any of the scores or levels described herein may be a reduction compared to a control measurement in the subject (e.g., the baseline score in the subject immediately before treatment was initiated). For example, treatment with the anti-α-synuclein antibody or antigen-binding fragment thereof may reduce a score or level by at least 10%, 20%, 30%, 40%, 50%, 60%, 70%, 80%, 90% or 95% compared to baseline.

Preferably, reduction in any of the scores or levels described herein may be as compared to the score or level in a subject or group of subjects not administered an anti-α-synuclein antibody or antigen-binding fragment thereof (e.g., a control subject, a placebo-treated subject, a group of control subjects or a group of placebo-treated subjects). For example, treatment with the anti-α-synuclein antibody or antigen-binding fragment thereof may provide a reduced score or level by at least 10%, 20%, 30%, 40%, 50%, 60%, 70%, 80%, 90% or 95% compared to the corresponding score or level in a control subject, a placebo-treated subject, a group of control subjects, or a group of placebo treated subjects. Preferably the comparison is with a group of placebo treated subjects, such as a typical or average change in score obtained from a group of placebo-treated subjects. Preferably the control or placebo-treated subject(s) are subjects having the same disease or disorder as the subject being treated. Preferably the control or placebo treated subject(s) have the same or similar level of disease progression as the subject being treated at the start of treatment. Preferably the control or placebo treated subjects(s) have the same scope or a similar score to the subject being treated at the start of treatment.

Preferably, the anti-α-synuclein antibody or antigen-binding fragment thereof is administered to the subject, e.g., at a fixed dose every 4 weeks for at least 1 year, and the method reduces free unbound α-synuclein levels in the cerebrospinal fluid (CSF) of the subject. The anti-α-synuclein antibody or antigen-binding fragment thereof may be administered to the subject at a fixed dose, e.g., every 4 weeks for at least 1 year, and the method may reduce free unbound α-synuclein levels in the CSF of the subject by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce free unbound α-synuclein levels in the CSF of the subject from baseline by at least 10%, at least 20%, at least 30%, at least 40%, at least 50%, at least 60%, at least 70%, at least 80%, at least 90% or at least 95%. Preferably, the anti-α-synuclein antibody or antigen-binding fragment thereof may be administered to the subject at a fixed dose, e.g., every 4 weeks for at least 1 year, and the method may reduce free unbound α-synuclein levels in the CSF of the subject from baseline by at least 30%. The anti-α-synuclein antibody or antigen-binding fragment thereof may be administered to the subject at a fixed dose, e.g., every 4 weeks for at least 1 year, and the method may reduce free unbound α-synuclein levels in the CSF of the subject by at least 10%, at least 20%, at least 30%, at least 40%, at least 50%, at least 60%, at least 70%, at least 80%, at least 90% or at least 95% compared to a control or reference value, e.g., compared to the level in placebo-treated subject(s). Preferably, the anti-α-synuclein antibody or antigen-binding fragment thereof is administered to the subject at a fixed dose, e.g., every 4 weeks for at least 1 year, and the method reduces free unbound α-synuclein levels in the CSF of the subject by at least 30% compared to a control or reference value, e.g., compared to the level in placebo-treated subject(s).

The anti-α-synuclein antibody or antigen-binding fragment thereof may be administered to the subject at a fixed dose, e.g., every 4 weeks for at least 1 year, and the method may reduce free unbound α-synuclein levels in the interstitial fluid (ISF) of the subject. The anti-α-synuclein antibody or antigen-binding fragment thereof may be administered to the subject at a fixed dose, e.g., every 4 weeks for at least 1 year, and the method may reduce free unbound α-synuclein levels in the ISF of the subject by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce free unbound α-synuclein levels in the ISF of the subject from baseline by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce free unbound α-synuclein levels in the ISF of the subject from baseline by at least 30%. The anti-α-synuclein antibody or antigen-binding fragment thereof may be administered to the subject at a fixed dose, e.g., every 4 weeks for at least 1 year, and the method may reduce free unbound α-synuclein levels in the ISF of the subject by at least 10%, at least 20%, at least 30%, at least 40%, at least 50%, at least 60%, at least 70%, at least 80%, at least 90% or at least 95% compared to a control or reference value, e.g., compared to the level in placebo-treated subject(s). The anti-α-synuclein antibody or antigen-binding fragment thereof may be administered to the subject at a fixed dose, e.g., every 4 weeks for at least 1 year, and the method may reduce free unbound α-synuclein levels in the ISF of the subject by at least 30% compared to a control or reference value, e.g., compared to the level in placebo-treated subject(s).

The anti-α-synuclein antibody or antigen-binding fragment thereof may be administered to the subject at a fixed dose, e.g., every 4 weeks for at least 1 year, and the method may reduce α-synuclein spreading in the subject. The anti-α-synuclein antibody or antigen-binding fragment thereof may be administered to the subject at a fixed dose, e.g., every 4 weeks for at least 1 year, and the method may reduce α-synuclein spreading in the subject by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce α-synuclein spreading in the subject from baseline by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce α-synuclein spreading in the subject from baseline by at least 30%. The anti-α-synuclein antibody or antigen-binding fragment thereof may be administered to the subject at a fixed dose, e.g., every 4 weeks for at least 1 year, and the method may reduce α-synuclein spreading in the subject by at least 10%, at least 20%, at least 30%, at least 40%, at least 50%, at least 60%, at least 70%, at least 80%, at least 90% or at least 95% compared to a control or reference value, e.g., compared to α-synuclein spreading in placebo-treated subject(s). The anti-α-synuclein antibody or antigen-binding fragment thereof may be administered to the subject at a fixed dose, e.g., every 4 weeks for at least 1 year, and the method may reduce α-synuclein spreading in the subject by at least 30% compared to a control or reference value, e.g., compared to α-synuclein spreading in placebo-treated subject(s).

The anti-α-synuclein antibody or antigen-binding fragment thereof may be administered to the subject at a fixed dose, e.g., every 4 weeks for at least 1 year, and the method may reduce the subject's score in a modified version of the UMSARS Part I. The anti-α-synuclein antibody or antigen-binding fragment thereof may be administered to the subject at a fixed dose, e.g., every 4 weeks for at least 1 year, and the method may reduce the subject's score in a modified version of the UMSARS Part I by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce the subject's score in a modified version of the UMSARS Part I from baseline by at least 10%, at least 20%, at least 30%, at least 40%, at least 50%, at least 60%, at least 70%, at least 80%, at least 90% or at least 95%. The methods or dosage regimens disclosed herein may reduce the subject's modified UMSARS Part I score from baseline by at least 30%. The anti-α-synuclein antibody or antigen-binding fragment thereof may be administered to a subject at a fixed dose, e.g., every 4 weeks for at least 1 year, and the method may reduce the subject's score in a modified version of the UMSARS Part I by at least 10%, at least 20%, at least 30%, at least 40%, at least 50%, at least 60%, at least 70%, at least 80%, at least 90% or at least 95% compared to a control or reference value, e.g., compared to the score in placebo-treated subject(s). The anti-α-synuclein antibody or antigen-binding fragment thereof may be administered to the subject at a fixed dose, e.g., every 4 weeks for at least 1 year, and the method may reduce the subject's score in a by at least 30% compared to a control or reference value, e.g., compared to the score in placebo-treated subject(s).

The anti-α-synuclein antibody or antigen-binding fragment thereof may be administered to the subject at a fixed dose, e.g., every 4 weeks for at least 1 year, and the method may reduce the subject's UMSARS total score. The anti-α-synuclein antibody or antigen-binding fragment thereof may be administered to the subject at a fixed dose, e.g., every 4 weeks for at least 1 year, and the method may reduce the subject's UMSARS total score by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce the subject's UMSARS total score from baseline by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce the subject's UMSARS total score from baseline by at least 30%. The anti-α-synuclein antibody or antigen-binding fragment thereof may be administered to the subject at a fixed dose, e.g., every 4 weeks for at least 1 year, and the method may reduce the subject's UMSARS total score by at least 10%, at least 20%, at least 30%, at least 40%, at least 50%, at least 60%, at least 70%, at least 80%, at least 90% or at least 95% compared to a control or reference value, e.g., compared to the score in placebo-treated subject(s). The anti-α-synuclein antibody or antigen-binding fragment thereof may be administered to the subject at a fixed dose, e.g., every 4 weeks for at least 1 year, and the method may reduce the subject's UMSARS total score by at least 30% compared to a control or reference value, e.g., compared to the score in placebo-treated subject(s).

The anti-α-synuclein antibody or antigen-binding fragment thereof may be administered to the subject at a fixed dose, e.g., every 4 weeks for at least 1 year, and the method may reduce the subject's UMSARS Part I score. The anti-α-synuclein antibody or antigen-binding fragment thereof may be administered to the subject at a fixed dose, e.g., every 4 weeks for at least 1 year, and the method may reduce the subject's UMSARS Part I score by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce the subject's UMSARS Part I score from baseline by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce the subject's UMSARS Part I score from baseline by at least 30%. The anti-α-synuclein antibody or antigen-binding fragment thereof may be administered to the subject at a fixed dose, e.g., every 4 weeks for at least 1 year, and the method may reduce the subject's UMSARS Part I score by at least 10%, at least 20%, at least 30%, at least 40%, at least 50%, at least 60%, at least 70%, at least 80%, at least 90% or at least 95% compared to a control or reference value, e.g., compared to the score in placebo-treated subject(s). The anti-α-synuclein antibody or antigen-binding fragment thereof may be administered to the subject at a fixed dose, e.g., every 4 weeks for at least 1 year, and the method may reduce the subject's UMSARS Part I score by at least 30% compared to a control or reference value, e.g., compared to the score in placebo-treated subject(s).

The anti-α-synuclein antibody or antigen-binding fragment thereof may be administered to the subject at a fixed dose, e.g., every 4 weeks for at least 1 year, and the method may reduce the subject's UMSARS Part II score. The anti-α-synuclein antibody or antigen-binding fragment thereof may be administered to the subject at a fixed dose, e.g., every 4 weeks for at least 1 year, and the method may reduce the subject's UMSARS Part II score by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce the subject's UMSARS Part II score from baseline by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce the subject's UMSARS Part II score from baseline by at least 30%. The anti-α-synuclein antibody or antigen-binding fragment thereof may be administered to the subject at a fixed dose, e.g., every 4 weeks for at least 1 year, and the method may reduce the subject's UMSARS Part II score by at least 10%, at least 20%, at least 30%, at least 40%, at least 50%, at least 60%, at least 70%, at least 80%, at least 90% or at least 95% compared to a control or reference value, e.g., compared to the score in placebo-treated subject(s). The anti-α-synuclein antibody or antigen-binding fragment thereof may be administered to the subject at a fixed dose, e.g., every 4 weeks for at least 1 year, and the method may reduce the subject's UMSARS Part II score by at least 30% compared to a control or reference value, e.g., compared to the score in placebo-treated subject(s).

The anti-α-synuclein antibody or antigen-binding fragment thereof may be administered to the subject at a fixed dose, e.g., every 4 weeks for at least 1 year, and the method may reduce the subject's 11-item UMSARS score. The anti-α-synuclein antibody or antigen-binding fragment thereof may be administered to the subject at a fixed dose, e.g., every 4 weeks for at least 1 year, and the method may reduce the subject's 11-item UMSARS score by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce the subject's 11-item UMSARS score from baseline by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce the subject's 11-item UMSARS score from baseline by at least 10%, at least 20%, at least 30%. The anti-α-synuclein antibody or antigen-binding fragment thereof may be administered to the subject at a fixed dose, e.g., every 4 weeks for at least 1 year, and the method may reduce the subject's 11-item UMSARS score by at least 10%, at least 20%, at least 30%, at least 40%, at least 50%, at least 60%, at least 70%, at least 80%, at least 90% or at least 95% compared to a control or reference value, e.g., compared to the score in placebo-treated subject(s). The anti-α-synuclein antibody or antigen-binding fragment thereof may be administered to the subject at a fixed dose, e.g., every 4 weeks for at least 1 year, and the method may reduce the subject's 11-item UMSARS score by at least 30% compared to a control or reference value, e.g., compared to the score in placebo-treated subject(s).

The anti-α-synuclein antibody or antigen-binding fragment thereof may be administered to the subject at a fixed dose, e.g., every 4 weeks for at least 1 year, and the method may reduce the subject's CGI-S score. The anti-α-synuclein antibody or antigen-binding fragment thereof may be administered to the subject at a fixed dose, e.g., every 4 weeks for at least 1 year, and the method may reduce the subject's CGI-S score by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce the subject's CGI-S score from baseline by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce the subject's CGI-S score from baseline by at least 30%. The anti-α-synuclein antibody or antigen-binding fragment thereof may be administered to the subject at a fixed dose, e.g., every 4 weeks for at least 1 year, and the method may reduce the subject's CGI-S score by at least 10%, at least 20%, at least 30%, at least 40%, at least 50%, at least 60%, at least 70%, at least 80%, at least 90% or at least 95% compared to a control or reference value, e.g., compared to the score in placebo-treated subject(s). The anti-α-synuclein antibody or antigen-binding fragment thereof may be administered to the subject at a fixed dose, e.g., every 4 weeks for at least 1 year, and the method may reduce the subject's CGI-S score by at least 30% compared to a control or reference value, e.g., compared to the score in placebo-treated subject(s).

The anti-α-synuclein antibody or antigen-binding fragment thereof may be administered to the subject at a fixed dose, e.g., every 4 weeks for at least 1 year, and the method may reduce the subject's SCOPA-AUT score. The anti-α-synuclein antibody or antigen-binding fragment thereof may be administered to the subject at a fixed dose, e.g., every 4 weeks for at least 1 year, and the method may reduce the subject's SCOPA-AUT score by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce the subject's SCOPA-AUT score from baseline by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce the subject's SCOPA-AUT score from baseline by at least 30%. The anti-α-synuclein antibody or antigen-binding fragment thereof may be administered to the subject at a fixed dose, e.g., every 4 weeks for at least 1 year, and the method may reduce the subject's SCOPA-AUT score by at least 10%, at least 20%, at least 30%, at least 40%, at least 50%, at least 60%, at least 70%, at least 80%, at least 90% or at least 95% compared to a control or reference value, e.g., compared to the score in placebo-treated subject(s). The anti-α-synuclein antibody or antigen-binding fragment thereof may be administered to the subject at a fixed dose, e.g., every 4 weeks for at least 1 year, and the method may reduce the subject's SCOPA-AUT score by at least 30% compared to a control or reference value, e.g., compared to the score in placebo-treated subject(s).

Treatment with an anti-α-synuclein antibody or antigen-binding fragment thereof may improve the subject's cognitive, motor and/or speech function. Accordingly, the anti-α-synuclein antibody or antigen-binding fragment thereof may be administered to the subject at a fixed dose, e.g., every 4 weeks for at least 1 year, and the method may improve the subject's cognitive, motor and/or speech function. The method may reduce progression of an α-synucleinopathy as measured by the subject's cognitive, motor and/or speech function.

Treatment with an anti-α-synuclein antibody or antigen-binding fragment thereof, may improve autonomic function such as orthostatic blood pressure and/or orthostatic heart rate. Accordingly, the anti-α-synuclein antibody or antigen-binding fragment thereof may be administered to the subject at a fixed dose, e.g., every 4 weeks for at least 1 year, and the method may improve the subject's orthostatic blood pressure and/or orthostatic heart rate. The method may reduce progression of an α-synucleinopathy as measured by orthostatic blood pressure and/or orthostatic heart rate.

The anti-α-synuclein antibody or antigen-binding fragment thereof may be administered to the subject at a fixed dose, e.g., every 4 weeks for at least 1 year, and the method may reduce the subject's MDS-UPDRS score. The anti-α-synuclein antibody or antigen-binding fragment thereof may be administered to the subject at a fixed dose, e.g., every 4 weeks for at least 1 year, and the method may reduce the subject's MDS-UPDRS score by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce the subject's MDS-UPDRS score from baseline by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may reduce the subject's MDS-UPDRS score from baseline by at least 30%. The anti-α-synuclein antibody or antigen-binding fragment thereof may be administered to the subject at a fixed dose, e.g., every 4 weeks for at least 1 year, and the method may reduce the subject's MDS-UPDRS score by at least 10%, at least 20%, at least 30%, at least 40%, at least 50%, at least 60%, at least 70%, at least 80%, at least 90% or at least 95% compared to a control or reference value, e.g., compared to the score in placebo-treated subject(s). The anti-α-synuclein antibody or antigen-binding fragment thereof may be administered to the subject at a fixed dose, e.g., every 4 weeks for at least 1 year, and the method may reduce the subject's MDS-UPDRS score by at least 30% compared to a control or reference value, e.g., compared to the score in placebo-treated subject(s).

The anti-α-synuclein antibody or antigen-binding fragment thereof may be administered to the subject at a fixed dose, e.g., every 4 weeks for at least 1 year, and the method may increase the subject's MoCA score. The anti-α-synuclein antibody or antigen-binding fragment thereof may be administered to the subject at a fixed dose, e.g., every 4 weeks for at least 1 year, and the method may increase the subject's MoCA score by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may increase the subject's MoCA score from baseline by at least 10%, at least 20%, at least 30%, at least 40%, at least 50%, at least 60%, at least 70%, at least 80%, at least 90% or at least 95%. The anti-α-synuclein antibody or antigen-binding fragment thereof may be administered to the subject at a fixed dose, e.g., every 4 weeks for at least 1 year, and the method may increase the subject's MoCA score from baseline by at least 30%. The anti-α-synuclein antibody or antigen-binding fragment thereof may be administered to the subject at a fixed dose, e.g., every 4 weeks for at least 1 year, and the method may increase the subject's MoCA score by at least 10%, at least 20%, at least 30%, at least 40%, at least 50%, at least 60%, at least 70%, at least 80%, at least 90% or at least 95% compared to a control or reference value, e.g., compared to the score in placebo-treated subject(s). The anti-α-synuclein antibody or antigen-binding fragment thereof may be administered to the subject at a fixed dose, e.g., every 4 weeks for at least 1 year, and the method may increase the subject's MoCA score by at least 30% compared to a control or reference value, e.g., compared to the score in placebo-treated subject(s).

The prevention or treatment defined herein or in accordance with the invention may be applied as a sole therapy or may involve, in addition to the anti-α-synuclein antibody or antigen-binding fragment, administration of other agents or other therapies. The anti-α-synuclein antibody or antigen-binding fragment may be administered in combination with another agent or therapy for the treatment or prevention of an α-synucleinopathy. The anti-α-synuclein antibody or antigen-binding fragment may be administered in combination with an established agent or therapy normally used in the treatment of α-synucleinopathies, such as L-3,4-dihydroxyphenylalanine (L-DOPA), dopamine (receptor) agonists, catechol-O-methyltransferase (COMT) inhibitors, and/or monoamine oxidase type B (MAO-B) inhibitors. The administration of other agents or therapies may be in combination with, or as an adjunct to, or in conjunction with, the antibody or antigen-binding fragment used in the invention and may be by way of simultaneous, sequential or separate dosing of the individual components of the treatment. The antibody or antigen-binding fragment may be provided in the same composition as an additional therapeutic agent. The antibody or antigen-binding fragment may be provided in a different composition to any other therapeutic agents being administered as part of the same treatment. The anti-α-synuclein antibody or antigen-binding fragment may be administered in combination with one or more other anti-α-synuclein antibodies or antigen-binding fragments thereof, such as one or more other anti-α-synuclein antibodies or antigen-binding fragments thereof as described herein. Each anti-α-synuclein antibody as defined herein may be administered at a fixed dose of 50-5,000 mg, or a fixed dose as defined herein. Where more than one anti-α-synuclein antibody as defined herein is administered in a method of the invention, each administration may be at a fixed dose of 50-5,000 mg, or a fixed dose as defined herein.

The combination treatment may be carried out in any way as deemed necessary or convenient by the person skilled in the art and for the purpose of this specification, no limitations with regard to the order, amount, repetition or relative amount of the compounds to be used in combination is contemplated.

### 4.2 Anti-α-synuclein Antibodies or Antigen-Binding Fragments Thereof

The present invention uses an anti-α-synuclein antibody or antigen-binding fragment thereof. For example, in methods of the invention an anti-α-synuclein antibody or antigen-binding fragment thereof is administered to treat or prevent an α-synucleinopathy. Similarly, the invention provides an anti-α-synuclein antibody or antigen-binding fragment thereof for use in a method of treating or preventing an α-synucleinopathy. Kits and compositions of the invention comprise an anti-α-synuclein antibody or antigen-binding fragment thereof. In any aspect of the invention, the anti-α-synuclein antibody or antigen-binding fragment thereof may be an anti-α-synuclein antibody or antigen-binding fragment thereof as described below.

As used herein, the term “anti-α-synuclein antibody” means an antibody that binds to α-synuclein. The anti-α-synuclein antibody, or antigen-binding fragment thereof, may bind specifically to α-synuclein, for example it may bind to α-synuclein and not other related molecules. For example, the anti-α-synuclein antibody, or antigen-binding fragment thereof, may bind to α-synuclein but not β-synuclein or γ-synuclein. The anti-α-synuclein antibody, or antigen-binding fragment thereof, may bind to human α-synuclein but not human β-synuclein or human γ-synuclein. The anti-α-synuclein antibody, or antigen-binding fragment thereof, may bind to human, rat and/or cynomolgus α-synuclein. The anti-α-synuclein antibody, or antigen-binding fragment thereof, may bind to human, rat and cynomolgus α-synuclein.

### 4.2.1 Amino Acid Sequences of aslo0452 ngl-3 and Antigen-Binding Fragments Thereof

The anti-α-synuclein antibody or antigen-binding fragment thereof may be the antibody aslo0452 ngl-3, or an antigen-binding fragment thereof. Aslo0452 ngl-3 is a human α-synuclein monoclonal antibody (mAb) derived from parental clone asyn0087 and is directed against the C-terminus of human α-synuclein. Aslo0452 ngl-3 includes a variable heavy chain region of SEQ ID NO: 14 and a variable light chain region of SEQ ID NO: 19. Aslo0452 ngl-3 includes a light chain having the amino acid sequence of SEQ ID NO: 17. As a result of lysine clipping during the manufacturing process, the final lysine of the aslo0452 ngl-3 heavy chain may be absent. Accordingly, aslo0452 ngl-3 may include a heavy chain having the amino acid sequence of SEQ ID NO: 12 or SEQ ID NO: 62. Preferably, aslo0452 ngl-3 includes a heavy chain having the amino acid sequence of SEQ ID NO: 12. Aslo0452 ngl-3 may include a heavy chain having the amino acid sequence of SEQ ID NO: 12 and a light chain having the amino acid sequence of SEQ ID NO: 17. Aslo0452 ngl-3 may include a heavy chain having the amino acid sequence of SEQ ID NO: 62 and a light chain having the amino acid sequence of SEQ ID NO: 17. Aslo0452 ngl-3 binds to both monomeric and aggregated forms of human α-synuclein. Aslo0452 ngl-3 has been shown to reduce α-synuclein spreading in vivo. In addition, aslo0452 ngl-3 has been shown to bind to reduce free unbound α-synuclein levels in the interstitial fluid (ISF) and cerebrospinal fluid (CSF) in animal models of disease. Aslo0452 ngl-3 and its production is described in more detail in WO2017/207739, which is incorporated by reference herein in its entirety. As shown herein, aslo0452 ngl-3 is the first anti-α-synuclein antibody shown to reduce free unbound levels of α-synuclein in the CSF of human subjects. This allows measurement of aslo0452 ngl-3 target engagement during treatment.

The anti-α-synuclein antibody or antigen-binding fragment thereof for use in the invention may have the six CDRs of antibody aslo0452 ngl-3. Thus, preferably, the anti-α-synuclein antibody or antigen-binding fragment thereof comprises:
a) three heavy chain CDRs having sequences:
   (i) H-CDR1 of SEQ ID NO: 5,
   (ii) H-CDR2 of SEQ ID NO: 15; and
   (iii) H-CDR3 of SEQ ID NO: 16, and
b) three light chain CDRs having sequences:
   (i) L-CDR1 of SEQ ID NO: 20,
   (ii) L-CDR2 of SEQ ID NO: 10, and
   (iii) L-CDR3 of SEQ ID NO: 21.

As used herewith, “H-CDR” refers to a complementary determining region (CDR) on the heavy chain region, and “L-CDR” refers to a complementary determining region (CDR) on the light chain region of an antibody or antigen-binding fragment thereof.

The anti-α-synuclein antibody or antigen-binding fragment thereof may comprise a variable heavy chain region encoded by a nucleotide sequence having at least 80%, 85%, 90%, 91%, 92%, 93%, 94%, 95%, 96%, 97%, 98%, 99%, or 100% identity to the nucleotide sequence defined by SEQ ID NO: 13, and/or a variable light chain region encoded by a nucleotide sequence having at least 80%, 85%, 90%, 91%, 92%, 93%, 94%, 95%, 96%, 97%, 98%, 99%, or 100% identity to the nucleotide sequence defined by SEQ ID NO: 18.

The anti-α-synuclein antibody or antigen-binding fragment thereof may comprise a variable heavy chain region encoded by a nucleotide sequence defined by SEQ ID NO: 13 and/or a variable light chain region encoded by a nucleotide sequence defined by SEQ ID NO: 18. Preferably, the anti-α-synuclein antibody or antigen-binding fragment thereof comprises a variable heavy chain region encoded by a nucleotide sequence defined by SEQ ID NO: 13 and a variable light chain region encoded by a nucleotide sequence defined by SEQ ID NO: 18.

The anti-α-synuclein antibody or antigen-binding fragment thereof may comprise a variable heavy chain region having the amino acid sequence of SEQ ID NO: 14, or having at least 80%, 85%, 90%, 91%, 92%, 93%, 94%95%, 96%, 97%, 98%, 99%, or 100% identity to the amino acid sequence defined by SEQ ID NO: 14, and/or a variable light chain region having the amino acid sequence defined of SEQ ID NO: 19 or having at least 80%, 85%, 90%, 91%, 92%, 93%, 94%, 95%, 96%, 97%, 98%, 99%, or 100% identity to the amino acid sequence defined by SEQ ID NO: 19.

The anti-α-synuclein antibody or antigen-binding fragment thereof may comprise a variable heavy chain region having the amino acid sequence of SEQ ID NO: 14 and/or a variable light chain region having the amino acid sequence of SEQ ID NO: 19. Preferably, the anti-α-synuclein antibody or antigen-binding fragment thereof comprises a variable heavy chain region having the amino acid sequence of SEQ ID NO: 14 and a variable light chain region having the amino acid sequence of SEQ ID NO: 19. The anti-α-synuclein antibody or antigen-binding fragment thereof may comprise a variable heavy chain region consisting of an amino acid sequence defined by SEQ ID NO: 14 and a variable light chain region consisting of an amino acid sequence defined by SEQ ID NO: 19.

Preferably, the anti-α-synuclein antibody or antigen-binding fragment thereof comprises a variable heavy chain region having at least 80%, 85%, 90%, 91%, 92%, 93%, 94%, 95%, 96%, 97%, 98%, 99%, or 100% identity to the sequence defined by SEQ ID NO: 14 and a variable light chain region having at least 80%, 85%, 90%, 91%, 92%, 93%, 94%, 95%, 96%, 97%, 98%, 99%, or 100% identity to the sequence defined by SEQ ID NO: 19 and may further comprise:
a) three heavy chain CDRs having sequences:
   (i) H-CDR1 of SEQ ID NO: 5,
   (ii) H-CDR2 of SEQ ID NO: 15; and
   (iii) H-CDR3 of SEQ ID NO: 16, and
b) three light chain CDRs having sequences:
   (i) L-CDR1 of SEQ ID NO: 20,
   (ii) L-CDR2 of SEQ ID NO: 10; and
   (iii) L-CDR3 of SEQ ID NO: 21.

The anti-α-synuclein antibody or antigen-binding fragment thereof may comprise a heavy chain having an amino acid sequence defined by SEQ ID NO: 12 or SEQ ID NO: 62. Preferably, the anti-α-synuclein antibody or antigen-binding fragment thereof comprises a heavy chain having an amino acid sequence defined by SEQ ID NO: 12. The anti-α-synuclein antibody or antigen-binding fragment thereof may comprise a light chain having an amino acid sequence defined by SEQ ID NO: 17. The anti-α-synuclein antibody or antigen-binding fragment thereof may comprise a heavy chain having an amino acid sequence defined by SEQ ID NO: 12 or SEQ ID NO: 62 and/or a light chain having an amino acid sequence defined by SEQ ID NO: 17. Preferably, the anti-α-synuclein antibody or antigen-binding fragment thereof comprises a heavy chain having an amino acid sequence defined by SEQ ID NO: 12 or SEQ ID NO: 62 and a light chain having an amino acid sequence defined by SEQ ID NO: 17. Preferably, the anti-α-synuclein antibody or antigen-binding fragment thereof comprises a heavy chain having an amino acid sequence defined by SEQ ID NO: 12 and a light chain having an amino acid sequence defined by SEQ ID NO: 17. The anti-α-synuclein antibody or antigen-binding fragment thereof may comprise a heavy chain consisting of an amino acid sequence defined by SEQ ID NO: 12 or SEQ ID NO: 62 and a light chain consisting of an amino acid sequence defined by SEQ ID NO: 17. The anti-α-synuclein antibody or antigen-binding fragment thereof may comprise a heavy chain consisting of an amino acid sequence defined by SEQ ID NO: 12 and a light chain consisting of an amino acid sequence defined by SEQ ID NO: 17.

### 4.2.2 Amino Acid Sequences of Aslo0543 and Antigen-Binding Fragments Thereof

The anti-α-synuclein antibody or antigen-binding fragment thereof may be the antibody aslo0543, or an antigen-binding fragment thereof. Aslo0543 is a human α-synuclein monoclonal antibody (mAb) derived from parental clone asyn0087 and is directed against the C-terminus of human α-synuclein. Aslo0543 includes a variable heavy chain region of SEQ ID NO: 24 and a variable light chain region of SEQ ID NO: 30. Aslo0543 includes a heavy chain (SEQ ID NO: 22) and a light chain (SEQ ID NO: 28). Aslo0543 binds to both monomeric and aggregated forms of human α-synuclein. Aslo0543 and its production is described in more detail in WO2017/207739, which is incorporated by reference herein in its entirety.

The anti-α-synuclein antibody or antigen-binding fragment thereof may have the six CDRs of antibody aslo0543. Thus, the anti-α-synuclein antibody or antigen-binding fragment thereof may comprise:
a) three heavy chain CDRs having sequences:
   (i) H-CDR1 of SEQ ID NO: 25,
   (ii) H-CDR2 of SEQ ID NO: 26; and
   (iii) H-CDR3 of SEQ ID NO: 27, and
b) three light chain CDRs having sequences:
   (i) L-CDR1 of SEQ ID NO: 31,
   (ii) L-CDR2 of SEQ ID NO: 32; and
   (iii) L-CDR3 of SEQ ID NO: 33.

The anti-α-synuclein antibody or antigen-binding fragment thereof may comprise a variable heavy chain region encoded by a nucleotide sequence having at least 80%, 85%, 90%, 91%, 92%, 93%, 94%, 95%, 96%, 97%, 98%, 99%, or 100% identity to the nucleotide sequence defined by SEQ ID NO: 23, and/or a variable light chain region encoded by a nucleotide sequence having at least 80%, 85%, 90%, 91%, 92%, 93%, 94%, 95%, 96%, 97%, 98%, 99%, or 100% identity to the nucleotide sequence defined by SEQ ID NO: 29.

The anti-α-synuclein antibody or antigen-binding fragment thereof may comprise a variable heavy chain region encoded by a nucleotide sequence defined by SEQ ID NO: 23 and/or a variable light chain region encoded by a nucleotide sequence defined by SEQ ID NO: 29. Preferably, the anti-α-synuclein antibody or antigen-binding fragment thereof comprises a variable heavy chain region encoded by a nucleotide sequence defined by SEQ ID NO: 23 and a variable light chain region encoded by a nucleotide sequence defined by SEQ ID NO:

The anti-α-synuclein antibody or antigen-binding fragment thereof may comprise a variable heavy chain region having the amino acid sequence of SEQ ID NO: 24, or having at least 80%, 85%, 90%, 91%, 92%, 93%, 94%, 95%, 96%, 97%, 98%, 99%, or 100% identity to the amino acid sequence defined by SEQ ID NO: 24, and/or a variable light chain region having the amino acid sequence defined of SEQ ID NO: 30 or having at least 80%, 85%, 90%, 91%, 92%, 93%, 94%, 95%, 96%, 97%, 98%, 99%, or 100% identity to the amino acid sequence defined by SEQ ID NO: 30.

The anti-α-synuclein antibody or antigen-binding fragment thereof may comprise a variable heavy chain region having the amino acid sequence of SEQ ID NO: 24 and/or a variable light chain region having the amino acid sequence of SEQ ID NO: 30. Preferably, the anti-α-synuclein antibody or antigen-binding fragment thereof comprises a variable heavy chain region having the amino acid sequence of SEQ ID NO: 24 and a variable light chain region having the amino acid sequence of SEQ ID NO: 30. The anti-α-synuclein antibody or antigen-binding fragment thereof may comprise a variable heavy chain region consisting of an amino acid sequence defined by SEQ ID NO: 24 and a variable light chain region consisting of an amino acid sequence defined by SEQ ID NO: 30.

The anti-α-synuclein antibody or antigen-binding fragment thereof may comprise a variable heavy chain region having at least 80%, 85%, 90%, 91%, 92%, 93%, 94%, 95%, 96%, 97%, 98%, 99% or 100% identity to the sequence defined by SEQ ID NO: 24 and a variable light chain region having at least 80%, 85%, 90%, 91%, 92%, 93%, 94%, 95%, 96%, 97%, 98%, 99% or 100% identity to the sequence defined by SEQ ID NO: 30 and may further comprise:
a) three heavy chain CDRs having sequences:
   (i) H-CDR1 of SEQ ID NO: 25,
   (ii) H-CDR2 of SEQ ID NO: 26; and
   (iii) H-CDR3 of SEQ ID NO: 27, and
b) three light chain CDRs having sequences:
   (i) L-CDR1 of SEQ ID NO: 31,
   (ii) L-CDR2 of SEQ ID NO: 32; and
   (iii) L-CDR3 of SEQ ID NO: 33.

The anti-α-synuclein antibody or antigen-binding fragment thereof may comprise a variable heavy chain region having at least 80%, 85%, 90% or 95% identity to the sequence defined by SEQ ID NO: 14 and a variable light chain region having at least 80%, 85%, 90%, or 92% identity to the sequence defined by SEQ ID NO: 19 and may further comprise:
a) three heavy chain CDRs having sequences:
   (i) H-CDR1 of SEQ ID NO: 25,
   (ii) H-CDR2 of SEQ ID NO: 26; and
   (iii) H-CDR3 of SEQ ID NO: 27, and
b) three light chain CDRs having sequences:
   (i) L-CDR1 of SEQ ID NO: 31,
   (ii) L-CDR2 of SEQ ID NO: 32; and
   (iii) L-CDR3 of SEQ ID NO: 33.

The anti-α-synuclein antibody or antigen-binding fragment thereof may comprise a variable heavy chain region having at least 80%, 85%, 90%, 91%, 92%, 93%, 94%, 95%, 96%, 97%, 98%, 99%, or 100% identity to the sequence defined by SEQ ID NO: 24 and a variable light chain region having at least 80%, 85%, 90%, or 92% identity to the sequence defined by SEQ ID NO: 30 and may further comprise:
a) three heavy chain CDRs having sequences:
   (i) H-CDR1 of SEQ ID NO: 5,
   (ii) H-CDR2 of SEQ ID NO: 15; and
   (iii) H-CDR3 of SEQ ID NO: 16, and
b) three light chain CDRs having sequences:
   (i) L-CDR1 of SEQ ID NO: 20,
   (ii) L-CDR2 of SEQ ID NO: 10; and
   (iii) L-CDR3 of SEQ ID NO: 21.

The anti-α-synuclein antibody or antigen-binding fragment thereof may comprise a heavy chain having an amino acid sequence defined by SEQ ID NO: 22 and/or a light chain having an amino acid sequence defined by SEQ ID NO: 28. The anti-α-synuclein antibody or antigen-binding fragment thereof may comprise a heavy chain having an amino acid sequence defined by SEQ ID NO: 22 and a light chain having an amino acid sequence defined by SEQ ID NO: 28. The anti-α-synuclein antibody or antigen-binding fragment thereof may comprise a heavy chain consisting of an amino acid sequence defined by SEQ ID NO: 22 and a light chain consisting of an amino acid sequence defined by SEQ ID NO: 28.

### 4.2.3 Functional Properties of Antibodies and Antigen-Binding Fragments Thereof for Use in the Invention

Any of the anti-α-synuclein antibodies or antigen-binding fragments thereof disclosed herein, such as any anti-α-synuclein antibodies or antigen-binding fragments thereof for use in the invention, may have any one or more of the functional properties of aslo0452 ngl-3, e.g., any of the aslo0452 ngl-3 functional properties recited herein. Any of the anti-α-synuclein antibodies or antigen-binding fragments thereof disclosed herein, such as any anti-α-synuclein antibodies or antigen-binding fragments thereof for use in the invention, may have any one or more of the functional properties of aslo0543, e.g., any of the aslo0543 functional properties recited herein.

Like the parental clone asyn0087, the aslo0452 ngl-3 and aslo0543 antibodies bind the C-terminal region (residues 96-140) of human α-synuclein. More specifically the aslo0452 ngl-3 and aslo0543 antibodies bind the region comprised between about amino acid 102 and about amino acid 130 of human α-synuclein (e.g., SEQ ID NO: 1). Accordingly, the present invention may use an anti-α-synuclein antibody or antigen-binding fragment thereof that specifically binds the C-terminal region of human α-synuclein. The anti-α-synuclein antibody or antigen-binding fragment thereof may specifically bind the region comprising about amino acid 102 to about amino acid 130 of human α-synuclein (e.g., SEQ ID NO: 1), for example it may specifically bind to the stretch of amino acids 102 to 130 of human α-synuclein, or it may specifically bind one or more of amino acids 102 to 130 in human α-synuclein. The anti-α-synuclein antibody or antigen-binding fragment thereof may specifically bind to a region comprising about amino acid 120 to about amino acid 130 of human α-synuclein (SEQ ID NO: 1). The anti-α-synuclein antibody or antigen-binding fragment thereof may bind 1, 2, 3, 4, 5, 6, 7, 8, 9, 10, 11, 12, 13, 14, 15, 16, 17, 18, 19, 20, 21, 22, 23, 24, 25, 26, 27, 28 or 29 amino acids in the region comprising about amino acid 102 to about amino acid 130 of human α-synuclein (SEQ ID NO: 1), such as 1, 2, 3, 4, 5, 6, 7, 8, 9, 10, 11, 12, 13, 14, 15, 16, 17, 18, 19, 20, 21, 22, 23, 24, 25, 26, 27, 28 or 29 of the amino acids 102 to 130 of human α-synuclein (SEQ ID NO: 1). The epitope of the anti-α-synuclein antibody or antigen-binding fragment thereof may comprise amino acids 102-130 of human α-synuclein (SEQ ID NO: 1), or may comprise one or more amino acids from the region comprising amino acids 102-130 of human α-synuclein. For example, the epitope of the anti-α-synuclein antibody or antigen-binding fragment thereof may comprise 1, 2, 3, 4, 5, 6, 7, 8, 9, 10, 11, 12, 13, 14, 15, 16, 17, 18, 19, 20, 21, 22, 23, 24, 25, 26, 27, 28 or 29 amino acids from amino acids 102 to 130 of human α-synuclein (SEQ ID NO: 1). The anti-α-synuclein antibody or antigen-binding fragment thereof binds to an epitope that is not the same as the antibody bound by the 9E4 antibody.

The aslo0452 ngl-3 and aslo0543 antibodies are selective for α-synuclein. Accordingly, preferably, the present invention may use an anti-α-synuclein antibody or antigen-binding fragment thereof that binds human α-synuclein but does not bind human β-synuclein or human γ-synuclein. The specificity of an anti-α-synuclein antibody or antigen-binding fragment thereof may be determined using any suitable means, e.g., a HTRF epitope competition assay that measures the binding of biotinylated human α-synuclein to the antibody or antigen-binding fragment thereof in solution. In the HTRF assay, related synucleins (e.g., α-synuclein, β-synuclein and γ-synuclein) may be titrated into the assay and the selectivity of the antibody or antigen-binding fragment thereof can be measured by assessing the degree of inhibition of biotinylated human α-synuclein binding to the antibody or antigen-binding fragment thereof. IC₅₀ values may be determined by curve fitting the data to a four parameter logistic equation using PRISM 6 software (Graphpad).

The aslo0452 ngl-3 and aslo0543 antibodies bind to each of human, rat and cynomolgus α-synuclein. The ability of aslo0452 ngl-3 and aslo0543 to bind to human, cynomolgus monkey and rat α-synuclein is indicative of binding to a different epitope on human alpha-synuclein as compared to antibodies that do not bind to human, cynomolgus monkey and rat α-synuclein. The aslo0452 ngl-3 and aslo0543 antibodies are thus capable of being used for in vivo safety evaluation and investigation in cynomolgus monkey and rat models of disease. Accordingly, preferably, the present invention may use an anti-α-synuclein antibody or antigen-binding fragment thereof that binds to human, rat and/or cynomolgus α-synuclein. Preferably, the anti-α-synuclein antibody or antigen-binding fragment thereof binds to human, rat and cynomolgus α-synuclein. Binding to human, rat or cynomolgus α-synuclein may be determined using any suitable means, e.g., a HTRF epitope competition assay. The HTRF epitope assay may measure the binding of biotinylated human α-synuclein to the antibody or antigen-binding fragment thereof. Human, cynomolgus monkey and rat α-synuclein may be titrated into the assay, and the selectivity of the antibody or antigen-binding fragment thereof can be assessed by measuring the degree of inhibition of biotinylated human α-synuclein binding to the antibody or antigen-binding fragment thereof. IC50 values are determined, e.g., by curve fitting the data to a four parameter logistic equation using PRISM 6 software (Graphpad). Species cross reactivity of the antibody or antigen-binding fragment thereof may be confirmed using a direct binding HTRF assay format. The antibody or antigen-binding fragment thereof is titrated into the assay to compete for human or cynomolgus monkey or rat α-synuclein binding to the antibody or antigen-binding fragment thereof by HTRF assay.

The aslo0452 ngl-3 and aslo0543 antibodies bind to human α-synuclein with high affinity. The aslo0452 ngl-3 and aslo0543 antibodies have been reported in WO 2017/207739 to bind to alpha-synuclein with a K_{D} of 106 PM (95% CI 10-292 pM) and 113 PM (95% CI 5-333 pM) respectively as measured using Octet analysis. Accordingly, the present invention may use an anti-α-synuclein antibody or antigen-binding fragment thereof that binds to human α-synuclein with high affinity. The anti-α-synuclein antibody or antigen-binding fragment thereof may bind to alpha-synuclein with a K_{D} of less than 500 picoMolar (pM), less than 400 pM, less than 300 pM, less than 200 pM, less than 150 pM, less than 120 pM, less than 110 pM or 106 PM or less as measured for example using Octet analysis. The affinity of an antibody or antigen-binding fragment thereof may be assessed using an avi-tag α-synuclein-Flag-His tagged molecule. The antibody or antigen-binding fragment may be pre-mixed with varying concentrations of the ligand until equilibrium is reached. The amount of free antibody or antigen-binding fragment thereof may then be measured using the Octet by capturing free antibody or antigen-binding fragment thereof using biotinylated α-synuclein immobilized onto streptavidin coated sensors. The amount of free antibody or antigen-binding fragment thereof detected at each α-synuclein concentration may then be plotted against the concentration of ligand and software used to calculate the equilibrium dissociation constant (K_{D}). The anti-α-synuclein antibody or antigen-binding fragment thereof may bind to monomeric alpha-synuclein with a K_{D} of less than 500 picoMolar (pM), less than 400 pM, less than 300 pM, less than 200 pM, less than 150 pM, less than 120 pM, less than 110 pM or 106 pM or less as measured for example using Octet analysis. Preferably, the anti-α-synuclein antibody or antigen-binding fragment thereof binds to monomeric alpha-synuclein with a K_{D} of less than 500 pM as measured for example using Octet analysis. Preferably, the anti-α-synuclein antibody or antigen-binding fragment thereof binds to monomeric human alpha-synuclein with a K_{D} of less than 500 picoMolar (pM), less than 400 pM, less than 300 pM, less than 200 pM, less than 150 PM, less than 120 pM, less than 110 pM or 106 PM or less as measured for example using Octet analysis. Particularly preferably, the anti-α-synuclein antibody or antigen-binding fragment thereof binds to monomeric human alpha-synuclein with a K_{D} of less than 500 pM as measured for example using Octet analysis.

The aslo0452 ngl-3 and aslo0543 antibodies have been reported in WO 2017/207739 to bind to alpha-synuclein with a K_{D} of 74 PM (95% CI 15-177 PM) and 108 PM (95% CI 34-223 pM) respectively as measured using KinExA analysis. The aslo0452 ngl-3 Fab fragment was reported in WO2017/207739 to bind to alpha-synuclein with a K_{D} of 174 PM (95% CI 15-177 pM) as measured using KinExA analysis. Accordingly, the anti-α-synuclein antibody or antigen-binding fragment thereof used in the invention may bind to alpha-synuclein with a KD of less than 400 picoMolar (pM), less than 300 pM, less than 250 pM, less than 200 pM, less than 150 pM, less than 120 pM, less than 110 pM, less than 100 pM, less than 80 pM or 74 pM or less as measured for example using KinExA analysis. The solution phase affinity (K_{D}) of an antibody or antigen-binding fragment thereof may be assessed using monomeric human biotinylated α-synuclein using a KinExA instrument (Sapidyne Instruments). The antibody or antigen-binding fragment thereof may be pre-mixed with varying concentrations of each ligand until equilibrium is reached. The amount of free antibody may then be measured using the KinExA by capturing free antibody or antigen-binding fragment thereof using α-synuclein coated boards, washing away unbound material and detecting bound antibody using a fluorescently labelled species specific antibody. The amount of free antibody detected at each α-synuclein concentration may then be plotted against the concentration of ligand and the KinExA software may be used to calculate the equilibrium dissociation constant (K_{D}). The anti-α-synuclein antibody or antigen-binding fragment thereof may bind to monomeric alpha-synuclein with a K_{D} of less than 400 picoMolar (pM), less than 300 pM, less than 250 pM, less than 200 pM, less than 150 pM, less than 120 PM, less than 110 pM, less than 100 pM, less than 80 pM or 74 PM or less as measured for example using KinExA analysis. Preferably, the anti-α-synuclein antibody or antigen-binding fragment thereof binds to monomeric alpha-synuclein with a K_{D} of less than 400 pM as measured for example using KinExA analysis. The anti-α-synuclein antibody or antigen-binding fragment thereof may bind to monomeric human alpha-synuclein with a K_{D} of less than 400 picoMolar (pM), less than 300 pM, less than 250 pM, less than 200 pM, less than 150 pM, less than 120 pM, less than 110 pM, less than 100 pM, less than 80 pM or 74 pM or less as measured for example using KinExA analysis. Particularly preferably, the anti-α-synuclein antibody or antigen-binding fragment thereof binds to monomeric human alpha-synuclein with a K_{D} of less than 400 pM as measured for example using KinExA analysis.

The aslo0452 ngl-3 and aslo0543 antibodies bind to native endogenous human α-synuclein. As used herein “native endogenous α-synuclein” means the α-synuclein present in tissues of the subject to be treated. Accordingly, the present invention may use an anti-α-synuclein antibody or antigen-binding fragment thereof that binds to native endogenous human α-synuclein. Binding of an antibody or antigen-binding fragment thereof to native endogenous human α-synuclein may be determined using any suitable means, e.g., by flow cytometry using α-synuclein positive and negative cell lines. For example, α-synuclein positive cells (e.g., SHSY5Y neuroblastoma cells) and α-synuclein negative cells (e.g., BT-20 breast cancer cells) may be fixed in formaldehyde and permeabilized prior to incubation with an anti-α-synuclein antibody or antigen-binding fragment thereof, positive control or isotype control antibodies. Bound antibody may then be detected by incubation with an appropriate secondary antibody. The cells may then be analysed with a FACS machine, e.g., FACS Canto II apparatus (Becton Dickinson, Franklin Lakes, NJ) and data analysis may be performed using appropriate software, e.g., FlowJo Software (Tree Star, Ashland, OR). Binding to native endogenous human α-synuclein may be confirmed by a shift in fluorescence signal in the presence of the antibody or antigen-binding fragment thereof in the α-synuclein positive cells compared to isotype control and/or secondary antibody alone and no shift in the presence of the antibody or antigen-binding fragment thereof in the α-synuclein negative cells compared to the isotype control and/or secondary antibody alone. Preferably, the anti-α-synuclein antibody or antigen-binding fragment thereof binds to native endogenous human α-synuclein in vivo. The ability of an anti-α-synuclein antibody or antigen-binding fragment thereof to bind to native endogenous human α-synuclein in vivo may be determined by administering the antibody or antigen-binding fragment thereof to a subject followed by determination of binding of the antibody or antigen-binding fragment thereof to native endogenous α-synuclein in a sample obtained from that subject. The anti-α-synuclein antibody or antigen-binding fragment thereof may bind to native endogenous human α-synuclein in the brain and/or CSF of a subject to be treated.

The aslo0452 ngl-3 and aslo0543 antibodies bind to aggregates of human α-synuclein. Accordingly, the anti-α-synuclein antibody or antigen-binding fragment thereof used in the invention may bind to aggregates of human α-synuclein, such as oligomers and/or polymers and/or aggregates of human α-synuclein. The anti-α-synuclein antibody or antigen-binding fragment thereof used in the invention may bind to aggregates of human α-synuclein such as Lewy bodies or Lewy body like aggregates. Binding of an anti-α-synuclein antibody or antigen-binding fragment thereof to aggregates of human α-synuclein may be determined using a DELFIA® antibody capture assay. For example, a mouse IgG1 version of the antibody or antigen-binding fragment thereof may be immobilized onto the well of a 96-well microtiter plate (Nunc). After blocking, aggregated or monomeric human α-synuclein may be incubated in the wells. Following washing, captured human α-synuclein may be detected by addition of the human IgG1 TM version of the same antibody or antigen-binding fragment thereof and subsequently an anti-human IgG-Europium conjugate (Perkin Elmer) or anti-human IgG-HRP conjugate. After incubation and washing, a detection substrate (e.g., TMB or DELFIA Enhancer solution) may be added. The plate may then be read on a microtiter plate reader. This assay is not capable of detecting monomeric α-synuclein.

The aslo0452 ngl-3 and aslo0543 antibodies bind to monomeric human α-synuclein. The anti-α-synuclein antibody or antigen-binding fragment thereof used in the invention may bind to monomeric human α-synuclein. The anti-α-synuclein antibody or antigen-binding fragment thereof used in the invention may bind to monomeric and/or oligomeric human α-synuclein. Binding of an anti-α-synuclein antibody or antigen-binding fragment thereof to monomeric forms of human α-synuclein may be determined using any suitable means, e.g., a KinExA assay. The anti-α-synuclein antibody or antigen-binding fragment thereof may bind monomeric and/or oligomeric α-synuclein in the CSF of the subject. The anti-α-synuclein antibody or antigen-binding fragment thereof may bind monomeric α-synuclein in the CSF of the subject. The anti-α-synuclein antibody or antigen-binding fragment thereof may bind oligomeric α-synuclein in the CSF of the subject. Binding of an antibody or antigen-binding fragment thereof to monomeric α-synuclein in the CSF of a subject may be determined using any suitable means, for example, using an ELISA which specifically detects the presence of monomeric α-synuclein to measure binding of the antibody or antigen-binding fragment thereof to monomeric α-synuclein from the CSF of a subject. Binding of an antibody or antigen-binding fragment thereof to oligomeric α-synuclein in the CSF of a subject may be determined using any suitable means, for example, using an ELISA which specifically detects the presence of oligomeric α-synuclein to measure binding of the antibody or antigen-binding fragment thereof to oligomeric α-synuclein from the CSF of a subject. The anti-α-synuclein antibody or antigen-binding fragment thereof may bind monomeric and/or oligomeric α-synuclein in the CSF or ISF of the subject in vivo. The anti-α-synuclein antibody or antigen-binding fragment thereof may bind monomeric α-synuclein in the CSF of the subject in vivo.

The aslo0452 ngl-3 and aslo0543 antibodies are capable of binding and sequestering both monomeric and aggregated forms of alpha-synuclein. The anti-α-synuclein antibody or antigen-binding fragment thereof used in the invention may sequester α-synuclein. The term “sequester” means to bind a molecule in such a way that the molecule is prevented or inhibited from forming aggregates, or prevented or inhibited from forming further aggregates. An antibody or binding fragment thereof that sequesters α-synuclein may bind to α-synuclein outside a cell, such as in the CSF or ISF, and prevent it from entering cells. An antibody or binding fragment thereof that sequesters α-synuclein may bind to monomeric α-synuclein and prevent it from forming aggregates. An antibody or binding fragment thereof that sequesters α-synuclein may bind to α-synuclein and prevent it from forming aggregates or prevent it from forming Lewy bodies.

The anti-α-synuclein antibody or antigen-binding fragment thereof used in the invention may bind both monomeric and aggregated forms of α-synuclein. The anti-α-synuclein antibody or antigen-binding fragment thereof may sequester both monomeric and aggregated forms of a-synuclein. The anti-α-synuclein antibody or antigen-binding fragment thereof may bind both monomeric and aggregated forms of α-synuclein in vivo. The anti-α-synuclein antibody or antigen-binding fragment thereof may sequester both monomeric and aggregated forms of α-synuclein in vivo. The ability of an antibody or antigen-binding fragment thereof to bind monomeric and aggregated forms of α-synuclein may be determined using any suitable means, e.g., using any of the methods described herein, such as by using a KinExA assay and a DELFIA® antibody capture assay.

The aslo0452 ngl-3 and aslo0543 antibodies bind disease relevant, pathological forms of α-synuclein, e.g., Lewy bodies, Lewy neurites, Lewy dots in Parkinson's disease brain tissues. Accordingly, preferably, the anti-α-synuclein antibody or antigen-binding fragment thereof binds disease relevant, pathological forms of α-synuclein. This may be assessed by immunohistochemical staining using the antibody or antigen-binding fragment thereof in diseased tissue. For example, immunohistochemical staining of Parkinson's disease brain tissue may show the presence of Lewy bodies, Lewy neurites, neuronal aggregates, Lewy dots and background brain tissue. Minimal staining is observed in normal (non-diseased) brain or in tissues not containing α-synuclein. Binding to brain tissue may be assessed in vitro, or in vivo. Preferably, the anti-α-synuclein antibody or antigen-binding fragment thereof binds disease relevant, pathological forms of α-synuclein in vivo, e.g., in a suitable animal model of the α-synucleinopathy.

The aslo0452 ngl-3 antibody reduces α-synuclein levels in the brain interstitial fluid (ISF). In particular, the aslo0452 ngl-3 antibody reduces free unbound α-synuclein levels in the brain interstitial fluid, e.g., in an animal model of an α-synucleinopathy. The anti-α-synuclein antibody or antigen-binding fragment thereof may reduce free unbound α-synuclein levels in the brain interstitial fluid. Reduction of free unbound α-synuclein levels in brain interstitial fluid may be assessed using any suitable means, e.g., as described herein above. Accordingly, the anti-α-synuclein antibody or antigen-binding fragment thereof may reduce α-synuclein levels in the brain interstitial fluid. The anti-α-synuclein antibody or antigen-binding fragment thereof may reduce free unbound α-synuclein levels in the brain interstitial fluid in an animal, e.g., a rat. The anti-α-synuclein antibody or antigen-binding fragment thereof may reduce free unbound α-synuclein levels in the brain interstitial fluid (e.g., of a rat) 1 hour after administration (e.g., intravenous administration). Free unbound α-synuclein levels may be reduced for at least 2 hours, 3 hours, 4 hours, 5 hours, 6 hours, 7 hours, 8 hours, 9 hours, or preferably 10 hours after administration.

The aslo0452 ngl-3 antibody reduces α-synuclein levels in the cerebrospinal fluid (CSF). In particular, the aslo0452 ngl-3 antibody reduces free unbound α-synuclein levels in the cerebrospinal fluid. Accordingly, the anti-α-synuclein antibody or antigen-binding fragment thereof may reduce α-synuclein levels in the cerebrospinal fluid. The anti-α-synuclein antibody or antigen-binding fragment thereof may reduce free, unbound α-synuclein levels in the cerebrospinal fluid. Reduction of free unbound α-synuclein levels in cerebrospinal fluid may be assessed using any suitable means, e.g., as described herein above. The anti-α-synuclein antibody or antigen-binding fragment thereof may reduce free unbound α-synuclein levels in the CSF in an animal, e.g., a rat. The anti-α-synuclein antibody or antigen-binding fragment thereof may reduce free unbound α-synuclein levels in the CSF (e.g., of a rat) 6 hours after administration (e.g., intravenous administration). Free unbound α-synuclein levels may be reduced for at least 12 hours, 18 hours, 24 hours, 30 hours, 36 hours, 42 hours, 48 hours, 54 hours, 60 hours, 66 hours, or preferably 72 hours.

The aslo0452 ngl-3 antibody reduces α-synuclein spreading in vivo. This effect of inhibiting alpha-synuclein spreading is indicative of binding to a different epitope on human alpha-synuclein as compared to antibodies that do not inhibit spreading. Accordingly, preferably, the anti-α-synuclein antibody or antigen-binding fragment thereof reduces α-synuclein spreading in vivo. Reduction of α-synuclein spreading in vivo may be determined using any suitable means, e.g., as described herein above. For example, α-synuclein overexpressing transgenic mice (a-syn tg) are injected with a lentiviral vector that expresses α-synuclein (LV-a-syn) into the right hippocampus and then immunized with the anti-α-synuclein antibody or antigen-binding fragment thereof or control. At the end of the immunization period, the mice are euthanized and their brains fixed. The fixed brains are sectioned and analysed by immunocytochemistry for levels of α-synuclein immunoreactivity ipsilateral and contralateral to the site of the LV-a-syn injection. Reduction in α-synuclein spreading is observed by comparing the levels of α-synuclein spread from the ipsilateral side to the contralateral side of the hippocampus in the anti-α-synuclein antibody or antigen-binding fragment thereof treated mice with the control mice.

### 4.2.4 Additional Anti-α-Synuclein Antibodies or Antigen-Binding Fragments Thereof

The anti-α-synuclein antibody or antigen-binding fragment thereof may compete with antibody aslo0452 ngl-3 and/or antibody aslo0543 for binding to human α-synuclein. The anti-α-synuclein antibody or antigen-binding fragment thereof may bind to the same epitope on human α-synuclein or an overlapping epitope on human α-synuclein as antibody aslo0452 ngl-3 and/or antibody aslo0543. The anti-α-synuclein antibody or antigen-binding fragment thereof may compete with antibody aslo0452 ngl-3 for binding to human α-synuclein. Preferably, the anti-α-synuclein antibody or antigen-binding fragment thereof binds to the same epitope on human α-synuclein or an overlapping epitope on human α-synuclein as antibody aslo0452 ngl-3.

It can readily be determined whether an antibody or antigen-binding fragment thereof binds to the same or overlapping epitope as a reference antibody or antigen-binding fragment. Such methods are a matter of routine in the art. For example, an antibody can be compared to another by biochemical competition assay whereby, two antibodies (one labelled for detection purposes and one not) are incubated simultaneously with a given antigen. If a binding signal is achieved for the labelled antibody then the two antibodies are said to recognize distinct, non-overlapping epitopes on the protein of interest. If no binding signal is obtained then, conversely, they would be characterized as having overlapping epitopes on the protein sequence because binding of one antibody sterically hinders binding of the second antibody. Further, the amino acid location of a given epitope can also be identified using modified proteins such as truncates, linear peptide sequences derived from an antigen's primary amino sequence, species orthologues, and by proteolytic digest and mass spec analysis of an antibody bound to a given protein. These methodologies serve to generate a region of interaction between antibody and antigen.

Additional routine experimentation (such as peptide mutation and binding analyses) can be carried out to confirm whether any observed lack of binding is in fact due to binding the epitope of the invention or if some other phenomenon (such as steric hindrance) is responsible. Such experiments can be carried out using ELISA, RIA, Biacore, flow cytometry or other known antibody binding assays.

For example, for fine mapping of a specific epitope, mathematical models of the epitope:paratope interface can be derived from data generated through solving the structure of the antigen:antibody complex using a high resolution imaging method such as co-crystallization with X-ray diffraction. To confirm the relevance of the mathematical model derived, in terms of identifying key contact residues defining the epitope, point mutagenesis of the antigen must be performed subsequently and an analysis of the effect on strength of binding between antigen and antibody caused by such mutations established. Using this combination of methods an exact map of key contact residues comprising the epitope can be established.

Antibodies or antigen-binding fragments thereof of interest, such as antibodies or antigen binding fragments thereof which bind to the same epitope as a reference antibody or antigen-binding fragment thereof, may be generated by producing variants of that reference antibody or antigen-binding fragment thereof. Accordingly, the antibody or antigen-binding fragment thereof used in the present invention may be a variant of a known anti-α-synuclein antibody or antigen binding fragment thereof, such as a variant of aslo0452 ngl-3 or an antigen-binding fragment thereof, a variant of aslo0543 or an antigen binding fragment thereof, or a variant of asyn0087 or an antigen binding fragment thereof. Thus, in relation to variant antibodies or antigen binding fragments thereof as disclosed herein, the reference antibody may be aslo0452 ngl-3 or an antigen-binding fragment thereof, aslo0543 or an antigen binding fragment thereof, or asyn0087 or an antigen binding fragment thereof

Such variant antibodies or antigen-binding fragments thereof may retain one or more CDRs of the reference antibody or antigen-binding fragment thereof, or may have CDRs sharing a high level of identity with the CDRs of the reference antibody or antigen-binding fragment thereof. For example, the antibody or antigen-binding fragment for use in the present invention may have one or more CDRs that differ by 1 or 2 amino acid residues (e.g., 1 or 2 conservative amino acid substitutions) as compared to any one or more of the specific CDR sequences referred to herein (e.g., the CDRs of aslo0452 ngl-3, aslo0543 or asyn0087, e.g., any one or more of the CDRs having SEQ ID NOs: 5, 15, 16, 20, 10 and 21, any one or more of the CDRs having SEQ ID NOs 25, 26, 27, 31, 32 and 33. The antibody or antigen-binding fragment for use in the present invention may have a set of CDRs that differs by 1 or 2 amino acid residues (e.g., 1 or 2 conservative amino acid substitutions) as compared to any set of specific CDR sequences referred to herein (e.g., the set of CDRs of aslo0452 ngl-3, aslo0543 or asyn0087, e.g., the set of CDRs having SEQ ID NOs: 5, 15, 16, 20, 10 and 21, or the set of CDRs having SEQ ID NOs 25, 26, 27, 31, 32 and 33). The antibody or antigen-binding fragment for use in the present invention may have a set of CDRs comprising one or more of the CDRs as set out in SEQ ID NOs 5, 6, 7, 9, 10, and 11. The antibody or antigen-binding fragment for use in the present invention may have a set of CDRs as set out in SEQ ID NOs 5, 6, 7, 9, 10, and 11.

The anti-α-synuclein antibody or antigen-binding fragment thereof may have one or more variations (e.g., one or more conservative amino acid substitutions) in the CDR amino acid sequences that maintain at least 80%, at least 85%, at least 90%, at least 92%, at least 93%, at least 94%, at least 95%, at least 96%, at least 97%, at least 98% and up to 99% sequence identity to any one or more of the CDRs of antibody aslo0452 ngl-3 (e.g., any one or more of the CDRs having SEQ ID NOs: 5, 15, 16, 20, 10 and 21). The anti-α-synuclein antibody or antigen-binding fragment thereof may have one or more variations in the CDR amino acid sequences that maintain at least 80%, at least 85%, at least 90%, at least 92%, at least 93%, at least 94%, at least 95%, at least 96%, at least 97%, at least 98% and up to 99% sequence identity to any one or more of the CDRs of antibody aslo0452 ngl-3 (e.g., any one or more of the CDRs having SEQ ID NOs: 5, 15, 16, 20, 10 and 21) and retain binding to α-synuclein.

The anti-α-synuclein antibody or antigen-binding fragment thereof may have at least one CDR of the reference antibody. The anti-α-synuclein antibody or antigen-binding fragment thereof may have at least one CDR selected from the CDRs of antibody aslo0452 ngl-3, i.e., at least one CDR selected from any one of SEQ ID NO: 5, SEQ ID NO: 15, SEQ ID NO: 16, SEQ ID NO: 20, SEQ ID NO: 10, and SEQ ID NO: 21.

The anti-α-synuclein antibody or antigen-binding fragment thereof may have at least one, at least two, at least three, at least four, at least five or all six of the CDRs of the reference antibody. The anti-α-synuclein antibody or antigen-binding fragment thereof may have at least one, at least two, at least three, at least four, at least five or all six of the CDRs selected from the CDRs of antibody aslo0452 ngl-3, i.e., SEQ ID NO: 5, SEQ ID NO: 15, SEQ ID NO: 16, SEQ ID NO: 20, SEQ ID NO: 10, and SEQ ID NO: 21.

The anti-α-synuclein antibody or antigen-binding fragment thereof may have all six CDRs of the reference antibody. The anti-α-synuclein antibody or antigen-binding fragment thereof may have all six CDRs of antibody aslo0452 ngl-3. The anti-α-synuclein antibody or antigen-binding fragment thereof may have a set of CDRs in which CDRH1 has the amino acid sequences of SEQ ID NO: 5, CDRH2 has the amino acid sequence of SEQ ID NO: 15, CDRH3 has the amino acid sequence of SEQ ID NO: 16, CDRL1 has the amino acid sequence of SEQ ID NO: 20, CDRL2 has the amino acid sequence of SEQ ID NO: 10, and CDRL3 has the amino acid sequence of SEQ ID NO: 21.

The CDR3 of the heavy chain of the anti-α-synuclein antibody or antigen-binding fragment thereof may be CDR3 of the heavy chain of the reference antibody, and/or the CDR3 of the light chain of the anti-α-synuclein antibody or antigen-binding fragment thereof may be CDR3 of the light chain of the reference antibody. The CDR3 of the heavy chain of the anti-α-synuclein antibody or antigen-binding fragment thereof may be CDR3 of the heavy chain of antibody aslo0452 ngl-3; and/or the CDR3 of the light chain of the anti-α-synuclein antibody or antigen-binding fragment thereof may be CDR3 of the light chain of antibody aslo0452 ngl-3. Thus, the CDR3 of the heavy chain of the anti-α-synuclein antibody or antigen-binding fragment thereof may be CDR3 of SEQ ID NO: 16 of the heavy chain of antibody aslo0452 ngl-3; and/or the CDR3 of the light chain of the anti-α-synuclein antibody or antigen-binding fragment thereof may be CDR3 of SEQ ID NO: 21 of the light chain of antibody aslo0452 ngl-3. The CDR3 of the heavy chain of the anti-α-synuclein antibody or antigen-binding fragment thereof may be CDR3 of the heavy chain of antibody aslo0452 ngl-3. The CDR3 of the light chain of the anti-α-synuclein antibody or antigen-binding fragment thereof may be CDR3 of the light chain of antibody aslo0452 ngl-3.

Variant antibodies or antigen-binding fragments thereof may have one or more variations (e.g., a conservative amino acid substitution) outside the CDRs, such as in one or more of the framework regions.

Variant antibodies or antigen-binding fragments thereof for use in the invention may have a variable heavy chain region (VH) region and/or a variable light chain region (VL) region that shares a high level of identity with the VH and VL regions of the reference antibody or antigen-binding fragment thereof. For example, the antibody or antigen-binding fragment for use in the present invention may have a VL and/or VL that differs by 1, 2, 3, 4, 5, 6, 7, 8, 9, 10, 11, 12 13, 14, 15 or more amino acid residues (e.g., conservative amino acid substitutions) as compared to the VH and/or VL of a reference antibody or antigen binding fragment thereof (e.g., the VH and/or VL of aslo0452 ngl-3, aslo0543 or asyn0087, e.g., the VH and/or VL of SEQ ID NOs 14 and 19 respectively or the VH and/or VL of SEQ ID NOs 24 and 30 respectively or the VH and/or VL of SEQ ID NOs 2 and 3 respectively). The anti-α-synuclein antibody or antigen-binding fragment thereof may comprise a VH and a VL wherein one or both of the VH and the VL has at least 80%, at least 85%, at least 90%, at least 92%, at least 93%, at least 94%, at least 95%, at least 96%, at least 97%, at least 98% and up to 99% sequence identity to the corresponding VH and/or VL of the reference antibody, (e.g., the VH and/or VL of aslo0452 ngl-3, aslo0543 or asyn0087, e.g., the VH and/or VL of SEQ ID NOS 14 and 19 respectively or the VH and/or VL of SEQ ID NOs 24 and 30 respectively or the VH and/or VL of SEQ ID NOs 2 and 3 respectively).

The differences from the reference VH and/or VL sequences may lie outside the CDRs, i.e., the variant may comprise the CDRs from the VH and/or VL of the reference antibody and the differences from the reference VH and/or VL sequences may lie in the framework sequences of the variable domain. The differences from the relevant reference sequence may lie at any position in the VH and/or VL subsequences, for example the variant antibodies or antigen-binding fragments thereof may comprise variations in the CDR sequences, as discussed above.

Variant antibodies or antigen-binding fragments thereof may be used in the present invention. Such variant antibodies or antigen-binding fragments may retain one or more functional properties of the reference antibody from which they are derived, such as any one or more functional properties or characteristics of antibody aslo0542 ngl-3 as described herein. The variant antibody or antigen-binding fragment may specifically bind α-synuclein, e.g., as defined herein. The variant antibody or antigen-binding fragment may specifically bind human α-synuclein, e.g., as defined herein. The variant antibody or antigen-binding fragment may bind monomeric α-synuclein, e.g., as defined herein. The variant antibody or antigen-binding fragment may bind human monomeric α-synuclein, e.g., as defined herein. The variant antibody or antigen-binding fragment may bind aggregated α-synuclein, e.g., as defined herein. The variant antibody or antigen-binding fragment may bind aggregated human α-synuclein, e.g., as defined herein. The variant antibody or antigen-binding fragment may bind monomeric α-synuclein and may bind aggregated α-synuclein, e.g., as defined herein. The variant antibody or antigen-binding fragment may bind monomeric human α-synuclein and may bind aggregated human α-synuclein, e.g., as defined herein. The variant antibody or antigen-binding fragment used in the invention may bind human α-synuclein, rat α-synuclein and cynomolgus α-synuclein, e.g., as defined herein. The variant antibody or antigen-binding fragment may bind to the same epitope as the reference antibody or antigen-binding fragment thereof, e.g., as described herein. The variant antibody or antigen-binding fragment thereof may compete with the reference antibody (e.g., aslo0542 ngl-3) for binding to human α-synuclein, e.g., as described herein. The variant antibody or antigen-binding fragment may bind the C-terminal region (residues 96-140) of human α-synuclein, e.g., as defined herein. The variant antibody or antigen-binding fragment may bind the region comprised between about amino acid 102 and about amino acid 130 of human α-synuclein, e.g., as defined herein.

In particular, conservative amino acid substitutions are contemplated. Conservative replacements are those that take place within a family of amino acids that have related side chains. Genetically encoded amino acids are generally divided into families: (1) acidic: aspartate, glutamate; (2) basic: lysine, arginine, histidine; (3) non-polar: alanine, valine, leucine, isoleucine, proline, phenylalanine, methionine, tryptophan; and (4) uncharged polar: glycine, asparagine, glutamine, cysteine, serine, threonine, tyrosine. These families can be further categorised: serine and threonine are an aliphatic-hydroxy family; asparagine and glutamine are an amide-containing family; alanine, valine, leucine and isoleucine are an aliphatic family; and phenylalanine, tryptophan, and tyrosine are an aromatic family. Thus, in general one could expect that an isolated replacement of a leucine with an isoleucine or valine, an aspartate with a glutamate, a threonine with a serine, or a similar replacement of an amino acid with a structurally related amino acid, will not have a major effect on the binding function or properties of the resulting antibody, especially if the replacement does not involve an amino acid within a CDR site.

The anti-α-synuclein antibody or antigen-binding fragment thereof may be a variant antibody or antigen binding fragment thereof wherein the reference antibody is asyn0087 or an antigen binding fragment thereof comprising the VH of SEQ ID NO: 2 and the VL of SEQ ID NO: 3. In particular, the anti-α-synuclein antibody or antigen-binding fragment thereof may be a variant antibody or antigen binding fragment thereof wherein the reference antibody is asyn0087 or an antigen binding fragment thereof comprising the VH of SEQ ID NO: 2 and the VL of SEQ ID NO: 3, wherein said variant antibody or antigen-binding fragment has a K_{D} of less than 500 nM and binds the same epitope as any one of antibodies asyn0087, aslo0452ngl-3 and aslo0543, described herein.

The anti-α-synuclein antibody or antigen-binding fragment thereof may comprise a variable heavy chain region (VH) of amino acid sequence of SEQ ID NO: 2 and a variable light chain region (VL) of amino acid sequence SEQ ID NO: 3.

The anti-α-synuclein antibody or antigen-binding fragment thereof may comprise a variable heavy chain having a sequence defined by SEQ ID NO: 4 and a variable light chain having a sequence defined by SEQ ID NO: 8. The anti-α-synuclein antibody or antigen-binding fragment thereof may comprise a variable heavy chain having a sequence defined by SEQ ID NO: 4 and a variable light chain having a sequence defined by SEQ ID NO: 8, and bind to human α-synuclein with a K_{D} of less than 500 pM and binds the same epitope as asyn0087, aslo0452 ngl-3 or aslo0543.

The anti-α-synuclein antibody or antigen-binding fragment thereof may comprise at least one CDR selected from:
(i) H-CDR1 of SEQ ID NO: 5,
(ii) H-CDR2 of SEQ ID NO: 6,
(iii) H-CDR3 of SEQ ID NO: 7,
(iv) L-CDR1 of SEQ ID NO: 9,
(v) L-CDR2 of SEQ ID NO: 10,
(vi) L-CDR3 of SEQ ID NO: 11.
   For example, the anti-α-synuclein antibody or antigen-binding fragment thereof may comprise 1, 2, 3, 4, 5 or all 6 of the CDR sequences of SEQ ID NOs 5, 6, 7, 9, 10 and 11. The anti-α-synuclein antibody or antigen-binding fragment thereof may comprise H-CDR1 of SEQ ID NO: 5, H-CDR2 of SEQ ID NO: 6, H-CDR3 of SEQ ID NO: 7, L-CDR1 of SEQ ID NO: 9, L-CDR2 of SEQ ID NO: 10, and L-CDR3 of SEQ ID NO: 11. The anti-α-synuclein antibody or antigen-binding fragment thereof may comprise H-CDR3 of SEQ ID NO: 7.

The framework region and CDRs or an antibody may be precisely defined (see, Kabat et al. (1991) Sequences of Proteins of Immunological Interest, Fifth Edition, U.S. Department of Health and Human Services pp. 91-3242; and Chothia et al. (1987) J. Mol. Biol. 196: 901-917, both of which are incorporated herein by reference).

Minor variations in the amino acid sequences of an anti-α-synuclein antibody or antigen-binding fragment thereof are contemplated as being encompassed by the present invention, providing that the variations in the amino acid sequence(s) maintain at least 75%, more preferably at least 80%, at least 90%, at least 95%, and most preferably at least 99% sequence identity to equivalent sequence of the reference anti-α-synuclein antibody or antigen-binding fragment thereof as defined anywhere herein. In particular, conservative amino acid replacements are contemplated.

The invention also relates to the use of a single chain amino acid sequence comprising the light chain of anti-α-synuclein antibody or antigen-binding fragment thereof as defined anywhere herein. The invention also relates to the use of a single chain amino acid sequence comprising the heavy chain of an anti-α-synuclein antibody or antigen-binding fragment thereof as defined anywhere herein.

Optimal alignment of sequences for comparison may be conducted, for example, by the local homology alignment algorithm of Smith and Waterman (Smith and Waterman (1981) Adv. Appl. Math. 2: 484; incorporated herein by reference), by the algorithm of Needleman & Wunsch (Needleman and Wunsch (1970) J. Mol. Biol. 48: 443; incorporated herein by reference) by the search for similarity method of Pearson and Lipman (1988; Proc. Natl. Acad. Sci. USA 85: 2444; incorporated herein by reference), by computer implementations of these algorithms (GAP, BESTFIT, FASTA, and TFASTA-Sequence Analysis Software Package of the Genetics Computer Group, University of Wisconsin Biotechnology Center, 1710 University Avenue, Madison, Wis. 53705), or by visual inspection (see Current Protocols in Molecular Biology, F.M. Ausbel et al., eds., Current Protocols, a joint venture between Greene Publishing Associates, Inc. And John Wiley & Sons, Inc. (1995 Supplement); incorporated herein by reference).

Examples of algorithms suitable for determining percent sequence similarity or identity are the BLAST and BLAST 2.0 algorithms (see Altschul et al. (1990) J. Mol. Biol. 215(3): 403-410; and “http://www.ncbi.nlm.nih.gov/” of the National Center for Biotechnology Information; both of which are incorporated herein by reference).

The anti-α-synuclein antibody or antigen-binding fragment thereof as defined anywhere above may be an IgA, IgD, IgE, IgM, IgG such as IgG1, lgG2, lgG3, or lgG4 antibody or antigen-binding fragment thereof.

The anti-α-synuclein antibody or antigen-binding fragment thereof may not have an Fc region. Preferably, the anti-α-synuclein antibody or antigen-binding fragment thereof has an Fc region. The anti-α-synuclein antibody or antigen-binding fragment thereof may have a modified Fc region. Suitable modifications are well known to those skilled in the art and may include inter alia modifications to increase or decrease half-life, ablate, reduce or enhance effector function, provide substituted cysteines with free thiols for conjugation. Examples of such modifications are YTE to increase half-life and/or TM to reduce effector function. The anti-α-synuclein antibody or antigen-binding fragment thereof may have reduced binding affinity to IgG Fc receptors. Thus, the anti-α-synuclein antibody or antigen-binding fragment thereof may have a low immunogenic effect. Any of the antibodies or antigen-binding fragments disclosed herein may comprise mutations M252Y/S254T/T256E (YTE) in the Fc region of the antibody (Dall'Acqua et al. (2006) J. Biol. Chem. 281: 23514-23524). Any of the antibodies or antigen-binding fragments disclosed herein may comprise a triple mutation (abbreviated herein as “TM”) in the Fc region corresponding to the L234F/L235E/P331S mutation disclosed in Oganesyan et al. (2008) Acta Crystallogr. D. Biol. Crystallogr. 64: 700-704. The anti-α-synuclein antibody or antigen-binding fragment thereof may comprise a triple mutation in the Fc region corresponding to L234F/L235E/P331S numbered based on Kabat numbering. The anti-α-synuclein antibody or antigen-binding fragment thereof may be an lgG1 TM antibody or antigen-binding fragment thereof. IgG1 TM is a lgG1 triple mutant, which contains 3 point mutations (L234F/L235E/P331S) in the Fc domain that reduce the binding affinity of the antibody or antigen-binding fragment thereof to Fc-gamma receptors (FcyRs) (Oganesyan et al. (2008) Acta Crystallogr. D. Biol. Crystallogr. 64: 700-704; incorporated herein by reference). The antibody-mediated prevention of alpha-synuclein spreading may not require Fc-associated effector functions as a key mechanism of action. The anti-α-synuclein antibody or antigen-binding fragment thereof may comprise a Fc region having YTE mutations. The Fc region may be mutated or exchanged for alternative protein sequences or the anti-α-synuclein antibody or antigen-binding fragment thereof may be chemically modified to increase its blood brain barrier penetration.

The anti-α-synuclein antibody or antigen-binding fragment thereof may be linked to a half-life extender. As used herein, the term “half-life extender” means any molecule that, when linked to an anti-α-synuclein antibody or antigen-binding fragment thereof disclosed herein, increases the half-life of the anti-α-synuclein antibody or antigen-binding fragment thereof. Any half-life extender may be linked either covalently or non-covalently to the anti-α-synuclein antibody or antigen-binding fragment thereof. The half-life extender may be polyethylene glycol, a peptide, or human serum albumin.

The anti-α-synuclein antibody or antigen-binding fragment thereof may be associated with a blood brain barrier (BBB)-penetrable molecule. The term “BBB-penetrable molecule” means any molecule that, when linked to an anti-α-synuclein antibody or antigen-binding fragment thereof disclosed herein, increases the brain penetrating ability of the anti-α-synuclein antibody or antigen-binding fragment thereof. For example, a BBB-penetrable molecule may be a molecule that can specifically bind to brain microvascular endothelial cells (BMVEC) of one or more species and cross through BMVEC in vitro or in vivo, e.g., from the peripheral vasculature into the CNS vasculature. Whether a molecule is a BBB-penetrable molecule can be tested by a variety of in vitro or in vivo assays known to persons of ordinary skill in the art. For example, the transporter molecule can be tested in an in vitro transcytosis assay, in an in vivo assay such as a diuresis assay, as described in US 62/094,503. Other assays that could be used to measure in vivo delivery of payloads across the BBB include, without limitation, chronic constriction injury (CCI); spared nerve injury model (SNI) or spinal nerve ligation (SNL), all of which can be measured via paw flick, or the Hargreaves method (Hargreaves K, et al., Pain; 1988; 32; 77-88). In certain aspects, the BBB-penetrable molecule as provided herein can bind to BMVEC from one or more species, e.g., human, cynomolgus monkey, murine, rat, or bovine BMVEC. Binding can be demonstrated in various ways known to persons of ordinary skill in the art, e.g., in a FMAT assay as described in US 62/094,503. In certain aspects, the BMVEC are brain capillary endothelial cells (BCEC). In certain aspects, the BBB-penetrable molecule as provided herein can pass through a monolayer of BCEC in an in vitro transcytosis assay. In certain aspects, BBB-penetrable molecule activity can be demonstrated by visualization of the BBB-penetrable molecule in the CNS. For example, a tritium-labeled transporter molecule can be delivered to a subject, e.g., a mouse peripherally, e.g., intravenously, and then visualized in the CNS via quantitative whole body radiography. In certain aspects, the BBB-penetrable molecule localizes in specific regions of the CNS, e.g., the cortex of cerebellum, the gray matter of the cerebrum, the gray matter of the spinal cord, the pons, or a combination thereof.

The anti-α-synuclein antibody or antigen-binding fragment thereof may be isolated. The anti-α-synuclein antibody or antigen-binding fragment thereof may be purified.

The anti-α-synuclein antibody or antigen-binding fragment thereof may be a monoclonal antibody. The anti-α-synuclein antibody or antigen-binding fragment thereof may be a humanized antibody. The anti-α-synuclein antibody or antigen-binding fragment thereof may be a human antibody.

Antigen-binding fragments for use in the invention include Fab, Fv, scFv, dAb, Fd, Fab′, F(ab′)₂ or an isolated complementarity determining region (CDR) having sufficient framework to bind α-synuclein. A Fab fragment may be a monovalent fragment consisting of the VL, VH, CL and CH1 domains. A F(ab')2 fragment may be a bivalent fragment comprising two Fab fragments linked by a disulfide bridge at the hinge region. A Fc fragment may consist of the CH2 and CH3 domains. A Fv fragment may consist of the VL and VH domains of a single arm of an antibody. A dAb fragment (Ward et al. (1989) Nature 341: 544-546; incorporated herein by reference) may consist of a VH domain. An isolated complementarity determining region (CDR) having sufficient framework to bind may be an antigen binding portion of a variable region.

An antigen binding portion of a light chain variable region and an antigen binding portion of a heavy chain variable region, e.g., the two domains of the Fv fragment, VL and VH, can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242(4877): 423-426; and Huston et al. (1988) Proc. Natl. Acad. Sci. USA 85: 5879-5883; both of which are incorporated herein by reference). These are obtained using conventional techniques known to those with skill in the art, and the portions are screened for utility in the same manner as are intact antibodies.

Anti-α-synuclein antibodies or antigen-binding fragments thereof for use in the invention may have any or all of the advantageous properties as defined above or combinations thereof. In particular, anti-α-synuclein antibodies or antigen-binding fragments thereof for use in the invention may be selective for alpha-synuclein and be able to slow or prevent cell-to-cell transmission and spreading of alpha-synuclein in vivo.

The functionality of the anti-α-synuclein antibody or antigen-binding fragment thereof for use in the invention and in particular (i) its ability to bind the epitope of alpha-synuclein; and (ii) its ability to slow or prevent cell-to-cell transmission and spreading of alpha-synuclein in vivo can readily be determined by assaying its specific activity using the techniques described in WO2017/207739, which is hereby incorporated by reference in its entirety.

### 4.3 Pharmaceutical Compositions and Kits

The anti-α-synuclein antibody or antigen-binding fragment thereof may be provided in a pharmaceutical composition. In accordance with the invention, the anti-α-synuclein antibody or antigen-binding fragment thereof may be administered in the form of a pharmaceutical composition. Suitable pharmaceutical compositions may comprise an anti-α-synuclein antibody or antigen-binding fragment thereof as defined anywhere herein together with a pharmaceutically acceptable excipient.

The phrase “pharmaceutically acceptable excipient” includes any and all solvents, dispersion media, coatings, antibacterial agents and antifungal agents, isotonic agents, and absorption delaying agents, and the like, that are compatible with pharmaceutical administration. The compositions may also contain other active compounds providing supplemental, additional, or enhanced therapeutic functions. The pharmaceutical compositions may also be included in a container, pack, or dispenser together with instructions for administration.

The present invention also relates to pharmaceutical compositions comprising a fixed dose of 50-5,000 mg (or any other fixed dose as described herein) of an anti-α-synuclein antibody or antigen-binding fragment thereof. The anti-α-synuclein antibody or antigen-binding fragment thereof may be any anti-α-synuclein antibody or antigen-binding fragment thereof as described herein. Accordingly, the present invention provides a pharmaceutical composition comprising a fixed dose of 50-5,000 mg (or any other fixed dose as described herein) of an anti-α-synuclein antibody or antigen-binding fragment thereof, together with a pharmaceutically acceptable excipient. Suitable pharmaceutically acceptable excipients may facilitate processing of the active compounds into preparations suitable for pharmaceutical administration. The present invention provides a unit dosage form of a pharmaceutical composition comprising a fixed dose of 50-5,000 mg (or any other fixed dose as described herein) of an anti-α-synuclein antibody or antigen-binding fragment thereof, together with a pharmaceutically acceptable excipient. The present invention also provides a unit dosage form of 50-5,000 mg (or any other fixed dose as described herein) of an anti-α-synuclein antibody or antigen-binding fragment thereof. As used herein, a “unit dosage form” refers to an amount of a medication e.g. an anti-α-synuclein antibody or antigen-binding fragment thereof, which is to be administered to a subject in a single dose.

A pharmaceutical composition as disclosed herein is formulated to be compatible with its intended route of administration. The administration may, for example, be intravenous, intraperitoneal, intramuscular, intracavity, subcutaneous, intradermal or transdermal.

The present invention also relates to a kit comprising an anti-α-synuclein antibody, or antigen-binding fragment thereof (which may be any anti-α-synuclein antibody, or antigen-binding fragment thereof as described herein) and optionally instructions for use of the same for treating an α-synucleinopathy at a dose of 50-5,000 mg. Preferably, the instructions are instructions for use of the anti-α-synuclein antibody, or antigen-binding fragment thereof for treating an α-synucleinopathy at a fixed dose of 50-5,000 mg (or any other fixed dose as described herein).

The kit may comprise 50-5,000 mg (or any other fixed dose as described herein) of the anti-α-synuclein antibody, or antigen-binding fragment thereof. The anti-α-synuclein antibody, or antigen-binding fragment thereof may be aslo0452 ngl-3.

Pharmaceutical compositions as disclosed herein may be formulated for, but not limited to parenteral delivery, for example intramuscular, subcutaneous or intravenous. Compositions suitable for intramuscular, subcutaneous or intravenous injection include sterile aqueous solutions.

The pharmaceutical composition may take the form of an aqueous solution and may include physiologically compatible buffers such as Hank's solution, Ringer's solution, or physiologically buffered saline. The pharmaceutical composition may additionally or alternatively contain substances which increase the viscosity of the suspension, such as sodium carboxymethyl cellulose, sorbitol, or dextran. The pharmaceutical composition may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the pharmaceutical composition may contain suitable stabilisers or agents which increase the solubility of the compounds to allow for the preparation of highly concentrated solutions.

Informal sequence listing

| | SEQ ID | |
|---|---|---|
| Description | NO. | Sequence |
| Human α- | 1 | MDVFMKGLSKAKEGVVAAAEKTKQGVAEAAGKTKEG |
| synuclein | | VLYVGSKTKEGVVHGVATVAEKTKEQVTNVGGAVVT |
| | | GVTAVAQKTVEGAGSIAAATGFVKKDQLGKNEEGAP |
| | | QEGILEDMPVDPDNEAYEMPSEEGYQDYEPEA |
| Asyn0087 VH | 2 | EVQLLESGGGLVQPGGSLRLSCAASGFTFSSYAMSW |
| | | VRQAPGKGLEWVSAISGSGGSTYYADSVKGRFTISR |
| | | DNSKNTLYLQMNSLRAEDTAVYYCAGGAVNVYYYYG |
| | | MDVWGQGTMVTVSS |
| Asyn0087 VL | 3 | QAVLTQPSSLSASPGASASLTCTLRSGNNVGNYRIY |
| | | WYQQKSGSPPQYLLRYKSDADKHQGSGVPSRFSGSK |
| | | DASANAGILFISGLQSEDEADYYCMVWHSGAWVFGG |
| | | GTKLTVL |
| General | 4 | EVQLLESGGGLVQPGGSLRLSCAASGFTFSSYAMSW |
| sequence of a | | VRQAPGKGLEWVSXISXXGGSTYYADSVKGRFTISR |
| VH derived from | | DNSKNTLYLQMNSLRAEDTAVYYCAGGAXXXXXYYG |
| asyn0087 | | MDVWGQGTMVTVSS |
| | | X50 = S or A; X53 = H or G; X54 = L |
| | | or S; X101 = N or R; X102 = H or R; |
| | | X103 = V or G; X104 = K or R; X105 = |
| | | Y or I |
| General | 5 | SYAMS |
| sequence of a | | |
| H-CDR1 derived | | |
| from asyn0087 | | |
| General | 6 | XISXXGGSTYYADSVK |
| sequence of a | | X1 = S or A; X4 = H or G; X5 = L or |
| H-CDR2 derived | | S |
| from asyn0087 | | |
| General | 7 | GAXXXXXYYGMDV |
| sequence of a | | X3 = N or R; X4 = H or R; X5 = V or |
| H-CDR3 derived | | G; X6 = K or R; X7 = Y or I |
| from asyn0087 | | |
| General | 8 | QAVLTQPSSLSASPGASASLTCTLRSXXXXXXYRIY |
| sequence of a | | WYQQKXGSPPQYLLRYKSDADKHQGSGVPSRFSGSK |
| VL derived from | | DASANAGILXSGLQSEDEADYYCMVWXXGXWXFGGG |
| asyn0087 | | TKLTVL |
| | | X27 = G or S; X28 = A or G; X29 = P |
| | | or D; X30 = L or F; X31 = P or S; |
| | | X32 = K or R; X42 = S or P; X82 = F |
| | | or L; X99 = D or S; X100 = H or S; |
| | | X102 = V or A; X104 = V or Y |
| General | 9 | TLRSXXXXXXYRIY |
| sequence of a L- | | X5 = G or S; X6 = A or G; X7 = P or |
| CDR1 derived | | D; X8 = L or F; X9 = P or S; X10 = |
| from asyn0087 | | K or R |
| General | 10 | YKSDADKHQGS |
| sequence of a L- | | |
| CDR2 derived | | |
| from asyn0087 | | |
| General | 11 | MVWXXGXWX |
| sequence of a L- | | X4 = D or S; X5 = H or S; X7 = V or |
| CDR3 derived | | A; X9 = V or Y |
| from asyn0087 | | |
| Aslo0452 ngl-3 | 12 | EVQLLESGGGLVQPGGSLRLSCAASGFTFSSYAMSW |
| heavy chain | | VRQAPGKGLEWVSSISHLGGSTYYADSVKGRFTISR |
| | | DNSKNTLYLQMNSLRAEDTAVYYCAGGANHGKYYYG |
| | | MDKWGQGTTVTVSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKV |
| | | DKRVEPKSCDKTHTCPPCPAPEFEGGPSVFLFPPKP |
| | | KDTLMISRTPEVTCVVVDVSHEDPEVKFNWYVDGVE |
| | | VHNAKTKPREEQYNSTYRVVSVLTVLHQDWLNGKEY |
| | | KCKVSNKALPASIEKTISKAKGQPREPQVYTLPPSR |
| | | EEMTKNQVSLTCLVKGFYPSDIAVEWESNGQPENNY |
| | | KTTPPVLDSDGSFFLYSKLTVDKSRWQQGNVFSCSV |
| | | MHEALHNHYTQKSLSLSPGK |
| Aslo0452 ngl-3 | 13 | GAGGTGCAGCTGTTGGAGTCTGGGGGAGGCTTGGTA |
| VH nucleotide | | CAGCCTGGGGGGTCCCTGAGACTCTCCTGTGCAGCC |
| sequence | | TCTGGATTCACCTTTAGCAGCTATGCCATGAGCTGG |
| | | GTCCGCCAGGCTCCAGGGAAGGGGCTGGAGTGGGTC |
| | | TCATCCATTTCCCACCTTGGTGGTAGCACATACTAC |
| | | GCAGACTCCGTGAAGGGCCGGTTCACCATCTCCAGA |
| | | GACAATTCCAAGAACACGCTGTATCTGCAAATGAAC |
| | | AGCCTGAGAGCCGAGGACACGGCCGTGTATTACTGT |
| | | GCGGGAGGGGCAAACCACGGGAAGTACTACTACGGA |
| | | ATGGACAAGTGGGGCCAAGGGACCACGGTCACCGTC |
| | | TCCTCA |
| Aslo0452 ngl-3 | 14 | EVQLLESGGGLVQPGGSLRLSCAASGFTFSSYAMSW |
| VH | | VRQAPGKGLEWVSSISHLGGSTYYADSVKGRTISRD |
| | | NSKNTLYLQMNSLRAEDTAVYYCAGGANHGKYYYGM |
| | | DKWGQGTTVTVSS |
| Aslo0452 ngl-3 | 5 | SYAMS |
| H-CDR1 | | |
| Also0452 ngl-3 | 15 | SISHLGGSTYYADSVKG |
| H-CDR2 | | |
| Aslo0452 ngl-3 | 16 | GANHGKYYYGMDK |
| H-CDR3 | | |
| Aslo0452 ngl-3 | 17 | QAVLTQPASLSASPGASASLTCTLRSGAPLPKYRIY |
| light chain | | WYQQKPGSPPQYLLRYKSDADKHQGSGVPSRFSGSK |
| | | DASANAGILLISGLQSEDEADYYCMVWDHGVWYFGG |
| | | GTKLTVLGQPKAAPSVTLFPPSSEELQANKATLVCL |
| | | ISDFYPGAVTVAWKADSSPVKAGVETTTPSKQSNNK |
| | | YAASSYLSLTPEQWKSHRSYSCQVTHEGSTVEKTVA |
| | | PTECS |
| Also0452 ngl-3 | 18 | CAGGCTGTGCTGACTCAGCCGGCTTCCCTCTCTGCG |
| VL nucleotide | | TCTCCTGGAGCATCAGCCAGTCTCACCTGCACCTTG |
| sequence | | CGCAGTGGGGCGCCCCTGCCGAAGTATAGGATATAC |
| | | TGGTATCAGCAGAAGCCAGGGAGTCCTCCCCAGTAT |
| | | CTCCTGAGGTACAAATCAGACGCAGATAAACACCAG |
| | | GGCTCTGGAGTCCCCAGCCGCTTTTCTGGATCCAAA |
| | | GATGCTTCGGCCAATGCAGGGATTTTACTCATCTCT |
| | | GGGCTCCAGTCTGAGGATGAGGCTGACTATTATTGT |
| | | ATGGTTTGGGACCACGGCGTCTGGTATTTCGGCGGA |
| | | GGGACCAAGCTGACCGTCCTA |
| Also0452 ngl-3 | 19 | QAVLTQPASLSASPGASASLTCTLRSGAPLPKYRIY |
| VL | | WYQQKPGSPPQYLLRYKSDADKHQGSGVPSRFSGSK |
| | | DASANAGILLISGLQSEDEADYYCMVWDHGVWYFGG |
| | | GTKLTVL |
| Also0452 ngl-3 | 20 | TLRSGAPLPKYRIY |
| L-CDR1 | | |
| Aslo0452 ngl-3 | 10 | YKSDADKHQGS |
| L-CDR2 | | |
| Aslo0452 ngl-3 | 21 | MVWDHGVWY |
| L-CDR3 | | |
| Aslo0543 heavy | 22 | EVQLLESGGGLVQPGGSLRLSCAASGFTFSSYAMSW |
| chain | | VRQAPGKGLEWVSAISGSGGSTYYADSVKGRFTISR |
| | | DNSKNTLYLQMNSLRAEDTAVYYCAGGARRGRIYYG |
| | | MDKWGQGTTVTVSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKV |
| | | DKRVEPKSCDKTHTCPPCPAPEFEGGPSVFLFPPKP |
| | | KDTLMISRTPEVTCVVVDVSHEDPEVKFNWYVDGVE |
| | | VHNAKTKPREEQYNSTYRVVSVLTVLHQDWLNGKEY |
| | | KCKVSNKALPASIEKTISKAKGQPREPQVYTLPPSR |
| | | EEMTKNQVSLTCLVKGFYPSDIAVEWESNGQPENNY |
| | | KTTPPVLDSDGSFFLYSKLTVDKSRWQQGNVFSCSV |
| | | MHEALHNHYTQKSLSLSPGK |
| Aslo0543 VH | 23 | GAGGTGCAGCTGTTGGAGTCTGGGGGAGGCTTGGTA |
| nucleotide | | CAGCCTGGGGGGTCCCTGAGACTCTCCTGTGCAGCC |
| sequence | | TCTGGATTCACCTTTAGCAGCTATGCCATGAGCTGG |
| | | GTCCGCCAGGCTCCAGGGAAGGGGCTGGAGTGGGTC |
| | | TCAGCTATTAGTGGTAGTGGTGGTAGCACATACTAC |
| | | GCAGACTCCGTGAAGGGCCGGTTCACCATCTCCAGA |
| | | GACAATTCCAAGAACACGCTGTATCTGCAAATGAAC |
| | | AGCCTGAGAGCCGAGGACACGGCCGTGTATTACTGT |
| | | GCGGGAGGGGCACGGCGCGGGCGCATCTACTACGGA |
| | | ATGGACAAATGGGGCCAAGGGACAACGGTCACCGTC |
| | | TCCTCA |
| Aslo0543 VH | 24 | EVQLLESGGGLVQPGGSLRLSCAASGFTFSSYAMSW |
| | | VRQAPGKGLEWVSAISGSGGSTYYADSVKGRFTISR |
| | | DNSKNTLYLQMNSLRAEDTAVYYCAGGARRGRIYYG |
| | | MDKWGQGTTVTVSS |
| Aslo0543 H- | 25 | SYAMS |
| CDR1 | | |
| Aslo0543 H- | 26 | AISGSGGSTYYADSVKG |
| CDR2 | | |
| Aslo0543 H- | 27 | GARRGRIYYGMDK |
| CDR3 | | |
| Aslo0543 light | 28 | QAVLTQPASLSASPGASASLTCTLRSSGDFSRYRIY |
| chain | | WYQQKPGSPPQYLLRYKSDADKHQGSGVPSRFSGSK |
| | | DASANAGILLISGLQSEDEADYYCMVWSSGAWYFGG |
| | | GTKLTVLGQPKAAPSVTLFPPSSEELQANKATLVCL |
| | | ISDFYPGAVTVAWKADSSPVKAGVETTTPSKQSNNK |
| | | YAASSYLSLTPEQWKSHRSYSCQVTHEGSTVEKTVA |
| | | PTECS |
| Aslo0543 VL | 29 | CAGGCTGTGCTGACTCAGCCGGCTTCCCTCTCTGCG |
| nucleotide | | TCTCCTGGAGCATCAGCCAGTCTCACCTGCACCTTG |
| sequence | | CGCAGTTCCGGGGACTTCTCCCGGTATAGGATATAC |
| | | TGGTATCAGCAGAAGCCAGGGAGTCCTCCCCAGTAT |
| | | CTCCTGAGGTACAAATCAGACGCAGATAAACACCAG |
| | | GGCTCTGGAGTCCCCAGCCGCTTTTCTGGATCCAAA |
| | | GATGCTTCGGCCAATGCAGGGATTTTACTCATCTCT |
| | | GGGCTCCAGTCTGAGGATGAGGCTGACTATTATTGT |
| | | ATGGTTTGGTCCAGCGGCGCTTGGTACTTCGGCGGA |
| | | GGGACCAAGCTGACCGTCCTA |
| Aslo0543 VL | 30 | QAVLTQPASLSASPGASASLTCTLRSSGDFSRYRIY |
| | | WYQQKPGSPPQYLLRYKSDADKHQGSGVPSRFSGSK |
| | | DASANAGILLISGLQSEDEADYYCMVWSSGAWYFGG |
| | | GTKLTVL |
| Aslo0543 L- | 31 | TLRSSGDFSRYRIY |
| CDR1 | | |
| Aslo0543 L- | 32 | YKSDADKHQGS |
| CDR2 | | |
| Aslo0543 L- | 33 | MVWSSGAWY |
| CDR3 | | |
| General BBB | 34 | XVQLVQSGAEXKKPGSSVKVSCKASGGTFSSYAISW |
| transporter VH | | VRQAPGQGLEWMGRIIPILGTSNYAQKFQGRVTITA |
| | | DEXTSTAYMELSSLRSEDTAVYYCARRSSLAAADRG |
| | | AFDIWGQGTMVTVSS |
| | | X1 = Q or V; X11 = V or L; X75 = S |
| | | or R |
| General BBB | 35 | SSELTQDPAVSVALXQTVRITCQGDSLXXYYXXWYQ |
| transporter VL | | XKPGQAPXLVXYGXXNRPSGXPDRFSGSXSGXTASL |
| | | TITGAQAEDEADYYCNSRDXXGXXXVFGGGTKLTVL |
| | | X15 = R, G or A; X28 = T or R; X29 = |
| | | S, R or T; X32 = A or T; X33 = N or |
| | | S; X37 = H or Q; X44 = V or I; X47 = |
| | | M or I; X50 = K or E; X51 = N or D; |
| | | X57 = V or I; X65 = S or R; X68 = N |
| | | or T; X92 = S or N; X93 = S or T; |
| | | X95 = N, K or H; X96 = H or P; X97 = |
| | | V or W |
| General BBB | 36 | QGDSLXXYYXX |
| transporter L- | | X6 = T or R; X7 = S, R or T; X10 = A |
| CDR1 | | or T; X11 = N or S |
| General BBB | 37 | GXXNRPS |
| transporter L- | | X2 = K or E; X3 = N or D |
| CDR2 | | |
| General BBB | 38 | NSRDXXGXXXV |
| transporter L- | | X5 = S or N; X6 = S or T; X8 = N, K |
| CDR3 | | or H; X9 = H or P; X10 = V or W |
| BBBt0626gl VH | 39 | QVQLVQSGAEVKKPGSSVKVSCKASGGTFSSYAISW |
| | | VRQAPGQGLEWMGRIIPILGTSNYAQKFQGRVTITA |
| | | DESTSTAYMELSSLRSEDTAVYYCARRSSLAAADRG |
| | | AFDIWGQGTMVTVSS |
| BBBt0626gl H- | 40 | SYAIS |
| CDR1 | | |
| BBBt0626gl H- | 41 | RIIPILGTSNYAQKFQG |
| CDR2 | | |
| BBBt0626gl H- | 42 | RSSLAAADRGAFDI |
| CDR3 | | |
| BBBt0626gl VL | 43 | SSELTQDPAVSVALGQTVRITCQGDSLRSYYASWYQ |
| | | QKPGQAPVLVIYGKNNRPSGIPDRFSGSSSGNTASL |
| | | TITGAQAEDEADYYCNSRDSSGNHVVFGGGTKLTVL |
| BBBt0626gl L- | 44 | QGDSLRSYYAS |
| CDR1 | | |
| BBBt0626gl L- | 45 | GKNNRPS |
| CDR2 | | |
| BBBt0626gl L- | 46 | NSRDSSGNHVV |
| CDR3 | | |
| BBBt0626 VH | 47 | GVQLVQSGAELKKPGSSVKVSCKASGGTFSSYAISW |
| | | VRQAPGQGLEWMGRIIPILGTSNYAQKFQGRVTITA |
| | | DERTSTAYMELSSLRSEDTAVYYCARRSSLAAADRG |
| | | AFDIWGQGTMVTVSS |
| BBBt0626 H- | 40 | SYAIS |
| CDR1 | | |
| BBBt0626 H- | 41 | RIIPILGTSNYAQKFQG |
| CDR2 | | |
| BBBt0626 H- | 42 | RSSLAAADRGAFDI |
| CDR3 | | |
| BBBt0626 VL | 43 | SSELTQDPAVSVALGQTVRITCQGDSLRSYYASWYQ |
| | | QKPGQAPVLVIYGKNNRPSGIPDRFSGSSSGNTASL |
| | | TITGAQAEDEADYYCNSRDSSGNHVVFGGGTKLTVL |
| BBBt0626 L- | 44 | QGDSLRSYYAS |
| CDR1 | | |
| BBBt0626 L- | 45 | GKNNRPS |
| CDR2 | | |
| BBBt0626 L- | 46 | NSRDSSGNHVV |
| CDR3 | | |
| BBBt0632gl VH | 48 | QVQLVQSGAEVKKPGSSVKVSCKASGGTFGTYSITW |
| | | VRQAPGQGLEWMGDIVPIFGTPNYAQNFQGRVTISA |
| | | DVSTSTVYMELSSLRSEDTAVYYCAKRGSYYGRGGW |
| | | FDPWGRGTLVTVSS |
| BBBt0632gl H- | 49 | TYSIT |
| CDR1 | | |
| BBBt0632gl H- | 50 | DIVPIFGTPNYAQNFQG |
| CDR2 | | |
| BBBt0632gl H- | 51 | RGSYYGRGGWFDP |
| CDR3 | | |
| BBBt0632gl VL | 52 | QVQLVQSGAEVKKPGSSVKVSCKASGGTFGTYSITW |
| | | VRQAPGQGLEWMGDIVPIFGTPNYAQNFQGRVTISA |
| | | DVSTSTVYMELSSLRSEDTAVYYCAKRGSYYGRGGW |
| | | FDPWGRGTLVTVSS |
| BBBt0632gl L- | 53 | TYSIT |
| CDR1 | | |
| BBBt0632gl L- | 54 | DIVPIFGTPNYAQNFQG |
| CDR2 | | |
| BBBt0632gl L- | 55 | RGSYYGRGGWFDP |
| CDR3 | | |
| GGGGS linker | 56 | (GGGGS)₁₋₁₀ |
| GGGGS linker | 57 | GGGGSGGGGSGGGGS |
| Germline IGHV3- | 58 | EVQLLESGGGLVQPGGSLRLSCAASGFTFSSYAMSW |
| 23 | | VRQAPGKGLEWVSAISGSGGSTYYADSVKGRFTISR |
| | | DNSKNTLYLQMNSLRAEDTAVYYCAK |
| Germline JH6 | 59 | WGQGTTVTVSS |
| Germline IGLV5- | 60 | QAVLTQPASLSASPGASASLTCTLRSGINVGTYRIY |
| 45 | | WYQQKPGSPPQYLLRYKSDSDKQQGSGVPSRFSGSK |
| | | DASANAGILLISGLQSEDEADYYCMIWHWV |
| Germline JL2 | 61 | FGGGTKLTVL |
| Aslo0452 ngl-3 | 62 | EVQLLESGGGLVQPGGSLRLSCAASGFTFSSYAMSW |
| heavy chain | | VRQAPGKGLEWVSSISHLGGSTYYADSVKGRFTISR |
| | | DNSKNTLYLQMNSLRAEDTAVYYCAGGANHGKYYYG |
| | | MDKWGQGTTVTVSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKV |
| | | DKRVEPKSCDKTHTCPPCPAPEFEGGPSVFLFPPKP |
| | | KDTLMISRTPEVTCVVVDVSHEDPEVKFNWYVDGVE |
| | | VHNAKTKPREEQYNSTYRVVSVLTVLHQDWLNGKEY |
| | | KCKVSNKALPASIEKTISKAKGQPREPQVYTLPPSR |
| | | EEMTKNQVSLTCLVKGFYPSDIAVEWESNGQPENNY |
| | | KTTPPVLDSDGSFFLYSKLTVDKSRWQQGNVFSCSV |
| | | MHEALHNHYTQKSLSLSPG |

### EXAMPLES

### 5.1 Example 1: Effects of Different Doses of aslo0452 ngl-3 in Humans

### 5.1.1 Example 1.1: Effects of Single Doses of aslo0452 ngl-3 in Healthy Humans

Aslo0452 ngl-3 was obtained using the methods described in WO2017/207739 A1 which is incorporated by reference in its entirety herein.

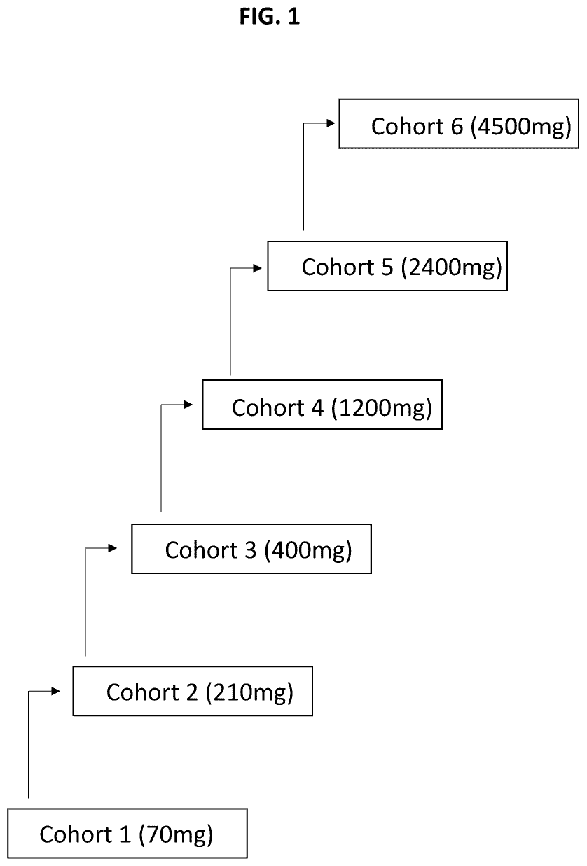

A randomized, double-blind, placebo-controlled study of single ascending doses (SAD) of from 70 mg up to 4,500 mg aslo0452 ngl-3 was designed for healthy human subjects aged 18 to 65 years. The SAD cohorts 1-6 included 8 subjects each, with six in each cohort receiving aslo0452 ngl-3 and two in each cohort receiving placebo. A simplified layout of the study design is provided in FIG. 1.

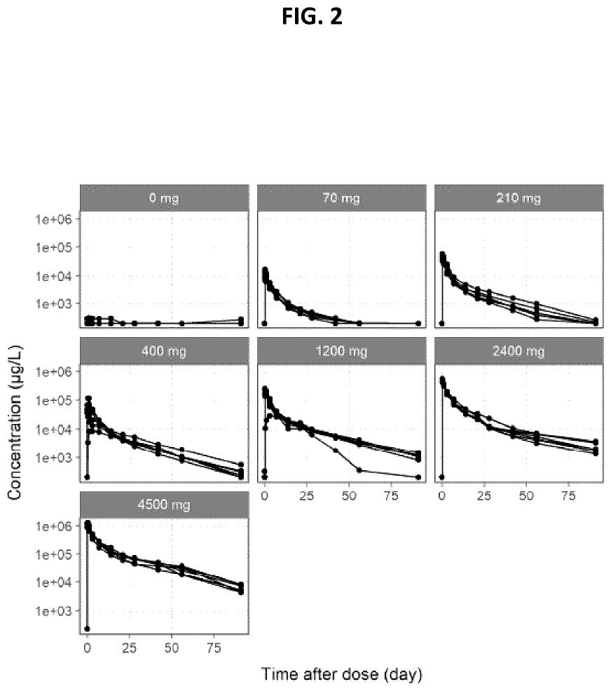

Subjects received either a single intravenous (IV) infusion of aslo0452 ngl-3 (70, 210, 400, 1200, 2400 or 4500 mg) or placebo. Cₘₐₓ values of aslo0452 ngl-3 were generally observed at the end of the 1-hour infusion; median tₘₐₓ was approximately 1 hour after start of the infusion at the 70, 210 and 2400 mg dose levels, 17 hours at the 400 mg dose level, 5 hours at the 4500 mg dose level, and 9 hours at the 1200 mg dose level. After the end of the intravenous infusion, serum concentrations of aslo0452 ngl-3 declined in a generally multi-phasic manner. Aslo0452 ngl-3 exposure increased in a dose-dependent manner across the dose range as shown in FIG. 2. The increase in aslo0452 ngl-3 dose from 1,200 to 2,400 mg and from 2,400 to 4,500 mg resulted in a greater than dose-proportional increase in Cₘₐₓ and AUC∞ (Table 1). Terminal elimination half-lives were comparable across the single dose levels (16.6 to 24.3 days).

TABLE 1
Geometric Mean (% CV) Serum PK Parameters for Aslo0452
ngl-3 in Phase 1 SAD Study in Healthy Subjects

| | Cₘₐₓ | AUC_{∞} | T_{1/2z} |
|---|---|---|---|
| Dose | (μg/mL) | (μg*day/mL) | (day) |
| 70 mg | 11.5 (30.4) | 64.1 (14.6) | 17.4 (27.1) |
| 210 mg | 43.1 (19.3) | 222 (24.2) | 16.6 (16.9) |
| 400 mg | 39.6 (102.8) | 386 (29.8) | 17.3 (13.2) |
| 1200 mg | 149 (96.1) | 1110 (19.8) | 17.3 (49.1) |
| 2400 mg | 462 (9.2) | 2590 (11.3) | 24.3 (20.8) |
| 4500 mg | 1130 (13.4) | 7630 (15.3) | 18.9 (13.2) |

% CV: percentage coefficient of variation; AUC_{∞}: area under the concentration-time curve from time 0 to infinity; Cₘₐₓ: maximum observed concentration; PK: pharmacokinetic; t_{1/2z}: terminal disposition phase half-life.

Free levels of α-synuclein (αSYN) in the cerebrospinal fluid (CSF) were used to determine the drug pharmacodynamic (PD) effects. Free αSYN protein was quantified in the CSF by a Mesoscale Discovery enzyme-linked immunosorbent assay (ELISA) after an immunoprecipitation procedure to remove αSYN bound to aslo0452 ngl-3. The percentage of CSF αSYN reduction are summarised for each dose in Table 2 below and represented as a mean change from a pre-dose baseline.

TABLE 2
Geometric mean (% CV) CSF α-Synuclein Reductions After
Aslo0452 ngl-3 in Phase I SAD Study in Healthy Subjects

| | Days from | % reduction in CSF | |
|---|---|---|---|
| Dose (mg) | first dose | free αSYN from baseline | |
| Placebo | 15 | 3.8 | (15.4) |
| 70 | 29 | 3.9 | (6.7) |
| 210 | 29 | 2.8 | (9.7) |
| 400 | 15 | 23.7 | (5.7) |
| 1200 | 15 | 25.3 | (20.4) |
| 2400 | 15 | 28.5 | (8.8) |
| 4500 | 15 | 53.7 | (9.2) |

% CV: percentage coefficient of variation;
CSF: cerebrospinal fluid;
SAD: single-ascending dose;
values in brackets are standard deviation

### 5.1.2 Example 1.2: Effects of Multiple Doses of aslo0452 ngl-3 in Parkinson's Disease Patients

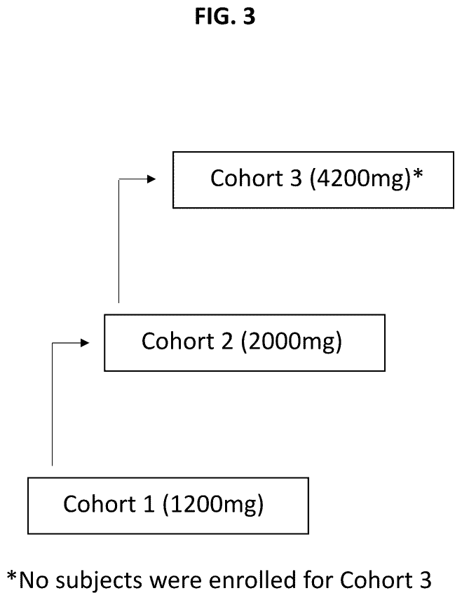

A multicenter, randomized, double-blind, placebo-controlled study of multiple ascending doses (MAD) of 1,200 mg, 2,000 mg, or 4,200 mg aslo0452 ngl-3 was designed for subjects aged 40 to 85 years of age, with Parkinson's Disease (PD). MAD cohorts 1-3 included 12 subjects each, with nine in each cohort receiving aslo0452 ngl-3 and three in each cohort receiving placebo. A simplified layout of the study design is provided in FIG. 3.

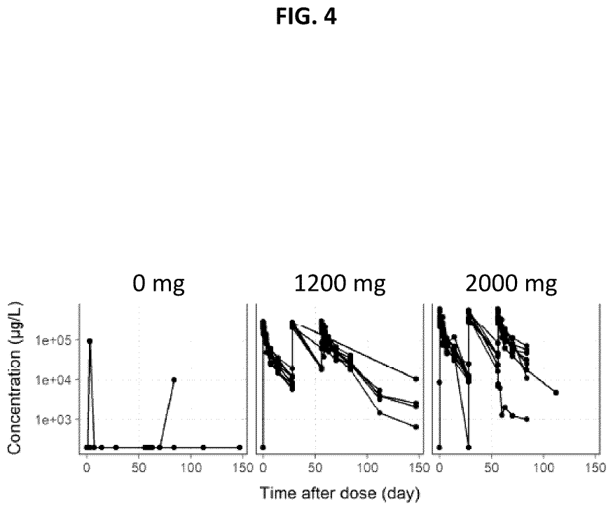

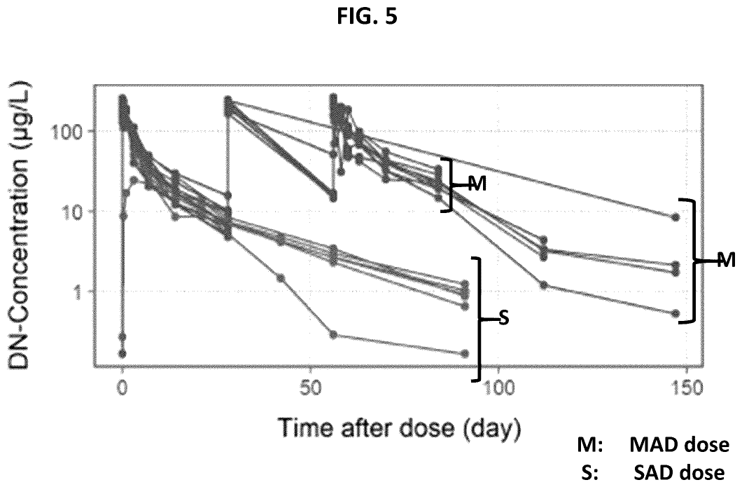

Subjects in MAD cohorts 1 and 2 received three intravenous infusions of aslo0452 ngl-3 or placebo, with 4 weeks between infusions (1 infusion on each of Days 1, 29, and 57). Cohort 3 (4,200 mg) was not enrolled. Following multiple intravenous infusions of 1200 mg also0452 ngl-3, Cₘₐₓ values of aslo0452 ngl-3 were generally observed at the end of the 1-hour infusion and median tₘₐₓ was approximately 1 hour after start of infusion for both the 1200 mg and 2000 mg multiple dose levels. After the end of intravenous infusion, serum concentrations of aslo0452 ngl-3 declined in a generally multi-phasic manner. The aslo0452 ngl-3 exposure increased in a dose-dependent manner across the dose range explored as shown in FIG. 4. All concentration time profiles from healthy subjects in the SAD trial described above were on the lower part of the observed distribution of concentration time profiles from PD patients as shown in FIG. 5. Terminal elimination half-lives were slightly shorter (14.0 days) than in the SAD trial. Between-subject variability as measured by geometric mean percentage coefficient of variation (% CV) was low to moderate for AUCs (11.3% to 29.8%) and low to high for Cₘₐₓ (0.2% to 102.8%) across all dose levels in the SAD and MAD trials.

Free levels of αSYN in the cerebrospinal fluid (CSF) were used to determine the drug PD effects. Free αSYN protein was quantified in the CSF by a Mesoscale Discovery ELISA after an immunoprecipitation procedure to remove αSYN bound to aslo0452 ngl-3. Multiple intravenous doses of aslo0452 ngl-3 of either 1,200 mg or 2,000 mg reduced CSF levels of αSYN. Mean change from baseline in CSF free αSYN levels is shown in Table 3 below. In general, free α-synuclein in CSF decreased notably for the aslo0452 ngl-3 treatment groups compared to placebo and the percentage change of α-synuclein from baseline appeared to be dose-dependent.

TABLE 3
Reduction from baseline in CSF Free α-Synuclein

| | Days after final | Mean change from baseline |
|---|---|---|
| Dose | injection | in CSF free αSYN |
| Placebo | 4 | −7.1% |
| | 28 | +12.6% |
| 1,200 mg | 4 | −55.5% |
| | 28 | −51.1% |
| 2,000 mg | 4 | −75.2% |
| | 28 | −59.0% |

Aslo0452 ngl-3 appeared to be safe and well tolerated in the SAD and MAD trials. There were no clinically significant abnormal values/changes in laboratory assessments, vital signs, and ECGs. Positive anti-drug antibody (ADA) results were observed across single dose levels. None of the positive ADA results appeared to have impacted PK and PD parameters and are not thought to have impacted safety parameters.

### 5.1.3 Example 1.3: Effects of Multiple Doses of aslo0452 ngl-3 in Multiple System Atrophy Patients

In view of the surprisingly good PK characteristics, target engagement, and the absence of safety issues, a multicenter, randomized, double-blind, placebo-controlled phase 2 study to test the efficacy, safety, tolerability, PK and PD of aslo0452 ngl-3 administered as multiple intravenous infusions every 4 weeks over 52 weeks was designed for subjects aged at least 40 years with possible or probable multiple system atrophy (MSA).

The study comprises a screening period of up to 42 days (6 weeks), a 52-week double-blind treatment period, and a follow-up safety visit approximately 90 days after the final infusion. Each subject will receive a total of 13 (IV infusions of aslo0452 ngl-3 of approximately 60 minutes each or placebo during the treatment period, with approximately 4 weeks between infusions, i.e., Q4W dosing. An early PK cohort consisting of the first approximately 15 subjects will be randomized 2:1 to receive either aslo0452 ngl-3 or placebo (approximately 10 subjects on aslo0452 ngl-3 and 5 subjects on placebo). Dosing of subjects in the early PK cohort will be initiated at 2400 mg Q4W. After the early PK cohort is fully enrolled, further subjects will be enrolled into the main cohort. Subjects in the main cohort will receive 2000 mg aslo0452 ngl-3 while analysis of PK data from the early PK cohort is ongoing. Depending on the PK, safety, immunogenicity, and tolerability data from the early PK cohort, the aslo0452 ngl-3 dose level may be adjusted for subjects receiving active treatment.

The total Unified Multiple System Atrophy Rating Scale (UMSARS) will be determined for all subjects. The primary objective is to evaluate the change from baseline in a modified UMSARS Part I at week 52.

### Secondary Objectives:

The secondary objectives include:
a) to assess the serum PK and CSF concentrations of aslo0452 ngl-3 in subjects with MSA;
b) to evaluate the efficacy of aslo0452 ngl-3 versus placebo, as measured by the change from baseline to Week 52 on the 11-item UMSARS specified by Palma et al. (2021);
c) to evaluate the efficacy of aslo0452 ngl-3 versus placebo, as measured by the change from baseline to Week 52 on the total UMSARS;
d) to evaluate the efficacy of aslo0452 ngl-3 versus placebo, as measured by the change from baseline to Week 52 on the Part II UMSARS;
e) to evaluate the efficacy of aslo0452 ngl-3 versus placebo, as measured by the change from baseline to Week 52 on the Clinical Global Impression-Severity (CGI-S) scale;
f) to evaluate the efficacy of aslo0452 ngl-3 versus placebo on the Scales for Outcomes in Parkinson's Disease-Autonomic Dysfunction (SCOPA-AUT); and
g) to evaluate the efficacy of aslo0452 ngl-3 versus placebo, as measured by overall survival at 52 weeks.

### Exploratory Objectives:

The exploratory molecular and imaging biomarker objectives include:
a) to evaluate the effect of aslo0452 ngl-3 versus placebo on molecular biomarkers, as measured by the change from baseline to Week 52 on levels of plasma and CSF biomarkers (including but not limited to total αSYN levels as well as CSF free αSYN levels).

### REFERENCES

Altschul, Stephen F., et al. ‘Basic Local Alignment Search Tool’. Journal of Molecular Biology, vol. 215, no. 3, October 1990, pp. 403-10. DOI.org (Crossref), https://doi.org/10.1016/S0022-2836(05)80360-2.
Angot, Elodie, and Patrik Brundin. ‘Dissecting the Potential Molecular Mechanisms Underlying α-Synuclein Cell-to-Cell Transfer in Parkinson's Disease’. Parkinsonism & Related Disorders, vol. 15, December 2009, pp. S143-47. DOI.org (Crossref), https://doi.org/10.1016/S1353-8020(09)70802-8.
Bae, E. J., et al. ‘Antibody-Aided Clearance of Extracellular Alpha-Synuclein Prevents Cell-to-Cell Aggregate Transmission’. Journal of Neuroscience, vol. 32, no. 39, September 2012, pp. 13454-69. DOI.org (Crossref), https://doi.org/10.1523/JNEUROSCI. 1292-12.2012.
Barone, Daniel A., and Claire Henchcliffe. ‘Rapid Eye Movement Sleep Behavior Disorder and the Link to Alpha-Synucleinopathies’. Clinical Neurophysiology, vol. 129, no. 8, August 2018, pp. 1551-64. DOI.org (Crossref), https://doi.org/10.1016/j.clinph.2018.05.003.
Bird, Robert E., et al. ‘Single-Chain Antigen-Binding Proteins’. Science, vol. 242, no. 4877 October 1988, pp. 423-26. DOI.org (Crossref), https://doi.org/10.1126/science.3140379.
Bisaglia, Marco, et al. ‘Structural Insights on Physiological Functions and Pathological Effects of Alpha-synuclein’. The FASEB Journal, vol. 23, no. 2, February 2009, pp. 329-40. DOI.org (Crossref), https://doi.org/10.1096/fj.08-119784.
Busner, Joan, and Steven D. Targum. ‘The Clinical Global Impressions Scale: Applying a Research Tool in Clinical Practice’. Psychiatry (Edgmont (Pa.: Township)), vol. 4, no. 7, July 2007, pp. 28-37.
Chothia, Cyrus, and Arthur M. Lesk. ‘Canonical Structures for the Hypervariable Regions of Immunoglobulins’. Journal of Molecular Biology, vol. 196, no. 4, August 1987, pp. 901-17. DOI.org (Crossref), https://doi.org/10.1016/0022-2836(87)90412-8.
Coon, Elizabeth A., et al. ‘Pure Autonomic Failure’. Mayo Clinic Proceedings, vol. 94, no. 10, October 2019, pp. 2087-98. DOI.org (Crossref), https://doi.org/10.1016/j.mayocp.2019.03.009.
Cremades, Nunilo, et al. ‘Direct Observation of the Interconversion of Normal and Toxic Forms of α-Synuclein’. Cell, vol. 149, no. 5, May 2012, pp. 1048-59. DOI.org (Crossref), https://doi.org/10.1016/j.cell.2012.03.037.
‘Current Protocols in Molecular Biology, Edited by M. Ausubel, R. Brent, R. E. Kingston, D.D. Moore, J.G. Seidman, J.A. Smith, and K. Struhl. Volumes 1 and 2. John Wiley & Sons, Inc., Media, PA, 1988, $165.00’. Molecular Reproduction and Development, vol. 1, no. 2, 1989, pp. 146-146. DOI.org (Crossref), https://doi.org/10.1002/mrd.1080010210.
Dall'Acqua, William F., et al. ‘Properties of Human IgG1s Engineered for Enhanced Binding to the Neonatal Fc Receptor (FcRn)’. Journal of Biological Chemistry, vol. 281, no. 33, August 2006, pp. 23514-24. DOI.org (Crossref), https://doi.org/10.1074/jbc.M604292200.
Danzer, K. M., et al. ‘Different Species of-Synuclein Oligomers Induce Calcium Influx and Seeding’. Journal of Neuroscience, vol. 27, no. 34, August 2007, pp. 9220-32. DOI.org (Crossref), https://doi.org/10.1523/JNEUROSCI.2617-07.2007.
Desplats, Paula, et al. ‘Inclusion Formation and Neuronal Cell Death through Neuron-to-Neuron Transmission of α-Synuclein’. Proceedings of the National Academy of Sciences, vol. 106, no. 31, August 2009, pp. 13010-15. DOI.org (Crossref), https://doi.org/10.1073/pnas.0903691106.
Emmanouilidou, Evangelia, et al. ‘Assessment of α-Synuclein Secretion in Mouse and Human Brain Parenchyma’. PLOS ONE, edited by Mark R. Cookson, vol. 6, no. 7, July 2011, p. e22225. DOI.org (Crossref), https://doi.org/10.1371/journal.pone.0022225.
Fortin, D. L. ‘Neural Activity Controls the Synaptic Accumulation of -Synuclein’. Journal of Neuroscience, vol. 25, no. 47, November 2005, pp. 10913-21. DOI.org (Crossref), https://doi.org/10.1523/JNEUROSCI.2922-05.2005.
Frauscher, Birgit, et al. ‘Validation of the Innsbruck REM Sleep Behavior Disorder Inventory: Validation of the Innsbruck RBD Inventory’. Movement Disorders, vol. 27, no. 13, November 2012, pp. 1673-78. DOI.org (Crossref), https://doi.org/10.1002/mds.25223.
Games, D., et al. ‘Reducing C-Terminal-Truncated Alpha-Synuclein by Immunotherapy Attenuates Neurodegeneration and Propagation in Parkinson's Disease-Like Models’. Journal of Neuroscience, vol. 34, no. 28, July 2014, pp. 9441-54. DOI.org (Crossref), https://doi.org/10.1523/JNEUROSCI.5314-13.2014.
Gilat, Moran, et al. ‘Melatonin for Rapid Eye Movement Sleep Behavior Disorder in Parkinson's Disease: A Randomised Controlled Trial’. Movement Disorders, vol. 35, no. 2, February 2020, pp. 344-49. DOI.org (Crossref), https://doi.org/10.1002/mds.27886.
Gilman, S., et al. ‘Second Consensus Statement on the Diagnosis of Multiple System Atrophy’. Neurology, vol. 71, no. 9, August 2008, pp. 670-76. DOI.org (Crossref), https://doi.org/10.1212/01.wnl.0000324625.00404.15.
Goetz, Christopher G., et al. ‘Movement Disorder Society-Sponsored Revision of the Unified Parkinson's Disease Rating Scale (MDS-UPDRS): Scale Presentation and Clinimetric Testing Results: MDS-UPDRS: Clinimetric Assessment’. Movement Disorders, vol. 23, no. 15, November 2008, pp. 2129-70. DOI.org (Crossref), https://doi.org/10.1002/mds.22340.
Guy, W. ECDEU assessment manual for psychopharmacology. 1976.
Hargreaves, K., et al. ‘A New and Sensitive Method for Measuring Thermal Nociception in Cutaneous Hyperalgesia’. Pain, vol. 32, no. 1, January 1988, pp. 77-88. DOI.org (Crossref), https://doi.org/10.1016/0304-3959(88)90026-7.
Herukka, Sanna-Kaisa, et al. ‘Amyloid-β and Tau Dynamics in Human Brain Interstitial Fluid in Patients with Suspected Normal Pressure Hydrocephalus’. Journal of Alzheimer's Disease, vol. 46, no. 1, May 2015, pp. 261-69. DOI.org (Crossref), https://doi.org/10.3233/JAD-142862.
Huston, J. S., et al. ‘Protein Engineering of Antibody Binding Sites: Recovery of Specific Activity in an Anti-Digoxin Single-Chain Fv Analogue Produced in Escherichia Coli.’ Proceedings of the National Academy of Sciences, vol. 85, no. 16, August 1988, pp. 5879-83. DOI.org (Crossref), https://doi.org/10.1073/pnas.85.16.5879.
Iwai, Akihiko, et al. ‘The Precursor Protein of Non-AB Component of Alzheimer's Disease Amyloid Is a Presynaptic Protein of the Central Nervous System’. Neuron, vol. 14, no. 2, February 1995, pp. 467-75. DOI.org (Crossref), https://doi.org/10.1016/0896-6273(95)90302-X.
Jellinger, Kurt A. ‘Neuropathological Spectrum of Synucleinopathies’. Movement Disorders, vol. 18, no. S6, September 2003, pp. 2-12. DOI.org (Crossref), https://doi.org/10.1002/mds. 10557.
Kabat. Sequences of Proteins of Immunological Interest. 5th ed., 1991. Kalia, Lorraine V., and Anthony E. Lang. ‘Parkinson's Disease’. The Lancet, vol. 386, no. 9996 August 2015, pp. 896-912. DOI.org (Crossref), https://doi.org/10.1016/S0140-6736(14)61393-3.
Kaufmann, Horacio, et al. ‘The Orthostatic Hypotension Questionnaire (OHQ): Validation of a Novel Symptom Assessment Scale’. Clinical Autonomic Research, vol. 22, no. 2, April 2012, pp. 79-90. DOI.org (Crossref), https://doi.org/10.1007/s10286-011-0146-2. Lashuel, Hilal A., et al. ‘Amyloid Pores from Pathogenic Mutations’. Nature, vol. 418, no. 6895 July 2002, pp. 291-291. DOI.org (Crossref), https://doi.org/10.1038/418291a.
Lawand, Nada B., et al. ‘Targeting α-Synuclein as a Therapeutic Strategy for Parkinson's Disease’. Expert Opinion on Therapeutic Targets, vol. 19, no. 10, October 2015, pp. 1351-60. DOI.org (Crossref), https://doi.org/10.1517/14728222.2015.1062877.
Lee, He-Jin, et al. ‘Direct Transfer of α-Synuclein from Neuron to Astroglia Causes Inflammatory Responses in Synucleinopathies’. Journal of Biological Chemistry, vol. 285, no. 12, March 2010, pp. 9262-72. DOI.org (Crossref), https://doi.org/10.1074/jbc.M109.081125.
Lee, Seung-Jae, et al. ‘Cell-to-Cell Transmission of Non-Prion Protein Aggregates’. Nature Reviews Neurology, vol. 6, no. 12, December 2010, pp. 702-06. DOI.org (Crossref), https://doi.org/10.1038/nrneurol.2010.145.
Luk, Kelvin C., Cheng Song, et al. ‘Exogenous α-Synuclein Fibrils Seed the Formation of Lewy Body-like Intracellular Inclusions in Cultured Cells’. Proceedings of the National Academy of Sciences, vol. 106, no. 47, November 2009, pp. 20051-56. DOI.org (Crossref), https://doi.org/10.1073/pnas.0908005106.
Luk, Kelvin C., Victoria M. Kehm, et al. ‘Intracerebral Inoculation of Pathological α-Synuclein Initiates a Rapidly Progressive Neurodegenerative α-Synucleinopathy in Mice’. Journal of Experimental Medicine, vol. 209, no. 5, May 2012, pp. 975-86. DOI.org (Crossref), https://doi.org/10.1084/jem.20112457.
Luk, Kelvin C., Victoria Kehm, et al. ‘Pathological α-Synuclein Transmission Initiates Parkinson-like Neurodegeneration in Nontransgenic Mice’. Science, vol. 338, no. 6109 November 2012, pp. 949-53. DOI.org (Crossref), https://doi.org/10.1126/science. 1227157.
Masliah, Eliezer, et al. ‘Passive Immunization Reduces Behavioral and Neuropathological Deficits in an Alpha-Synuclein Transgenic Model of Lewy Body Disease’. PLOS ONE, edited by Grainne M. McAlonan, vol. 6, no. 4, April 2011, p. e19338. DOI.org (Crossref), https://doi.org/10.1371/journal.pone.0019338.
Mollenhauer, Brit, et al. ‘α-Synuclein in Human Cerebrospinal Fluid Is Principally Derived from Neurons of the Central Nervous System’. Journal of Neural Transmission, vol. 119, no. 7, July 2012, pp. 739-46. DOI.org (Crossref), https://doi.org/10.1007/s00702-012-0784-0.
Nasreddine, Ziad S., et al. ‘The Montreal Cognitive Assessment, MoCA: A Brief Screening Tool For Mild Cognitive Impairment: MOCA: A BRIEF SCREENING TOOL FOR MCI’. Journal of the American Geriatrics Society, vol. 53, no. 4, April 2005, pp. 695-99. DOI.org (Crossref), https://doi.org/10.1111/j. 1532-5415.2005.53221.x.
Needleman, Saul B., and Christian D. Wunsch. ‘A General Method Applicable to the Search for Similarities in the Amino Acid Sequence of Two Proteins’. Journal of Molecular Biology, vol. 48, no. 3, March 1970, pp. 443-53. DOI.org (Crossref), https://doi.org/10.1016/0022-2836(70)90057-4.
Oganesyan, Vaheh, et al. ‘Structural Characterization of a Human Fc Fragment Engineered for Lack of Effector Functions’. Acta Crystallographica Section D Biological Crystallography, vol. 64, no. 6, June 2008, pp. 700-04. DOI.org (Crossref), https://doi.org/10.1107/S0907444908007877.
Oueslati, Abid, et al. ‘Role of Post-Translational Modifications in Modulating the Structure, Function and Toxicity of α-Synuclein’. Progress in Brain Research, vol. 183, Elsevier, 2010, pp. 115-45. DOI.org (Crossref), https://doi.org/10.1016/S0079-6123(10)83007-9.
Palma, Jose-Alberto, et al. ‘Limitations of the Unified Multiple System Atrophy Rating Scale as Outcome Measure for Clinical Trials and a Roadmap for Improvement’. Clinical Autonomic Research, vol. 31, no. 2, April 2021, pp. 157-64. DOI.org (Crossref), https://doi.org/10.1007/s10286-021-00782-w.
Pearson, W. R., and D. J. Lipman. ‘Improved Tools for Biological Sequence Comparison.’ Proceedings of the National Academy of Sciences, vol. 85, no. 8, April 1988, pp. 2444-48. DOI.org (Crossref), https://doi.org/10.1073/pnas.85.8.2444.
Recasens, Ariadna, and Benjamin Dehay. ‘Alpha-Synuclein Spreading in Parkinson's Disease’. Frontiers in Neuroanatomy, vol. 8, December 2014. DOI.org (Crossref), https://doi.org/10.3389/fnana.2014.00159.
Rey, Nolwen L., et al. ‘Transfer of Human α-Synuclein from the Olfactory Bulb to Interconnected Brain Regions in Mice’. Acta Neuropathologica, vol. 126, no. 4, October 2013, pp. 555-73. DOI.org (Crossref), https://doi.org/10.1007/s00401-013-1160-3.
Shen, Si-Si, et al. ‘Validation Study of REM Sleep Behavior Disorder Questionnaire -Hong Kong (RBDQ-HK) in East China’. Sleep Medicine, vol. 15, no. 8, August 2014, pp. 952-58. DOI.org (Crossref), https://doi.org/10.1016/j.sleep.2014.03.020.
Smith, Temple F., and Michael S. Waterman. ‘Comparison of Biosequences’. Advances in Applied Mathematics, vol. 2, no. 4, December 1981, pp. 482-89. DOI.org (Crossref), https://doi.org/10.1016/0196-8858(81)90046-4.
Spillantini, Maria Grazia, and Michel Goedert. ‘The α-Synucleinopathies: Parkinson's Disease, Dementia with Lewy Bodies, and Multiple System Atrophy’. Annals of the New York Academy of Sciences, vol. 920, no. 1, January 2006, pp. 16-27. DOI.org (Crossref), https://doi.org/10.1111/j. 1749-6632.2000.tb06900.x.
Tran, Hien T., et al. ‘α-Synuclein Immunotherapy Blocks Uptake and Templated Propagation of Misfolded α-Synuclein and Neurodegeneration’. Cell Reports, vol. 7, no. 6, June 2014, pp. 2054-65. DOI.org (Crossref), https://doi.org/10.1016/j.celrep.2014.05.033.
Tsigelny, Igor F., et al. ‘Dynamics of α-Synuclein Aggregation and Inhibition of Pore-like Oligomer Development by β-Synuclein: Modeling of a-Syn Oligomer Formation’. FEBS Journal, vol. 274, no. 7, April 2007, pp. 1862-77. DOI.org (Crossref), https://doi.org/10.1111/j.1742-4658.2007.05733.x.
Visser, Martine, et al. ‘Assessment of Autonomic Dysfunction in Parkinson's Disease: The SCOPA-AUT’. Movement Disorders, vol. 19, no. 11, November 2004, pp. 1306-12. DOI.org (Crossref), https://doi.org/10.1002/mds.20153.
Volpicelli-Daley, Laura A., et al. ‘Exogenous α-Synuclein Fibrils Induce Lewy Body Pathology Leading to Synaptic Dysfunction and Neuron Death’. Neuron, vol. 72, no. 1, October 2011, pp. 57-71. DOI.org (Crossref), https://doi.org/10.1016/j.neuron.2011.08.033.
Ward, E. Sally, et al. ‘Binding Activities of a Repertoire of Single Immunoglobulin Variable Domains Secreted from Escherichia Coli’. Nature, vol. 341, no. 6242 October 1989, pp. 544-46. DOI.org (Crossref), https://doi.org/10.1038/341544a0.
Wenning, Gregor K., et al. ‘Development and Validation of the Unified Multiple System Atrophy Rating Scale (UMSARS)’. Movement Disorders, vol. 19, no. 12, December 2004, pp. 1391-402. DOI.org (Crossref), https://doi.org/10.1002/mds.20255.
Wenning, Gregor K., et al. ‘The Movement Disorder Society Criteria for the Diagnosis of Multiple System Atrophy’. Movement Disorders, vol. 37, no. 6, June 2022, pp. 1131-48. DOI.org (Crossref), https://doi.org/10.1002/mds.29005
Winner, Beate, et al. ‘In Vivo Demonstration That α-Synuclein Oligomers Are Toxic’. Proceedings of the National Academy of Sciences, vol. 108, no. 10, March 2011, pp. 4194-99. DOI.org (Crossref), https://doi.org/10.1073/pnas.1100976108.

1. A method for treating or preventing an α-synucleinopathy in a subject in need thereof, the method comprising administering to the subject a fixed dose of 50-5,000 mg of an anti-α-synuclein antibody, or antigen-binding fragment thereof.

2. The method according to claim 1, wherein the fixed dose of the anti-α-synuclein antibody, or antigen-binding fragment thereof, is:
(a) 70-4,500 mg;
(b) 1,000-4,500 mg;
(c) 1,800-2,600 mg; or
(d) 2.000-2,400 mg.

3-6. (canceled)

7. The method according to claim 1, wherein the fixed dose of the anti-α-synuclein antibody, or antigen-binding fragment thereof, is:
(a) 2,000 mg;
(b) 2,400 mg;
(c) 1,400 mg:
(d) 1,600 mg;
(e) 1,700 mg
(f) 1,800 mg; or
(g) 4,500 mg.

8-9. (canceled)

10. The method according claim 1, wherein the method comprises administering the anti-α-synuclein antibody, or antigen-binding fragment thereof, intravenously, subcutaneously, intradermally, or intramuscularly.

11. (canceled)

12. The method according to any one of the preceding claimsclaim 1, wherein the anti-α-synuclein antibody, or antigen-binding fragment thereof, is administered to the subject:
(a) once every 3-5 weeks;
(b) multiple times over a period of at least 3 months, 6 months, 9 months, 1 year, 2 years or 5 years; or
(c) once every four weeks over a period of at least 3 months, at least 6 months, at least 9 months, at least 1 year, at least 2 years or at least 5 years.

13-14. (canceled)

15. The method according to claim 1, wherein the α-synucleinopathy is selected from Parkinson's disease (PD), dementia with Lewy bodies (DLB), multiple system atrophy (MSA), Alzheimer's Disease, pure autonomic failure, REM behavior disorder, a prodromal synucleinopathy and a neuroaxonal dystrophy.

16-18. (canceled)

19. The method according to claim 15, wherein the subject has a diagnosis of possible or probable MSA.

20. The method according claim 1, wherein:
(a) the subject to be treated has: a Unified Multiple System Atrophy Rating Scale (UMSARS) Part I score of 21 or less;
(b) the subject to be treated has:
a) a severity score of 2 or less on the swallowing item;
b) a severity score of 2 or less on the ambulation item; or
c) a severity score of 2 or less on the falling item
as measured using a Unified Multiple System Atrophy Rating Scale (UMSARS) Part I score; or
(c) the subject to be treated has a Unified Multiple System Atrophy Rating Scale (UMSARS) Part IV disability score of 3 or less.

21-40. (canceled)

41. The method according to claim 1, wherein the anti-α-synuclein antibody, or antigen-binding fragment thereof, comprises:
a) three heavy chain CDRs having sequences:
(i) H-CDR1 of SEQ ID NO: 5;
(ii) H-CDR2 of SEQ ID NO: 15; and
(iii) H-CDR3 of SEQ ID NO; 16; and
b) three light chain CDRs having sequences:
(i) L-CDR1 of SEQ ID NO: 20;
(ii) L-CDR2 of SEQ ID NO: 10; and
(iii) L-CDR3 of SEQ ID NO: 21.

42. The method according to claim 41, wherein the anti-α-synuclein antibody or antigen-binding fragment thereof, comprises a variable heavy chain region comprising an amino acid sequence that is at least 90% identical to the amino acid sequence of SEQ ID NO: 14.

43. The method according to claim 41, wherein the anti-α-synuclein antibody or antigen-binding fragment thereof, comprises a variable heavy chain region comprising the amino acid sequence of SEQ ID NO: 14.

44. The method according to claim 41, wherein the anti-α-synuclein antibody or antigen-binding fragment thereof, comprises a variable light chain region comprising an amino acid sequence that is at least 90% identical to the amino acid sequence of SEQ ID NO: 19.

45. The method according to claim 41, wherein the anti-α-synuclein antibody or antigen-binding fragment thereof, comprises a variable light chain region comprising the amino acid sequence of SEQ ID NO: 19.

46. The method according to claim 41, wherein the anti-α-synuclein antibody or antigen-binding fragment thereof, comprises a variable heavy chain region comprising an amino acid sequence that is at least 95% identical to the amino acid sequence of SEQ ID NO: 14 and a variable light chain region comprising an amino acid sequence that is at least 95% identical to the amino acid sequence of SEQ ID NO: 19.

47. The method according to claim 46, wherein the anti-α-synuclein antibody or antigen-binding fragment thereof, comprises a variable heavy chain region comprising the amino acid sequence of SEQ ID NO: 14 and a variable light chain region comprising the amino acid sequence of SEQ ID NO: 19.

48. The method according to claim 41, wherein the anti-α-synuclein antibody or antigen-binding fragment thereof, comprises a heavy chain comprising an amino acid sequence that is at least 90% identical to the amino acid sequence of SEQ ID NO: 12 or SEQ ID NO: 62.

49. The method according to claim 48, wherein the anti-α-synuclein antibody or antigen-binding fragment thereof, comprises a heavy chain comprising the amino acid sequence of SEQ ID NO: 12 or SEQ ID NO: 62.

50. The method according to claim 41, wherein the anti-α-synuclein antibody or antigen-binding fragment thereof, comprises a light chain region comprising an amino acid sequence that is at least 90% identical to the amino acid sequence of SEQ ID NO: 17.

51. The method according to claim 50, wherein the anti-α-synuclein antibody or antigen-binding fragment thereof, comprises a light chain region comprising the amino acid sequence of SEQ ID NO: 17.

52. The method according to claim 41, wherein the anti-α-synuclein antibody or antigen-binding fragment thereof, comprises a heavy chain comprising an amino acid sequence that is at least 95% identical to the amino acid sequence of SEQ ID NO: 12 or SEQ ID NO: 62 and a light chain comprising an amino acid sequence that is at least 95% identical to the amino acid sequence of SEQ ID NO: 17.

53. The method according to claim 52, wherein the anti-α-synuclein antibody or antigen-binding fragment thereof, comprises a heavy chain comprising the amino acid sequence of SEQ ID NO: 12 or SEQ ID NO: 62 and a light chain comprising the amino acid sequence of SEQ ID NO: 17.

54. The method according to claim 1, wherein the anti-α-synuclein antibody or antigen-binding fragment thereof, is an antibody.

55. The method according to claim 1, wherein the anti-α-synuclein antibody or antigen-binding thereof, comprises a triple mutation in the Fc region corresponding to L234F/L235E/P331S numbered based on the Kabat numbering.

56. (canceled)

57. A kit comprising:
a) an anti-α-synuclein antibody, or antigen-binding fragment thereof; and
b) instructions for use of the same for treating an α-synucleinopathy at a fixed dose of 50-5,000 mg.


SEQUENCE LISTING (62 sequences)

SEQ ID NO: 1 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 122 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAGGAVNVYYYYGMDVWGQGTMVTVSS

SEQ ID NO: 3 - protein, 115 aa, synthetic construct
QAVLTQPSSLSASPGASASLTCTLRSGNNVGNYRIYWYQQKSGSPPQYLLRYKSDADKHQGSGVPSRFSGSKDASANAGILFISGLQSEDEADYYCMVWHSGAWVFGGGTKLTVL

SEQ ID NO: 4 - protein, 122 aa, synthetic construct
  VARIANT 50: note = X50 = S or A
  VARIANT 53: note = X53 = H or G
  VARIANT 54: note = X54 = L or S
  VARIANT 101: note = X101 = N or R
  VARIANT 102: note = X102 = H or R
  VARIANT 103: note = X103 = V or G
  VARIANT 104: note = X104 = K or R
  VARIANT 105: note = X105 = Y or I
EVQLLESGGGLVQPGGSLRLSCAASGFTFSSYAMSWVRQAPGKGLEWVSXISXXGGSTYYADSVKGRFTISRDNSKNTLYLQMNSLRAEDTAVYYCAGGAXXXXXYYGMDVWGQGTMVTVSS

SEQ ID NO: 5 - protein, 5 aa, synthetic construct
SYAMS

SEQ ID NO: 6 - protein, 16 aa, synthetic construct
  VARIANT 1: note = X1 = S or A
  VARIANT 4: note = X4 = H or G
  VARIANT 5: note = X5 = L or S
XISXXGGSTYYADSVK

SEQ ID NO: 7 - protein, 13 aa, synthetic construct
  VARIANT 3: note = X3 = N or R
  VARIANT 4: note = X4 = H or R
  VARIANT 5: note = X5 = V or G
  VARIANT 6: note = X6 = K or R
  VARIANT 7: note = X7 = Y or I
GAXXXXXYYGMDV

SEQ ID NO: 8 - protein, 114 aa, synthetic construct
  VARIANT 27: note = X27 = G or S
  VARIANT 28: note = X28 = A or G
  VARIANT 29: note = X29 = P or D
  VARIANT 30: note = X30 = L or F
  VARIANT 31: note = X31 = P or S
  VARIANT 32: note = X32 = K or R
  VARIANT 42: note = X42 = S or P
  VARIANT 82: note = X82 = F or L
  VARIANT 99: note = X99 = D or S
  VARIANT 100: note = X100 = H or S
  VARIANT 102: note = X102 = V or A
  VARIANT 104: note = X104 = V or Y
QAVLTQPSSLSASPGASASLTCTLRSXXXXXXYRIYWYQQKXGSPPQYLLRYKSDADKHQGSGVPSRFSGSKDASANAGILXSGLQSEDEADYYCMVWXXGXWXFGGGTKLTVL

SEQ ID NO: 9 - protein, 14 aa, synthetic construct
  VARIANT 5: note = X5 = G or S
  VARIANT 6: note = X6 = A or G
  VARIANT 7: note = X7 = P or D
  VARIANT 9: note = X9 = V or Y
TLRSXXXXXXYRIY

SEQ ID NO: 10 - protein, 11 aa, synthetic construct
YKSDADKHQGS

SEQ ID NO: 11 - protein, 9 aa, synthetic construct
  VARIANT 4: note = X4 = D or S
  VARIANT 5: note = X5 = H or S
  VARIANT 7: note = X7 = V or A
  VARIANT 9: note = X9 = V or Y
MVWXXGXWX

SEQ ID NO: 12 - protein, 452 aa, synthetic construct
EVQLLESGGGLVQPGGSLRLSCAASGFTFSSYAMSWVRQAPGKGLEWVSSISHLGGSTYYADSVKGRFTISRDNSKNTLYLQMNSLRAEDTAVYYCAGGANHGKYYYGMDKWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 366 bp, synthetic construct
  source 1..366: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcatccatttcccaccttggtggtagcacatactacgcagactccgtgaagggccggttcaccatctccagagacaattccaagaacacgctgtatctgcaaatgaacagcctgagagccgaggacacggccgtgtattactgtgcgggaggggcaaaccacgggaagtactactacggaatggacaagtggggccaagggaccacggtcaccgtctcctca

SEQ ID NO: 14 - protein, 121 aa, synthetic construct
EVQLLESGGGLVQPGGSLRLSCAASGFTFSSYAMSWVRQAPGKGLEWVSSISHLGGSTYYADSVKGRTISRDNSKNTLYLQMNSLRAEDTAVYYCAGGANHGKYYYGMDKWGQGTTVTVSS

SEQ ID NO: 15 - protein, 17 aa, synthetic construct
SISHLGGSTYYADSVKG

SEQ ID NO: 16 - protein, 13 aa, synthetic construct
GANHGKYYYGMDK

SEQ ID NO: 17 - protein, 221 aa, synthetic construct
QAVLTQPASLSASPGASASLTCTLRSGAPLPKYRIYWYQQKPGSPPQYLLRYKSDADKHQGSGVPSRFSGSKDASANAGILLISGLQSEDEADYYCMVWDHGVWYFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 18 - protein, 345 aa, synthetic construct
CAGGCTGTGCTGACTCAGCCGGCTTCCCTCTCTGCGTCTCCTGGAGCATCAGCCAGTCTCACCTGCACCTTGCGCAGTGGGGCGCCCCTGCCGAAGTATAGGATATACTGGTATCAGCAGAAGCCAGGGAGTCCTCCCCAGTATCTCCTGAGGTACAAATCAGACGCAGATAAACACCAGGGCTCTGGAGTCCCCAGCCGCTTTTCTGGATCCAAAGATGCTTCGGCCAATGCAGGGATTTTACTCATCTCTGGGCTCCAGTCTGAGGATGAGGCTGACTATTATTGTATGGTTTGGGACCACGGCGTCTGGTATTTCGGCGGAGGGACCAAGCTGACCGTCCTA

SEQ ID NO: 19 - protein, 115 aa, synthetic construct
QAVLTQPASLSASPGASASLTCTLRSGAPLPKYRIYWYQQKPGSPPQYLLRYKSDADKHQGSGVPSRFSGSKDASANAGILLISGLQSEDEADYYCMVWDHGVWYFGGGTKLTVL

SEQ ID NO: 20 - protein, 14 aa, synthetic construct
TLRSGAPLPKYRIY

SEQ ID NO: 21 - protein, 9 aa, synthetic construct
MVWDHGVWY

SEQ ID NO: 22 - protein, 452 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAGGARRGRIYYGMDKWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 366 bp, synthetic construct
  source 1..366: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgggaggggcacggcgcgggcgcatctactacggaatggacaaatggggccaagggacaacggtcaccgtctcctca

SEQ ID NO: 24 - protein, 122 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAGGARRGRIYYGMDKWGQGTTVTVSS

SEQ ID NO: 25 - protein, 5 aa, synthetic construct
SYAMS

SEQ ID NO: 26 - protein, 17 aa, synthetic construct
AISGSGGSTYYADSVKG

SEQ ID NO: 27 - protein, 13 aa, synthetic construct
GARRGRIYYGMDK

SEQ ID NO: 28 - protein, 221 aa, synthetic construct
QAVLTQPASLSASPGASASLTCTLRSSGDFSRYRIYWYQQKPGSPPQYLLRYKSDADKHQGSGVPSRFSGSKDASANAGILLISGLQSEDEADYYCMVWSSGAWYFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 29 - DNA, 345 bp, synthetic construct
  source 1..345: mol_type = other DNA
caggctgtgctgactcagccggcttccctctctgcgtctcctggagcatcagccagtctcacctgcaccttgcgcagttccggggacttctcccggtataggatatactggtatcagcagaagccagggagtcctccccagtatctcctgaggtacaaatcagacgcagataaacaccagggctctggagtccccagccgcttttctggatccaaagatgcttcggccaatgcagggattttactcatctctgggctccagtctgaggatgaggctgactattattgtatggtttggtccagcggcgcttggtacttcggcggagggaccaagctgaccgtccta

SEQ ID NO: 30 - protein, 115 aa, synthetic construct
QAVLTQPASLSASPGASASLTCTLRSSGDFSRYRIYWYQQKPGSPPQYLLRYKSDADKHQGSGVPSRFSGSKDASANAGILLISGLQSEDEADYYCMVWSSGAWYFGGGTKLTVL

SEQ ID NO: 31 - protein, 14 aa, synthetic construct
TLRSSGDFSRYRIY

SEQ ID NO: 32 - protein, 11 aa, synthetic construct
YKSDADKHQGS

SEQ ID NO: 33 - protein, 9 aa, synthetic construct
MVWSSGAWY

SEQ ID NO: 34 - protein, 123 aa, synthetic construct
  VARIANT 1: note = X1 = Q or V
  VARIANT 11: note = X11 = V or L
  VARIANT 75: note = X75 = S or R
XVQLVQSGAEXKKPGSSVKVSCKASGGTFSSYAISWVRQAPGQGLEWMGRIIPILGTSNYAQKFQGRVTITADEXTSTAYMELSSLRSEDTAVYYCARRSSLAAADRGAFDIWGQGTMVTVSS

SEQ ID NO: 35 - protein, 108 aa, synthetic construct
  VARIANT 15: note = X15 = R, G or A
  VARIANT 28: note = X28 = T or R
  VARIANT 29: note = X29 = S, R or T
  VARIANT 32: note = X32 = A or T
  VARIANT 33: note = X33 = N or S
  VARIANT 37: note = X37 = H or Q
  VARIANT 44: note = X44 = V or I
  VARIANT 47: note = X47 = M or I
  VARIANT 50: note = X50 = K or E
  VARIANT 51: note = X51 = N or D
  VARIANT 57: note = X57 = V or I
  VARIANT 65: note = X65 = S or R
  VARIANT 68: note = X68 = N or T
  VARIANT 92: note = X92 = S or N
  VARIANT 93: note = X93 = S or T
  VARIANT 95: note = X95 = N, K or H
  VARIANT 96: note = X96 = H or P
  VARIANT 97: note = X97 = V or W
SSELTQDPAVSVALXQTVRITCQGDSLXXYYXXWYQXKPGQAPXLVXYGXXNRPSGXPDRFSGSXSGXTASLTITGAQAEDEADYYCNSRDXXGXXXVFGGGTKLTVL

SEQ ID NO: 36 - protein, 11 aa, synthetic construct
  VARIANT 6: note = X6 = T or R
  VARIANT 7: note = X7 = S, R or T
  VARIANT 10: note = X10 = A or T
  VARIANT 11: note = X11 = N or S
QGDSLXXYYXX

SEQ ID NO: 37 - protein, 7 aa, synthetic construct
  VARIANT 2: note = X2 = K or E
  VARIANT 3: note = X3 = N or D
GXXNRPS

SEQ ID NO: 38 - protein, 11 aa, synthetic construct
  VARIANT 5: note = X5 = S or N
  VARIANT 6: note = X6 = S or T
  VARIANT 8: note = X8 = N, K or H
  VARIANT 9: note = X9 = H or P
  VARIANT 10: note = X10 = V or W
NSRDXXGXXXV

SEQ ID NO: 39 - protein, 123 aa, synthetic construct
QVQLVQSGAEVKKPGSSVKVSCKASGGTFSSYAISWVRQAPGQGLEWMGRIIPILGTSNYAQKFQGRVTITADESTSTAYMELSSLRSEDTAVYYCARRSSLAAADRGAFDIWGQGTMVTVSS

SEQ ID NO: 40 - protein, 5 aa, synthetic construct
SYAIS

SEQ ID NO: 41 - protein, 17 aa, synthetic construct
RIIPILGTSNYAQKFQG

SEQ ID NO: 42 - protein, 14 aa, synthetic construct
RSSLAAADRGAFDI

SEQ ID NO: 43 - protein, 108 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 44 - protein, 11 aa, synthetic construct
QGDSLRSYYAS

SEQ ID NO: 45 - protein, 7 aa, synthetic construct
GKNNRPS

SEQ ID NO: 46 - protein, 11 aa, synthetic construct
NSRDSSGNHVV

SEQ ID NO: 47 - protein, 123 aa, synthetic construct
GVQLVQSGAELKKPGSSVKVSCKASGGTFSSYAISWVRQAPGQGLEWMGRIIPILGTSNYAQKFQGRVTITADERTSTAYMELSSLRSEDTAVYYCARRSSLAAADRGAFDIWGQGTMVTVSS

SEQ ID NO: 48 - protein, 122 aa, synthetic construct
QVQLVQSGAEVKKPGSSVKVSCKASGGTFGTYSITWVRQAPGQGLEWMGDIVPIFGTPNYAQNFQGRVTISADVSTSTVYMELSSLRSEDTAVYYCAKRGSYYGRGGWFDPWGRGTLVTVSS

SEQ ID NO: 49 - protein, 5 aa, synthetic construct
TYSIT

SEQ ID NO: 50 - protein, 17 aa, synthetic construct
DIVPIFGTPNYAQNFQG

SEQ ID NO: 51 - protein, 13 aa, synthetic construct
RGSYYGRGGWFDP

SEQ ID NO: 52 - protein, 122 aa, synthetic construct; identical to SEQ ID NO: 48

SEQ ID NO: 53 - protein, 5 aa, synthetic construct
TYSIT

SEQ ID NO: 54 - protein, 17 aa, synthetic construct
DIVPIFGTPNYAQNFQG

SEQ ID NO: 55 - protein, 13 aa, synthetic construct
RGSYYGRGGWFDP

SEQ ID NO: 56 - protein, 50 aa, synthetic construct
  REPEAT 1..50: note = This sequence may encompass 1-10 'Gly Gly Gly Gly Ser' repeating units
GGGGSGGGGSGGGGSGGGGSGGGGSGGGGSGGGGSGGGGSGGGGSGGGGS

SEQ ID NO: 57 - protein, 15 aa, synthetic construct
GGGGSGGGGSGGGGS

SEQ ID NO: 58 - protein, 98 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 59 - protein, 11 aa, synthetic construct
WGQGTTVTVSS

SEQ ID NO: 60 - protein, 102 aa, synthetic construct
QAVLTQPASLSASPGASASLTCTLRSGINVGTYRIYWYQQKPGSPPQYLLRYKSDSDKQQGSGVPSRFSGSKDASANAGILLISGLQSEDEADYYCMIWHWV

SEQ ID NO: 61 - protein, 10 aa, synthetic construct
FGGGTKLTVL

SEQ ID NO: 62 - protein, 451 aa, synthetic construct
EVQLLESGGGLVQPGGSLRLSCAASGFTFSSYAMSWVRQAPGKGLEWVSSISHLGGSTYYADSVKGRFTISRDNSKNTLYLQMNSLRAEDTAVYYCAGGANHGKYYYGMDKWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG